(19) United States
(12) Patent Application Publication
(10) Pub. No.: US 2017/0165230 A1
(43) Pub. Date: Jun. 15, 2017

(54) USE OF GSK-3 INHIBITORS OR ACTIVATORS WHICH MODULATE PD-1 OR T-BET EXPRESSION TO MODULATE T CELL IMMUNITY

(71) Applicant: Christopher RUDD, Montreal (CA)

(72) Inventors: Christopher RUDD, Montreal (CA); Dae Choon LEE, Springfield, MO (US); David Mark ROTHSTEIN, Pittsburgh, PA (US); Young Mee LEE, Springfield, MO (US)

(21) Appl. No.: 15/302,589

(22) PCT Filed: Apr. 9, 2015
(86) PCT No.: PCT/IB2015/052606

§ 371 Date: Oct. 7, 2016

Related U.S. Application Data
(60) Provisional application No. 61/977,340, filed on Apr. 9, 2014.

(51) Int. Cl. A61K 31/404; A61K 31/506; A61K 38/10; A61K 31/426; A61K 31/433; A61K 9/00

(52) U.S. Cl. CPC A61K 31/404; A61K 31/433; A61K 9/0053; A61K 38/10; A61K 31/426; A61K 31/506

(57) ABSTRACT

The present application generally relates to the discovery that glycogen synthase kinase 3 (GSK-3) is an upstream signalling molecule that controls PD-1 transcription and Tbet expression by immune cells and in particular T-cells. Based on this discovery, and in view of the known immunosuppressive effect of PD-1 on immunity and the promoting effect of Tbet on T cell immunity, the present invention relates to the use of GSK-3 inhibitors to promote immunity, including cytotoxic T cell immunity in subjects in need thereof, especially subjects with chronic conditions wherein inhibiting PD-1 expression and/or blockade or Tbet up-regulation is therapeutically desirable such as cancer and infectious conditions. Further, based on this discovery the present invention relates to the use of compounds which promote GSK-3 expression or activity to suppress immunity, especially aberrant T cell immunity in subjects in need thereof, e.g., subjects with chronic conditions wherein PD-1 upregulation or Tbet down regulation is therapeutically desirable such as allergic, autoimmune or inflammatory conditions. Also, screening methods for identifying immune agonists and antagonists, especially antibodies, are provided.

### RELATED APPLICATIONS

The present application claims benefit of priority to U.S. provisional application No. 61/977,340 filed on Apr. 9, 2014, the contents of which are incorporated by reference herein.

### FIELD

The present application generally relates to the discovery that glycogen synthase kinase 3 (GSK-3) is an upstream signalling molecule that controls PD-1 transcription and Tbet expression by immune cells and in particular expression thereof by T-cells. Based on this discovery, and in view of the known immunosuppressive effect of PD-1 on immunity and the promoting effect of Tbet on T cell immunity, the present invention relates to the use of GSK-3 inhibitors to promote immunity, including cytotoxic T cell immunity in subjects in need thereof, especially subjects with chronic conditions wherein inhibition of PD-1 transcription or expression or Tbet upregulation is therapeutically desirable such as cancer and infectious conditions. Further, based on this discovery the present invention relates to the use of GSK-3 activators to suppress immunity, especially aberrant T cell immunity in subjects in need thereof, e.g., subjects with chronic conditions wherein T cell activity is elevated such as allergic, autoimmune or inflammatory conditions.

### BACKGROUND

Immune negative checkpoint regulator (NCR) pathways have proven to be extraordinary clinical targets in the treatment of human immune-related diseases. Blockade of two NCRs, CTLA-4 and PD-1, using monoclonal antibodies (mabs) to enhance tumor immunity is revolutionizing the treatment of cancer and has established these pathways as clinically validated targets in human disease. Recently, soluble versions of NCR ligands that trigger or block NCR pathways have entered the clinic as immunosuppressive drugs to treat autoimmunity (e.g. AMP-110/B7-H4-Ig for Rheumatoid arthritis).

The present invention relates to a specific protein kinase Glycogen Synthase Kinase-3 (GSK-3) and the discovery of its role in regulation of T cell immunity. Specifically, this invention provides a greater understanding of the signaling pathways affected by this molecule and how this discovery may be exploited to regulate T cell immunity as a means of treating chronic disease conditions.

GSK-3 is a proline-directed, serine/threonine kinase for which two isoforms, GSK-3α and GSK-3β, have been identified, phosphorylates the rate-limiting enzyme of glycogen synthesis, glycogen synthase (GS). See, for example, Embi, et al., Eur. J. Biochem., 107, 519-527 (1980). GSK-3 α and GSK-3β are both highly expressed in the body. See, for example, Woodgett, et al., EMBO, 9, 2431-2438 (1990) and Loy, et al., J. Peptide Res., 54, 85-91 (1999). Besides GS, a number of other GSK-3 substrates have been identified, including many metabolic, signaling, and structural proteins. Notable among the plurality of signaling proteins regulated by GSK-3 are many transcription factors, including activator protein-1; cyclic AMP response element binding protein (CREB); the nuclear factor (NF) of activated T-cells; heat shock factor-1; β-catenin; c-Jun; c-Myc; c-Myb; and NF-KB See, for example, C. A. Grimes, et al., Prog. Neurobiol., 65, 391-426 (2001), H. Eldar-Finkelman, Trends in Molecular Medicine, 8, 126-132 (2002), and P. Cohen, et al., Nature, 2, 1-8, (2001). Accordingly, targeting the activity of GSK-3 has significant therapeutic potential in the treatment of many disparate pathologies and conditions, for example, Alzheimer's disease (A. Castro, et al., Exp. Opin. Ther. Pat., 10, 1519-1527 (2000)); asthma (P. J. Barnes, Ann. Rev. Pharmacol. Toxicol., 42, 81-98 (2002)); cancer (Beals, et al., Science, 275, 1930-1933 (1997), L. Kim, et al., Curr. Opin. Genet. Dev., 10, 508-514 (2000), and Q. Eastman, et al., Curr. Opin. Cell Biol., 11, 233 (1999)); diabetes and its related sequelae, for example, Syndrome X and obesity (S. E. Nikoulina, et al., Diabetes, 51, 2190-2198 (2002), Orena, et al., JBC, 15765-15772 (2000), and Summers, et al., J. Biol. Chem., 274 17934-17940 (1999)); hair loss (S. E. Millar, et al., Dev. Biol., 207, 133-149 (1999) and E. Fuchs, et al., Dev. Cell, 1, 13-25 (2001)); inflammation (P. Cohen, Eur. J. Biochem., 268, 5001-5010 (2001)); mood disorders, such as depression (A. Adnan, et al., Chem. Rev., 101, 2527-2540 (2001) and R. S. B. Williams, et al., Trends Phamacol. Sci., 21, 61-64 (2000)); neuronal cell death and stroke (D. A. E. Cross, et al., J. Neurochem., 77, 94-102 (2001) and C. Sasaki, et al., Neurol. Res., 23, 588-592 (2001)); bipolar disorder (Klein, et al., PNAS, 93, 8455-8459 (1996)); skeletal muscle atrophy (G. J. Brunn, et al., Science, 277, 99-101 (1997), R. E. Rhoads, J. Biol. Chem., 274, 30337-30340 (1999), V. R. Dharmesh, et al., Am. J. Physiol. Cell Physiol. 283, C545-551 (2002), and K. Baar, et al., A. J. Physiol., 276, C120-C127 (1999)); decreased sperm motility (Vijayaraghavan, et al., Biol. Reproduction, 54, 709-718 (1996)); protozoan infection, (Fugel et al., J Med. Chem., 56(1):264-75 (2013), Ojo et al., Antimicrob. Agents, Chemotherapy, 52(10):3710-7 (2008), Nurul et al., Trop Biomed., 27(3):624-31 (2010); tick infection, Fabres et al, Parasitology, 137(1):1537-46 (2010); viral replication, (Sun et al., PLos One., 7(4):e34761 (2012), Kehn-Hall et al., Virology, 415(1):56-68 (2011), Fujimuro et al., J Virol., 79:16:10429-41(2005); Wu et al., J Biol. Chem, 284(8):5229-39 (2009)) infections (cardio-protection (C. Badorff, et al., J. Clin. Invest., 109, 373-381 (2002), S. Haq, et al., J. Cell Biol., 151, 117-129 (2000), H. Tong, et al., Circulation Res., 90, 377-379 (2002), protozoan diseases (septic shock, (Martin, US Patent Publication No. 20120309807).

The invention further relates to novel therapies involving the regulation of PD-1 and/or Tbet expression, molecules respectively known to elicit a suppressive or potentiating effect on T-cell immunity. Programmed Death 1 (PD-1), also known as CD279; gene name PDCD1; accession number NP-005009 is a cell surface receptor with a critical role in regulating the balance between stimulatory and inhibitory signals in the immune system and maintaining peripheral tolerance (Ishida, Y et al. 1992 EMBO J 11 3887; Kier, Mary E et al. 2008 Annu Rev Immunol 26 677-704; Okazaki, Taku et al. 2007 International Immunology 19 813-824). It is an inhibitory member of the immunoglobulin super-family with homology to CD28. The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Expression of PD-1 is inducible on T cells, B cells, natural killer (NK) cells and monocytes, for example upon lymphocyte activation via T cell receptor (TCR) or B cell receptor (BCR) signaling (Kier, Mary E et al. 2008 Annu Rev Immunol 26 677-704; Agata, Y et al 1996 Int Immunol 8 765-72). PD-1 has two known ligands, PD-L1 (B7-H1, CD274) and PD-L2 (B7-DC, CD273), which are cell surface expressed members of the B7 family (Freeman, Gordon et al. 2000 J Exp Med 192 1027; Latchman, Y et al. 2001 Nat Immunol 2 261). Upon ligand engagement, PD-1 recruits phosphatases such as SHP-1 and SHP-2 to its intracellular tyrosine motifs which subsequently dephosphorylate effector molecules activated by TCR or BCR signaling (Chemnitz, J et al. 2004 J Immunol 173 945-954; Riley, James L 2009 Immunological Reviews 229 114-125). In this way, PD-1 transduces inhibitory signals into T and B cells when it is engaged simultaneously with the TCR or BCR. It may also affect signaling via other receptor systems.

PD-1 is a member of the immunoglobulin family of molecules (Ishida et al. (1992) EMBO J. 11:3887; Shinohara et al. (1994) Genomics 23:704). PD-1 was previously identified using a subtraction cloning based approach designed to identify modulators of programmed cell death. (Ishida et al. (1992) EMBO J. 11:3887-95; Woronicz et al. (1995) Curr. Top. Microbiol. Immunol. 200:137). PD-1 is believed to play a role in lymphocyte survival, e.g., during clonal selection (Honjo (1992) Science 258:591; Agata et al. (1996) Int. Immunology. 8:765; Nishimura et al. (1996) Int. Immunology 8:773). PD-1 was also implicated as a regulator of B cell responses (Nishimura (1998) Int. Immunology 10:1563). Unlike CTLA4, which is found only on T cells, PD-1 is also found on B cells and myeloid cells.

PD-1 has been demonstrated to down-regulate effector T cell responses via both cell-intrinsic and cell-extrinsic functional mechanisms. Inhibitory signaling through PD-1 induces a state of anergy or unresponsiveness in T cells, resulting in the cells being unable to clonally expand or produce optimal levels of effector cytokines. PD-1 may also induce apoptosis in T cells via its ability to inhibit survival signals from co-stimulation, which leads to reduced expression of key anti-apoptotic molecules such as Bcl-_{XL} (Kier, Mary E et al. 2008 Annu Rev Immunol 26 677-704). In addition to these direct effects, recent publications have implicated PD-1 as being involved in the suppression of effector cells by promoting the induction and maintenance of regulatory T cells (T_{REG}) and other suppressor T-cell subsets (i.e. generate IL-10). For example, PD-L1 expressed on dendritic cells was shown to act in synergy with TGFβ to promote the induction of CD4⁺ FoxP3+ T_{REG} with enhanced suppressor function (Francisco, Loise M et al. 2009 J Exp Med 206 3015-3029).

The first indication of the importance of PD-1 in peripheral tolerance and inflammatory disease came from the observation that PD-1 knockout (Pdcd1^{-/-}) mice develop spontaneous autoimmunity. Fifty percent of Pdcd1^{-/-} mice on a C57BL/6 background develop lupus-like glomerulonephritis and arthritis by 14 months of age and BALB/c-Pdcd1^{-/-} mice develop a fatal dilated cardiomyopathy and production of autoantibodies against cardiac troponin I from 5 weeks onwards (Nishimura, H et al. 1999 Immunity 11 141-151; Nishimura, H et al. 2001 Science 291 319-322). Furthermore, introduction of PD-1 deficiency to the non-obese diabetic (NOD) mouse strain dramatically accelerates the onset and incidence of diabetes resulting in all NOD-Pdcd1^{-/-} mice developing diabetes by 10 weeks of age (Wang, J et al. 2005 Proc. Natl. Acad. Sci. USA 102 11823). Additionally, using induced murine models of autoimmunity such as experimental autoimmune encephalomyelitis (EAE), or transplantation/graft-versus-host (GVHD) models, several groups have shown that blocking the PD-1-PD-L interaction exacerbates disease, further confirming the key role of PD-1 in inflammatory diseases. Importantly, evidence suggests that PD-1 has a comparable immune modulatory function in humans as mice, as polymorphisms in human PDCD1 have been associated with a range of autoimmune diseases including systemic lupus erythematosus (SLE), multiple sclerosis (MS), type I diabetes (TID), rheumatoid arthritis (RA) and Grave's disease (Okazaki, Taku et al. 2007 International Immunology 19 813-824; Prokunina, L et al. 2002 Nat Genet 32 666-669; Kroner, A et al. 2005 Ann Neurol 58 50-57; Prokunina, L et al 2004 Arthritis Rheum 50 1770).

Several therapeutic approaches to enhance PD-1 signaling and modulate inflammatory disease have been reported, using murine models of autoimmunity. One such approach tried was to generate artificial dendritic cells which over-express PD-L1. Injection of mice with antigen-loaded PD-L1-dendritic cells before or after induction of EAE by MOG peptide immunization reduced the inflammation of the spinal cord as well as the clinical severity of the disease (Hirata, S et al. 2005 J Immunol 174 1888-1897). Another approach was to try to cure lupus-like syndrome in BXSB mice by delivering a PD-1 signal using a recombinant adenovirus expressing mouse PD-L1. Injection of this virus partially prevented the development of nephritis as shown by lower frequency of proteinuria, reduced serum anti-dsDNA Ig and better renal pathology (Ding, H et al. 2006 Clin Immunol 118 258). These results suggest that enhancing the PD-1 signal could have therapeutic benefit in human autoimmune disease. An alternative therapeutic approach more appropriate as a human drug treatment would be to use an agonistic monoclonal antibody against human PD-1. Binding of this agonistic antibody would ideally independently transduce inhibitory signals through PD-1 whilst also synergizing with ongoing endogenous signals emanating from the natural PD-1-PD1-L interaction. An agonistic anti-PD-1 mAb would be predicted to modulate a range of immune cell types involved in inflammatory disease including T cells, B cells, NK cells and monocytes and would therefore have utility in the treatment of a wide range of human autoimmune or inflammatory disorders.

PD-1 also plays a central role in the development of T-cell exhaustion of CD4⁺ and CD8⁺ T cells (Barber et al., 2006 Nature 439, 682-68; Day et al., 2006 Nature 443, 350-354; Freeman et al., 2006 J Exp Med 203, 2223-2227.). This exhaustion state develops during many chronic infections and cancer and results T-cell dysfunction with poor effector responses and a sustained expression of inhibitory receptors such as PD-1. Exhaustion prevents optimal control of infection and tumors. PD-1 expression was first observed to be up-regulated and sustained on exhausted virus-specific CD8 T cells in mice infected by the lymphocytic choriomeningitis virus LCMV, as well as during infection by the human immunodeficiency virus-1 (HIV-1), the hepatitis C virus HCV, in humans and the simian immunodeficiency virus (SIV) in monkeys (Velu et al., 2009) (Day et al., 2006 Nature 443, 350-354; Freeman et al., 2006 J Exp Med 203, 2223-2227.). PD-1 expression correlates with viral load in LCMV infected mice, in HIV-infected patients and SIV-infected monkeys (Day et al., 2006 Nature 443, 350-354; Freeman et al., 2006 J Exp Med 203, 2223-2227.). Further, the in vivo blockade of PD-1-PDL1/2 binding restores the function of virus-specific CD8+ T cells, resulting in enhanced viral clearance (Ha et al 2008; J Exp Med 205, 543-555; Wherry 2011 Nat Immunol 12, 492-499). Anti-PD-1 blockade also been shown to cooperate with other therapeutic antibodies to other co-receptors such as CTLA-4 and -cell immunoglobulin domain and mucin domain 3 (Tim-3) in CD8 T-cell exhaustion during chronic viral infection (Jin et al 2010 Proc Natl Acad Sci USA 107, 14733-14738). A similar approach has been used successfully in the treatment of certain cancers such as melanoma. Particularly impressive results have been obtained with the combination of anti-PD-1 and anti-CTLA-4 or LAG-3 therapy Hodi et al., 2010 N Engl J Med 363, 711-723; Wolchok et al., 2013a N Engl J Med 369, 122-133). While PD-1 plays a central role in the development of Th1 responses involving the generation of cytolytic T-cells, it's blockade has also been reported to augment Th17 and suppress Th2 responses in peripheral blood from patients with prostate and advanced melanoma cancer (Dulos et al J Immunother. 2012 35, 169-78).

T-box transcription factor TBX21 or Tbet is a protein that in humans is encoded by the TBX21 gene (Szabo et al 2015 J Immunol. 194, 2961-75; Szabo et al 2000 Cell 100, 655-69); Lazarevic. et al 2013 Nat Rev Immunol. 13, 777-89). This gene is a member of a phylogenetically conserved family of genes that share a common DNA-binding domain, the T-box. T-box genes encode transcription factors involved in the regulation of developmental processes. This gene is the human ortholog of mouse Tbx21/Tbet gene (Szabo et al 2015 J Immunol. 194, 2961-75). Studies in mouse show that Tbx21 protein is a Th1 cell-specific transcription factor that controls the expression of the hallmark Th1 cytokine, interferon-γ (IFNγ). Expression of the human ortholog also correlates with IFNγ expression by Th1 and natural killer cells, suggesting a role for this gene in initiating Th1 lineage development from naive Th precursor cells (Lazarevic. et al 2013 Nat Rev Immunol. 13:777-89). Tbet is reportedly upregulated during some autoimmune or inflammatory conditions such as rheumatoid arthritis, inflammatory bowel disease or Crohn's disease, and during some parasitic infections that alter regulatory T cell activity. Genetic polymorphisms in Tbet have been associated with various autoimmune disorders (Sasaki et al 2004 Hum. Genet. 115 (3): 177-84; Raby et al 2006 Am. J. Respir. Crit. Care Med. 173: 64-70).

### SUMMARY

The present invention broadly relates to the use of GSK-3 modulators which modulate PD-1 and/or Tbet expression by immune cells, especially T cells in order to downregulate or upregulate T cell immunity in a subject in need thereof.

More specifically, the present invention provides methods of therapy in subjects in need thereof, which therapies comprises the administration an amount of at least GSK-3 inhibitor that modulates PD-1 expression, wherein said administration promotes T cell immunity, especially T_{H}1 or CTL immunity, by downregulating PD-1 transcription or PD-1 expression, e.g., for the treatment of a cancerous or other proliferative disorder or an infectious condition, e.g., a cancer characterized by the expression of PD-L1 or PD-L2. In preferred embodiments the therapy will include the administration of another immune modulator such as an PD-1 antagonist or CTLA-4 antagonist.

Also, the present invention provides methods of therapy in subjects in need thereof, which therapies comprises administration an amount of at least GSK-3 inhibitor that modulates T-bet expression, wherein said administration promotes T cell immunity by upregulating Tbet transcription or expression, e.g., for the treatment of a cancerous or other proliferative disorder or an infectious condition, e.g., a cancer characterized by the expression of PD-L1 or PD-L2.

Also, the present invention provides methods of therapy in subjects in need thereof, which therapies comprises administration an amount of at least GSK-3 inhibitor that modulates T-bet and/or PD-1 expression, wherein said administration is used to treat an infectious condition, e.g., caused by a bacteria, virus, yeast or other fungi or a parasite.

Also, the present invention provides in vivo or in vitro methods of inhibiting PD-1-elicited effects on immune cells comprising contacting immune cells with at least one compound that inhibits one or more of GSK-3α, GSK-3β and GSK-3β2, wherein such GSK-3 inhibitor inhibits or arrests the transcription or expression of PD-1 by immune cells or promotes the expression of Tbet by immune cells including T lymphocytes, and potentially other immune cells such as B lymphocytes, macrophages, dendritic cells, natural killer cells, mast cells, myeloid cells, or monocytes.

Also, the present invention provides methods of promoting CD4⁺ or CD8⁺ T cell immunity in a subject comprising the administration of at least one at least one compound that inhibits one or more of GSK-3α, GSK-3β and GSK-3β2, wherein such GSK-3 inhibitor inhibits or arrests the transcription or expression of PD-1 by immune cells.

Also, the present invention provides methods of promoting T_{H}1 immunity in a subject comprising the administration of at least one compound that inhibits one or more of GSK-3α, GSK-3β and GSK-3β2, wherein such GSK-3 inhibitor inhibits or arrests the transcription or expression of PD-1 by immune cells.

Also, the present invention provides methods of promoting the production of memory T cells or effector cells in a subject comprising the administration of at least one compound that inhibits one or more of GSK-3α, GSK-3β and GSK-3β2 wherein such GSK-3 inhibitor inhibits or arrests the transcription or expression of PD-1 by immune cells.

Also, the present invention provides methods of inhibiting the number, function or infiltration of T_{REG} cells in a patient in need thereof comprising the administration of at least one GSK-3α, GSK-3β or GSK-3β2 inhibitor, wherein such GSK-3 inhibitor inhibits or arrests the transcription or expression of PD-1 by immune cells.

Also, it is an object of the invention to provide a method of inhibiting the number or infiltration of T_{REG} cells for inhibiting the suppressive function of Tregs in a patient in need thereof comprising the administration of at least one GSK-3α, GSK-3β or GSK-3β 2 inhibitor, wherein such GSK-3 inhibitor inhibits or arrests the transcription or expression of PD-1 by immune cells or promotes Tbet expression by immune cells, e.g., a subject with a cancer or infectious disease.

Also, it is an object of the invention to provide a method for increasing the immunosuppressive activity of T_{REG} cells in a patient in need thereof by the administration of an activator of GSK3, wherein said activator activates at least one GSK-3α, GSK-3β and GSK-3β2, e.g. in a patient with an allergic, autoimmune or inflammatory condition.

Also, it is an object of the invention to provide a method of wherein said activator activates at least one GSK-3α, GSK-3β and GSK-3β2, e.g. a patient with an allergic, autoimmune or inflammatory condition.

Also, the present invention provides methods of therapy as above-described wherein the treated subject prior to treatment has an increased number of immune cells including T cells that express PD-1.

Also, the present invention provides methods of therapy as above-described wherein the treated subject comprises immune cells including T cells which prior to treatment are characterized by higher than normal levels of PD-1 expression.

Also, the present invention provides methods of therapy as above-described which include monitoring levels of PD-1 expression by immune cells of the treated subject before, during or after treatment.

Also, the present invention provides methods of therapy as above-described which include detecting the levels of PD-1 protein using antibodies specific thereto.

Also, the present invention provides methods of therapy as above-described which detect levels of PD-1 nucleic acids using probes specific thereto.

Also, the present invention provides methods of therapy as above-described wherein immune cells including T cells of the treated subject prior to treatment are characterized by lower than normal levels of Tbet expression.

Also, the present invention provides methods of therapy as above-described which includes monitoring levels of Tbet expression by immune cells of the treated subject before, during or after treatment.

Also, the present invention provides methods of therapy as above-described which include detecting levels of Tbet protein using antibodies specific thereto.

Also, the present invention provides methods of therapy as above-described which include detecting levels of Tbet nucleic acids using probes specific thereto.

Also, the present invention provides methods of therapy as above-described wherein the GSK-3 inhibitor is a chemical compound.

Also, the present invention provides methods of therapy as above-described wherein the GSK-3 inhibitor is selected from an antibody, an antibody fragment, anti-sense RNA, and small hairpin loop RNA (shRNA), and a small interfering RNAs (siRNA).

Also, the present invention provides methods of therapy as above-described which further includes the administration of another agent which modulates (promotes) T cell immunity.

Also, the present invention provides methods of therapy as above-described which comprise or consist of the use of a GSK3 inhibitor and another immune modulator selected from a cytokine or antagonist or agonist of a receptor or ligand expressed by an immune cells e.g., a B cell, T cell, dendritic cell, macrophage, monocyte, natural killer cell, or mast cell.

Also, the present invention provides methods of therapy as above-described which comprise or consist of the use of a GSK3 inhibitor and a PD-1 antagonist or a CTLA4 antagonist, wherein these moieties in combination elicit a synergistic or additive effect on immunity.

Also, the present invention provides methods of therapy as above-described which further include the use of another agent agonizes or antagonizes a receptor on an immune cell, e.g., a B7/CD28 or TNF receptor or ligand.

Also, the present invention provides methods of therapy as above-described which include the use of an antibody specific to a B7 or TNF/R ligand or receptor or comprises a fusion protein comprising a B7/CD28 or TNF/R receptor or ligand.

Also, the present invention provides methods of therapy as above-described which include the use of an agonist or antagonist of a receptor or ligand such as B7.1 (CD80), B7.2 (CD86), B7-DC (PD-L2 or CD273), B7-H1, B7-H2, B7-H3 (CD276), B7-H4 (VTCN1), B7-H5 (VISTA), B7-H6 (NCR3LG1), B7-H7 (HHLA2), PD-1 (CD279), PD-L3, CD28, CTLA-4 (CD152), ICOS(CD278), BTLA, NCR3, CD28H, NKp30, CD40, CD40L (CD154), LTα, LTβ, LT-βR, FASL (CD178), CD30, CD30L (CD153), CD27, CD27L (CD70), OX40, OX40L, TRAIL/APO-2L, 4-1BB, 4-1BBL, TNF, TNF-R, TNF-R2, TRANCE, TRANCE-R, GITR or “glucocorticoid-induced TNF receptor”, GITR ligand, RELT, TWEAK, FN14, TNFα, TNFβ, RANK, RANK ligand, LIGHT, HVEM, GITR, TROY, and RELT.

Also, the present invention provides methods of therapy as above-described which include the use of an agent inhibits the activity of an NK inhibitory receptor or promotes the activity of an NK activating receptor.

Also, the present invention provides methods of therapy as above-described which include the use of an agent specifically binds to PD-1, PD-L1, PD-L2, CTLA-4, LAG3, Tim3, VISTA or another modulatory receptor expressed on the surface of T-cells.

Also, the present invention provides methods of therapy as above-described which include the use of an antibody against PD-1, CTLA-4, and LAG3, Tim3, VISTA or other modulatory receptors on the surface of T-cells.

Also, the present invention provides methods of therapy as above-described which include the use of a cytokine such as IFNγ, IL-12, IL-18 or IL-21 or another agent that enhances Th1 and CTL responses and/or inhibits the development of Th2 or Th17 cells and/or which increases transcription of cytokine receptors such as IL-23R.

Also, the present invention provides methods of therapy as above-described which include the use of another agent which is an anti-PD-1, PD-L1 PD-L2, CTLA-4 antibody and the combination elicits a synergistic effect on CTL cell immunity.

Also, the present invention provides methods of therapy as above-described which include the use of an interferon, interleukin, such as IFNα, IFNβ, IFNγ, IL-12, IL-18 or IL-21.

Also, the present invention provides methods of therapy as above-described which include the use of anther agent is an antibody to CD28 or another antibody which enhances Th1 and CTL responses and/or reduces the development of Th2 or Th17 cells.

Also, the present invention provides methods of therapy as above-described which include the use of another agent increase the transcription of cytokine receptors, e.g., IL-23R.

Also, the present invention provides methods of therapy as above-described wherein the treated subject has a cancer selected from a carcinoma, lymphoma, blastoma, sarcoma, and leukemia.

Also, the present invention provides methods of therapy as above-described wherein the treated subject has a cancer selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Müllerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sézary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenström's macroglobulinemia, Warthin's tumor, Wilms' tumor, or any combination thereof.

Also, the present invention provides methods of therapy as above-described to treat a B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD), melanoma, ovarian cancer, brain cancer, solid tumors, stomach cancer, oral cancers, testicular cancer, uterine cancer, scleroderma, bladder cancer, esophageal cancer, et al.

Also, the present invention provides methods of therapy as above-described to treat a disease treated which is characterized by the increased expression of one or more immunosuppressive immune factors.

Also, the present invention provides a method of therapy in a subject in need thereof, which therapy comprises the treatment of the administration an amount of at least compound which promotes the expression and/or activation of at least one GSK-3 isoform, wherein this increases PD-1 expression, and thereby reduces T cell immunity by upegulating PD-1 transcription or expression, e.g., a subject with an autoimmune, allergic or inflammatory condition.

Also, the present invention provides a method of therapy in a subject in need thereof, which therapy comprises the treatment of the administration an amount of at least compound which promotes the expression and/or activation of at least one GSK-3 isoform, wherein this decreases Tbet expression, and thereby reduces T cell immunity by downregulating Tbet transcription or expression, e.g., a subject with an autoimmune, allergic or inflammatory condition.

Also, the present invention provides methods as above-described wherein the compound which promotes the expression and/or activation of at least one GSK-3 isoform is selected from Pyk2, Fyn, Src, Csk, octreotide, lysophosphatidic acid, leucine-rich repeat kinase 2 (LRRK2), 6-hydroxydopamine, and sphingolipids such as psychosine.

Also, the present invention provides methods as above-described which further include the administration of another compound which up regulates or agonizes PD-1, e.g., an agonistic PD-1 antibody or a PD-L1 or PD-L2 fusion protein.

Also, the present invention provides methods as above-described, in combination with antibody therapies that suppress T_{H}1 immunity.

Also, the present invention provides methods of screening for a PD-1 modulator comprising the steps of:
(i) incubating a test molecule with GSK-3;
(ii) measuring the level of GSK-3 activity in said sample; and
(iii) comparing the level of GSK-3 activity in the sample with the level of GSK-3 activity in a control sample in which the test molecule is absent; wherein a change in the level of GSK-3 activity as compared to the control is indicative of a PD-1 modulator, and further wherein a decrease in the level of GSK-3 activity is indicative of PD-1 up-regulation and an increase indicative of immune down regulation.

According to a further aspect of the invention, the invention provides methods for using GSK-3 inhibitors, including inhibitors of one or more of its isoforms: GSK-3α, GSK-3β and GSK-3β2 which inhibit PD-1 expression for inhibiting PD-1 expression by immune cells, especially T-cells in an animal or human patient in need thereof.

According to a further aspect of the invention, there is provided a pharmaceutical composition comprising a GSK-3 inhibitor that inhibits PD-1 expression and/or promotes Tbet expression by T cells and one or more pharmaceutically acceptable excipients, diluents or carriers for use in treating conditions where upregulation of T cell immunity is desirable such as for the treatment of infection and cancer.

According to a further aspect of the invention, there is provided a pharmaceutical composition comprising a GSK-3 inhibitor that promotes PD-1 expression and/or inhibits Tbet expression by T cells and one or more pharmaceutically acceptable excipients, diluents or carriers for use in the treatment of conditions wherein suppression of T cell immunity is desirable such as allergic, autoimmune or inflammatory conditions.

According to a further aspect of the invention, there is provided a method of treating infection and cancer by administering to the subject an effective amount of a GSK-3 inhibitor that modulates PD-1 expression, for use alone, or in combination with another immune modulator such as an antibody treatment to surface receptors on T-cells or a chimeric antigen receptor (CAR) or other drugs useful in the treatment of infection and cancer.

In another aspect the present invention provides synergistic therapeutic combinations comprising a GSK-3 inhibitor which inhibits PD-1 transcription or expression and another molecule which antagonizes or inhibits PD-1 or a PD-1 or PD-L2 ligand, e.g., an anti-PD-1 antibody, anti-PD-L1 antibody or anti-PD-L2 antibody wherein such combination more effectively antagonizes PD-1 than the corresponding monotherapy in treating conditions wherein PD-1 antagonism is therapeutically desirable such as cancers and infectious disorders.

### BRIEF DESCRIPTION OF THE FIGURES

FIG. 1(a, b, c, d, e, f) contain the results of in vitro experiments demonstrating that the incubation of T cells with two different GSK-3 inhibitors (SB215286 or SB216763) inhibited PD-1 transcription and expression and increased Tbet transcription in T cells. OT-1 T-cells were stimulated in vitro by OVA peptide presented by EL-4 cells. (a) FACs profile showing SB415286 down-regulation of PD-1 expression (grey background: isotype control; dark line: OVA peptide; light line: OVA peptide plus SB415286); (b) FACs profile showing SB216763 down-regulation of PD-1 expression (grey background: isotype control; dark line: OVA peptide; light line: OVA peptide plus SB216763; (c): Quantitative PCR analysis showing SB415286 and SB216763 down-regulation of PD-1 transcription; (d) Quantitative PCR showing that SB415286 and SB216763 increase Tbet transcription; (e) GSK-3 inhibition by SB415286 enhances OT-1 cytolytic killing of EL4-OVA target cells via the down-regulation of PD-1. % target killing of EL4-OVA targets by OT-1 CD8+ cytolytic T-cell (CTL) activated in the presence or absence of SB415286 with or without blocking anti-PD-1 (f) GSK-3 inhibition by SB216763 enhances OT-1 cytolytic killing of EL4-OVA target cells via the down-regulation of PD-1% target killing of EL4-OVA targets by OT-1 CD8+ cytolytic T-cell (CTL) incubated in the presence or absence of SB216763 with or without blocking anti-PD-1.

FIG. 2(a)-(f) contains the results of FACS experiments detecting the expression of T cell proteins by T cells incubated with a GSK-3 inhibitor, demonstrating that the down-regulation of PD-1 expression by GSK-3 inhibitor (SB415286) occurs without the inhibition of other T cell receptors or ligands. OT-1 T-cells were stimulated in vitro by OVA peptide presented by EL-4 cells. (a) PD-1; (b) CD3; (c) CD44.

FIG. 3(a-c) contains the results of FACS experiments detecting the expression of T cell proteins by T cells incubated with a second GSK-3 inhibitor, demonstrating that the down-regulation of PD-1 expression by GSK-3 inhibitor (SB216763) similarly occurs without the inhibition of other T cell receptors or ligands. (a) PD-1; (b) CD3; (c) FasL.

FIG. 4 shows the effects of structurally distinct competitive and non-competitive inhibitors of GSK-3 on PD-1 expression. Primary DO11.10 mouse T-cells were activated with either anti-CD3 (2C11) for 48 hours in the presence or absence of inhibitor followed by harvesting of cells and FACs analysis using anti-PD-1-PE (CD279; clone J43; Affymetrix eBioscience). FACS histogram showing PD-1 expression on T-cells and the inhibition of expression by inhibitors SB216763, SB415286, L803-mts, AR-A014418, CT99021 and the thiadiazolidinone TDZD-8. The chemical structures of each inhibitor are shown on bottom and right sides of figure.

FIG. 5(a-f) shows the effects of different GSK-3 inhibitors on PD-1 expression induced by a mixed lymphocyte reaction (MRL) (a-d) and Concanavalin A (Con A (e,f). For the MLR, inbred C57Bl/6 and outbred ICR/CD1 (Taconic labs) mouse spleen T-cells were either cultivated alone or co-cultured at equal numbers for 60 hours in the presence or absence of inhibitors AR-A014418 or CT99021 followed by FACs analysis for PD-1 expression. Splenocytes from outbred ICR/CD1 mice will mount a stronger immune response to inbred C57Bl/6 mice and vice versa. (a) Bright field images of B6 or ICR/CD1 T-cells alone or co-cultured in the absence or presence of AR-A014418 (arrow points to cell clusters); (b) FACS histogram showing PD-1 expression on T-cells (light line: control (no antibody); dark line: anti-PD-1-PE (CD279; clone J43) staining of cells cultured in MLR in the absence of the inhibitor); (c) FACS histogram showing the inhibition of PD-1 expression on T-cells by GSK-3 inhibitor AR-AO14418 in the same assay (light line: control (no antibody); dark line: anti-PD-1-PE staining of cells cultured in MLR with AR-AO14418); (d) FACS histogram showing the inhibition of PD-1 expression on T-cells by GSK-3 inhibitor CT99021 in the same assay (light line: control (no antibody); dark line: anti-PD-1-PE staining of cells cultured in MLR with C199021); (e) shows that non-ATP competitive GSK-3 inhibitor TDZD-8 inhibits PD-1 expression on Con A activated T-cells. Bright field images of T-cells alone or in co-culture (arrow points to cell clusters): (f): FACS histogram shows the % of T-cells with PD-1 expression and the inhibition of expression by TDZD-8.

FIG. 6(a-f)—shows that GSK-3 inhibition by SB215286 cooperates with anti-CTLA-4 to down-regulate PD-1 and increase cell proliferation. C57BL/6J (B6) or outbred mouse CRI/CD1 T-cells were cultivated either alone or together at equal numbers for 60 hours in the presence or absence of inhibitor followed by harvesting of cells and FACs analysis for PD-1 using anti-PD-1-PE (CD279; clone J43; Affymetrix eBioscience) (a) SB415286 reduced the expression of PD-1 on cells from B6/CRI/CD1 (C57BL/6J-CRI/CD1) cultures; (b); anti-CTLA-4 reduced the expression of PD-1 when compared the B6/CRI/CD1 control (c); anti-CTLA-4 and SB415286 individually reduced the expression of PD-1 to a similar extent (d); the combination of anti-CTLA-4/SB415286 reduced the expression of PD-1 further, greater than each individually (compare to c). (e) Bright field images of cells cultured in the presence and absence of SB415286. (f) Anti-CTLA-4+SB415286 cooperated to increase the percent of T-cell blasts.

FIG. 7(a)-(c) contains the results of in vivo experiments conducted with a mouse tumor EL-4 model (on mid-ranged aged mice 6-10 weeks) which show that the administration of a GSK-3α/β inhibitor SB415286 eliminated EL4 tumor cells. (a) Upper panel images; lower panel: histogram) concurrent with reduced PD-1 transcription (b) and increased Tbet transcription (c).

FIG. 8(a)-(c) contains the results of in vivo experiments conducted in an induced mouse tumor model (on mid aged mice: 6-10 weeks) which show that the administration of a GSK-3α/β inhibitor SB415286 eliminated tumors in a manner similar to anti-PD-1 treatment (a). Panel (b) shows a comparison of the effectiveness of SB415286 and anti-PD-1 in reducing tumor size (i.e. tumor size relative to untreated control 100%) and occurs concurrent with reduced PD-1 transcription (c) and increased Tbet transcription (d) by T cells.

FIG. 9 (a)-(d) contains the results of in vivo experiments conducted using an EL-4 tumor model (on young mice aged 4-6 weeks) which show that the administration of an GSK-3α/β inhibitor SB216763 eliminated EL4 tumor cells (a; upper panel images; lower panel: histogram) together with reduced PD-1 (b) and increased Tbet transcription (c). (d) shows the down-regulation of PD-1 expression in the T-cells (upper panel). No effect was apparent on the expression of FasL (lower panel).

FIG. 10 (a)-(c) contains the results of in vivo experiments conducted using an EL-4 tumor model (on older mice aged 6 months) which show that the administration of an GSK-3α/β inhibitor SB415286 eliminated EL4 tumor cells (a; upper panel images; lower panel: histogram) together with reduced PD-1 (b) and increased Tbet transcription (c).

FIG. 11 (a)-(f) show that anti-PD-1 cooperates with SB415286 inhibition of GSK-3 to down-regulate the expression of PD-1 on the surface of T-cells. (a) shows the expression of PD-1 on OT-1 T-cells stimulated by EL-4-OVA presentation to OT-1 T-cells in vitro (dark line: OVA); (b) shows that the presence of SB415286 reduced PD-1 expression on OVA activated OT 1 T-cells (dark line: OVA+SB415286)(see relative to a); (c) shows that anti-PD-1 cooperates with SB415286 to reduce PD-1 expression on OVA activated OT 1 T-cells (dark line: OVA+SB415286+anti-PD-1)(see relative to b); (d) Quantitative PCR analysis showing SB415286 synergizes with anti-PD-1 to inhibit PD-1 transcription; (e) Further examples of anti-PD-1 inhibition of its own transcription on T-cells (two additional experiments). (f) shows the down-regulation of PD-1 due to anti-PD-1 ligation as seen by FACs staining with anti-PD-1-PE. The results show that PD-1 expression and transcription is inhibited by the GSK-3 inhibitors and by the anti-PD-1 antibody and importantly, they cooperate to maximally suppress PD-1 transcription.

FIG. 12(a)-(f) shows that the drug induced in vivo down-regulation of PD-1 and tumor elimination was accompanied by an increase in the expression of Interferon-γ1, (IFN-γ1) a key component in CD8+ CTL killing. (a) shows the down-regulation of PD-1 by SB216763; (b) shows the increase expression of IFN-γ1; (c) shows an second experiment where IFN-γ1 levels are increased by inhibition of GSK-3 using SB216763; (d) shows a possible minor increase in CD69 expression; (e) shows the expression of CTLA-4 in the presence of SB216763; (f) shows a histogram representation of the % max intensity of IFN-γ1 due to PD-1 down-regulation and GSK-3 inhibition.

FIG. 13(a)-(e) shows that the oral administration in vivo inhibits PD-1 expression. (a) Histogram showing the regime of oral drug administration. Mice were feed TDZD-8 orally in the water. (b) Bright-field mages of T-cells in culture from the ocular; (c) Cell numbers in culture following ex vivo culturing of cells; (d) FACs profile showing a reduction in PD-1 expression on ex vivo cells from mice that had been given the drug TDZD-8 orally; (e) Histogram showing that the in vivo oral administration of TDZD-8 reduce the percentage of T-cells expressing PD-1. These data show that an inhibitor of GSK-3 can be administered orally to achieve the down-regulation of PD-1.

### DETAILED DESCRIPTION

The present invention broadly relates to the discoveries that GSK-3 controls PD-1 transcription by immune cells, e.g., T-cells, and increases Tbet expression by T cells and that based on these discoveries that GSK-3 inhibitors may be used as immune modulators in order to inhibit or arrest the expression of PD-1 by T cells and thereby promote T cell immunity, especially TH1, CD4⁺ and CD8⁺ T cell immunity.

Also, based on the discoveries that GSK-3 controls PD-1 transcription by immune cells, e.g., T-cells, and increase Tbet expression the invention further relates to the use of GSK-3 activators which induce PD-1 transcription or expression and/or which suppress Tbet as immune modulators in order to promote or upregulate the expression of PD-1 by T cells and decrease Tbet and thereby suppress T cell immunity in a subject in need thereof.

The present discoveries have therapeutic application in the treatment of various conditions wherein enhanced T cell immunity is therapeutically desirable such as in the treatment of cancer, and infectious disease. Also, these discoveries have therapeutic application in the treatment of various conditions wherein the suppression of T cell activation or aberrant T cell activity is therapeutically desirable such as in the treatment of allergy, autoimmunity or inflammation.

However, before describing the invention in more detail the following definitions are provided. Otherwise all words and phrases herein are to be accorded their usual definition, as construed by a skilled artisan.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims. As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

“Agonist” refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors (e.g., a PD-1 agonist or a TNF superfamily member or B7 superfamily member agonist).

“Antagonist” refers to a compound that when contacted with a molecule of interest, e.g. a TNF or TNFR family superfamily member or other ligand or receptor causes a decrease in the magnitude of a certain activity or function of the molecule compared to the magnitude of the activity or function observed in the absence of the antagonist. Particular antagonists of interest herein include PD-1 and other T cell receptor agonists or antagonists that promote T cell immunity such as anti-CTLA4 and anti-PD-1 antibodies.

“Antigen” refers to any substance that is capable of being the target of an immune response. An antigen may be the target of, for example, a cell-mediated and/or humoral immune response raised by a subject organism. Alternatively, an antigen may be the target of a cellular immune response (e.g., immune cell maturation, production of cytokines, production of antibodies, etc.) when contacted with immune cells.

“GSK-3 inhibitor” according to the present invention includes any GSK-3 inhibitor which inhibits the activity of any GSK-3 isoform, wherein such inhibition inhibits the transcription or expression of PD- by T cells and/or increases the expression of Tbet by T or other immune cells in vitro and/or in vivo. Therefore, the term “GSK-3 inhibitor” potentially includes any compound which inhibits one or more (generally, all to a greater or lesser degree) of GSK-3α, GSK-3 β and/or GSK-3β2, in particular GSK-3β, wherein such inhibition inhibits the transcription or expression of PD- by T cells and/or increase the expression of Tbet by immune cells in vitro and/or in vivo. As shown in the examples infra, in vitro or in vivo assays may be conducted in order to detect whether a particular GSK-3 inhibitor inhibits PD-1 transcription or expression and/or increases Tbet transcription and expression by immune cells, especially T or other PD-1 or Tbet expressing immune cells. With respect thereto, this application demonstrates with 2 different GSK-3 inhibitors that these compounds both inhibited PD-1 and T bet expression by immune (T) cells. Based thereon, it is anticipated that other GSK-3 inhibitory compounds will inhibit PD-1 and/or increase Tbet expression.

Accordingly, a GSK-3 inhibitor herein includes any compound which inhibits the transcription or expression of GSK-3 α and/or GSK-β, or other GSK-3 isoform and/or which inhibits the activity of GSK-α and/or GSK-3 β, wherein such inhibitory compound further increases the transcription or expression of Tbet or decreases the transcription or expression of PD-1 by immune cells in vivo or in vitro, and in particular which inhibits transcription or expression of PD-1 by T cells.

Examples of GSK-3 inhibitory compounds potentially useful in the present invention are disclosed infra and further include any of the GSK-3 inhibitors disclosed in U.S. Pat. No. 6,057,117, U.S. Pat. No. 6,153,618; U.S. Pat. No. 6,417,185; U.S. Pat. No. 6,441,053; U.S. Pat. No. 6,489,344; U.S. Pat. No. 6,465,231; U.S. Pat. No. 6,608,063; U.S. Pat. No. 6,610,677; U.S. Pat. No. 6,638,926; U.S. Pat. No. 6,653,300; U.S. Pat. No. 6,653,301; U.S. Pat. No. 6,656,939; U.S. Pat. No. 6,660,731; U.S. Pat. No. 6,664,247; U.S. Pat. No. 6,689,452; U.S. Pat. No. 6,716,624; U.S. Pat. No. 6,743,791; U.S. Pat. No. 6,747,057; U.S. Pat. No. 6,756,385; U.S. Pat. No. 6,762,179; U.S. Pat. No. 6,780,625; U.S. Pat. No. 6,800,874; U.S. Pat. No. 6,825,190; U.S. Pat. No. 6,872,737; U.S. Pat. No. 6,989,385; U.S. Pat. No. 6,916,798; U.S. Pat. No. 7,008,948; U.S. Pat. No. 7,037,918; U.S. Pat. No. 7,045,519; U.S. Pat. No. 7,056,939; U.S. Pat. No. 7,062,219; U.S. Pat. No. 7,078,410; U.S. Pat. No. 7,091,343; U.S. Pat. No. 7,098,330; U.S. Pat. No. 7,101,848; U.S. Pat. No. 7,105,532; U.S. Pat. No. 7,115,739; U.S. Pat. No. 7,135,321; U.S. Pat. No. 7,157,422; U.S. Pat. No. 7,195,886; U.S. Pat. No. 7,217,712; U.S. Pat. No. 7,244,735; U.S. Pat. No. 7,250,443; U.S. Pat. No. 7,256,190; U.S. Pat. No. 7,259,022; U.S. Pat. No. 7,262,200; U.S. Pat. No. 7,268,136; U.S. Pat. No. 7,300,944; U.S. Pat. No. 7,300,943; U.S. Pat. No. 7,348,308; U.S. Pat. No. 7,361,484; U.S. Pat. No. 7,378,111; U.S. Pat. No. 7,378,432; U.S. Pat. No. 7,390,808; U.S. Pat. No. 7,390,815; U.S. Pat. No. 7,405,305; U.S. Pat. No. 7,425,557; U.S. Pat. No. 7,446,092; U.S. Pat. No. 7,446,199; U.S. Pat. No. 7,452,887; U.S. Pat. No. 7,456,190; U.S. Pat. No. 7,462,621; U.S. Pat. No. 7,465,737; U.S. Pat. No. 7,488,727; U.S. Pat. No. 7,452,873; U.S. Pat. No. 7,491,730; U.S. Pat. No. 7,507,743; U.S. Pat. No. 7,514,445; U.S. Pat. No. 7,531,536; U.S. Pat. No. 7,531,561; U.S. Pat. No. 7,547,705; U.S. Pat. No. 7,585,853; U.S. Pat. No. 7,598,288; U.S. Pat. No. 7,582,630; U.S. Pat. No. 7,563,584; U.S. Pat. No. 7,566,720; U.S. Pat. No. 7,572,949; U.S. Pat. No. 7,582,630; U.S. Pat. No. 7,585,853; U.S. Pat. No. 7,589,232; U.S. Pat. No. 7,595,319; U.S. Pat. No. 7,598,288; U.S. Pat. No. 7,598,632; U.S. Pat. No. 7,666,647; U.S. Pat. No. 7,671,049; U.S. Pat. No. 7,671,072; U.S. Pat. No. 7,695,926; U.S. Pat. No. 7,683,067; U.S. Pat. No. 7,700,609; U.S. Pat. No. 7,709,473; U.S. Pat. No. 7,723,301; U.S. Pat. No. 7,732,151; U.S. Pat. No. 7,781,440; U.S. Pat. No. 7,807,430; U.S. Pat. No. 7,833,974; U.S. Pat. No. 7,883,881; U.S. Pat. No. 7,850,960; U.S. Pat. No. 7,872,129; U.S. Pat. No. 7,935,493; U.S. Pat. No. 7,947,851; U.S. Pat. No. 8,017,619; U.S. Pat. No. 8,048,454; U.S. Pat. No. 8,063,221; U.S. Pat. No. 8,071,591; U.S. Pat. No. 8,088,941; U.S. Pat. No. 8,148,094; U.S. Pat. No. 8,158,661; U.S. Pat. No. 8,187,878; U.S. Pat. No. 8,198,037; U.S. Pat. No. 8,207,216; U.S. Pat. No. 8,211,428; U.S. Pat. No. 8,288,400; U.S. Pat. No. 8,318,467; U.S. Pat. No. 8,318,476; U.S. Pat. No. 8,323,919; U.S. Pat. No. 8,349,822; U.S. Pat. No. 8,367,351; U.S. Pat. No. 8,389,514; U.S. Pat. No. 8,426,425; U.S. Pat. No. 8,431,395; U.S. Pat. No. 8,455,648; U.S. Pat. No. 8,497,080; U.S. Pat. No. 8,563,309; U.S. Pat. No. 8,476,621; U.S. Pat. No. 8,592,436; U.S. Pat. No. 8,592,437; U.S. Pat. No. 8,592,485; U.S. Pat. No. 8,598,175; U.S. Pat. No. 8,598,187; U.S. Pat. No. 8,628,931; U.S. Pat. No. 8,653,088; U.S. Pat. No. 8,663,633; U.S. Pat. No. 8,664,246; and U.S. Pat. No. 8,669,081 the contents of which are incorporated by reference in their entireties herein.

“GSK-3 activator” according to the present invention includes any compound which promotes the expression or the activation of any GSK-3 isoform, wherein such activation promotes the transcription or expression of PD-1 by T cells and/or decreases the expression of Tbet in vitro and/or in vivo. Therefore, the term “GSK-3 activator” potentially includes any compound which promotes the expression or activation of one or more (generally, all to a greater or lesser degree) of GSK-3α, GSK-3 β and/or GSK-3β2, in particular GSK-3β, wherein such activation or increase in expression promotes the transcription or expression of PD-1 by T cells and/or decreases the expression of Tbet by immune cells in vitro and/or in vivo. For example, GSK-3 can also be activated by tyrosine phosphorylation, such as by Pyk2, Fyn, Src, Csk, octreotide, and lysophosphatidic acid, leucine-rich repeat kinase 2 (LRRK2), 6-hydroxydopamine, sphingolipids such as psychosine,

“T-box transcription factor” or “Tbet” or TBET; T-PET; or TBLYM is the central mediator of Th1 development. This polypeptide is encoded by a gene TBX21 or T-box 21 which is a member of a phylogenetically conserved family of genes that share a common DNA-binding domain, the T-box. T-box genes encode transcription factors involved in the regulation of developmental processes. This gene is the human ortholog of mouse Tbx21/Tbet gene. Studies in mice show that Tbx21 protein is a Th1 cell-specific transcription factor that controls the expression of the hallmark Th1 cytokine, interferon-γ (IFNγ). Expression of the human ortholog also correlates with IFNγ expression in Th1 and natural killer cells, suggesting a role for this gene in initiating Th1 lineage development from naive Th precursor cells.

“TNF/R” herein generally refers to any member of either the Tumor Necrosis Factor (TNF) Superfamily or the Tumor Necrosis Factor Receptor (TNFR) Superfamily. The TNF and TNFR Superfamily includes, for example, as CD40, CD40L (CD154), LTα, LTβ, LT-13R, FASL (CD178), CD30, CD30L (CD153), CD27, CD27L (CD70), OX40, OX40L, TRAIL/APO-2L, 4-1BB, 4-1BBL, TNF, TNF-R, TNF-R2, TRANCE, TRANCE-R, GITR or “glucocorticoid-induced TNF receptor”, GITR ligand, RELT, TWEAK, FN14, TNFα, TNFβ, RANK, RANK ligand, LIGHT, HVEM, GITR, TROY, and RELT. Unless otherwise indicated, reference to a TNF/R agonist or antagonist compound can include the compound in any pharmaceutically acceptable form.

“B7 family member” or “B7-CD28 family member” refers to a member of a large family of receptors and ligands expressed on immune cells involved in immune signaling. The typical structural elements common to members of the B7 polypeptide family include an extracellular domain including a V-like and a C-like Ig domain. A signal sequence is found at the N-terminus of full-length B7 family polypeptides, and is followed, in N-to-C order, by a V-like Ig domain, a C-like Ig domain, a transmembrane domain, and an intracellular domain. There are certain key residues within the extracellular domains of B7 polypeptides, the two pairs of conserved cysteine residues—one pair in each Ig domain—that are involved in disulfide bond formation and the three-dimensional conformation of the polypeptide. The B7 polypeptide family is moderately conserved, with the Ig domains of human family members very similar to each other, and to the Ig domains of B7 family members from other species. The family includes subfamilies including B7-1 (CD80), B7-2 (CD86), and B7-H1, and the butyrophilin (BTN)/MOG (myelin oligodendrocyte glycoprotein-like) family members, with the immunomodulatory B7 subfamily lacking a B30.2 domain and the butyrophilin/MOG subfamily having a B30.2 domain. Members of the B7/CD28 superfamily include by way of example B7.1 (CD80), B7.2 (CD86), B7-DC (PD-L2 or CD273), B7-H1, B7-H2, B7-H3 (CD276), B7-H4 (VTCN1), B7-H5 (VISTA), B7-H6 (NCR3LG1), B7-H7 (HHLA2), PD-1 (CD279), PD-L3, CD28, CTLA-4 (CD152), ICOS(CD278), BTLA, NCR3, CD28H, and NKp30.

The terms “biological effects associated with X” and “X activity” e.g., a TNF or TNFR superfamily member or other immune cell receptor are used interchangeably and include any biological effect associated with the moiety with which the agonist or antagonist specifically interacts, e.g., a TNF or TNF/R superfamily member.

The term “fusion protein” refers to a molecule comprising two or more proteins or fragments thereof linked by a covalent bond via their individual peptide backbones, most preferably generated through genetic expression of a polynucleotide molecule encoding those proteins.

The term “immunoglobulin fusion protein” refers to a fusion of a functional portion of a polypeptide (generally comprising the extracellular domain of a cell surface protein) with one or more portions of an immunoglobulin constant region, e.g. the hinge, CH1, CH2 or CH3 domains or portions or combinations thereof.

Thus, the subject invention in part relates to the use of GSK-3 inhibitors which inhibit PD-1 transcription or expression and/or increase Tbet transcription or expression to promote cellular immune responses, e.g., T_{H}1 or CD4⁺ or CD8⁺ cytoxic immunity in conditions where therapeutically desired, most particularly cancer and infectious conditions.

Accordingly, this invention further relates to the discovery that activators of glycogen synthase kinase 3 (“GSK-3”) which increase PD-1 expression and/or decrease Tbet expression may be used to treat any condition wherein the promotion of PD-1 expression or suppression of Tbet is therapeutically desired, e.g., as in the treatment of autoimmunity, inflammation or allergy.

Additionally, this invention provides a means for selection of inhibitors of glycogen synthase kinase 3 (“GSK-3”) which are potentially useful in the treatment of cancer or infectious conditions based on their ability to suppress PD-1 transcription or expression and/or promote Tbet expression.

Further, this invention provides methods of using compounds that inhibit one or more isoforms of GSK-3, e.g., GSK-3α, GSK-3β and GSK-3β2, that inhibit PD-1 and/or increase Tbet expression by immune cells, e.g., T-cells, but potentially other immune cells such as B cells, macrophages, dendritic cells, myeloid cells, monocytes, natural killer cells, mast cells in order to increase cellular immunity in a human or animal subject, e.g., a subject with a neoplastic or infectious condition, e.g., one caused by a virus, bacteria, yeast or other fungi, nematode, or other parasite.

Particularly, GSK-3 inhibitors which inhibit PD-1 and/or increase Tbet, may be used to promote T_{H}1 immunity, or cytotoxic CD4⁺ and CD8⁺ T-cell mediated immunity in subjects in need thereof. This discovery is of great therapeutic potential as peripheral CD4⁺ and CD8⁺ T-cells respond to peptide antigen presented by antigen-presenting cells (APCs) such as dendritic cells (DCs) by proliferating and producing cytokines as well as developing into effector and memory T-cells (Williams and Bevan, 2007). CD4 positive cells can be divided into subsets based on their cytokine production profiles. This includes such as T-helper 1 (Th1), T-helper 2 cells (Th2) and T-helper 17 (Th17) cells. CD8 positive T-cells develop into cytolytic T-cells (CTLs) that can identify antigens for the clearance of viral infections (Williams and Bevan, 2007).

Further, persistent or chronic infections are associated with functional exhaustion of virus-specific CD8+ T cells (Day et al., 2006; Klenerman and Hill, 2005; Sarris et al., 2008; Wherry and Ahmed, 2004). This decrease in the proliferative potential of virus-specific CD8+ T cells may explain the inefficient responses of certain therapeutic vaccines (Dikici et al., 2003; Maini et al., 1999; Nisii et al., 2006; von Herrath et al., 2000; Wherry et al., 2003.). In this context, functional exhaustion is associated with the expression of inhibitory receptor programmed death 1 (PD-1; also known as PDCD1) on exhausted virus-specific CD8 T cells in mice (Ahmed et al., 2009; Ishida et al., 1992; Sharpe et al., 2007; Steinmetz et al., 2009). PD-1 is a negative regulator of activated T cell activation and function (Greenwald et al., 2005; Sharpe and Freeman, 2002). The in vivo blockade of PD-1 restores the function of virus-specific CD8+ T cells, resulting in enhanced viral clearance. Virus-specific CD8+ T cells also up-regulate PD-1 expression during chronic infections such as HIV, HCV, (Day et al., 2006) and HBV in humans 21 and SIV in monkeys (Keir et al., 2008; Sharpe and Freeman, 2002). Blocking the interaction between PD-1 and its ligands in vitro partially restored effector function and improved the proliferative capacity of exhausted CD8+ T cells in these chronic infections (Freeman et al., 2006; Kamphorst and Ahmed, 2013; West et al., 2013). Collectively, these data suggest that PD-1 signaling in T cells is a major inhibitory pathway operating during chronic infection and that its blockade in vivo may be useful for the treatment of chronic viral infections. There is therefore a need for effective treatment of chronic, prolonged diseases that result in T cell dysfunction.

In this context, ligation of the antigen receptor (T-cell receptor) induces signaling events that activate T-cells to proliferate and differentiate into CTLs. This process involves a combination of protein tyrosine and serine/threonine kinase phosphorylation cascades (Rudd, 1999; Samelson, 2002; Weiss and Littman, 1994). One pathway involves phosphoinositide dependent kinase-1 (PDK1) regulation of protein kinase B (PKB, AKT) that phosphorylates and inhibits the activity another serine-threonine kinase termed glycogen synthase kinase 3α/β (GSK-3)(Ali et al., 2001; Frame and Cohen, 2001). Various isoforms include GSK-3α, GSK-3β and GSK-3β2. In resting cells, GSK-3 is constitutively active where it acts on substrates such as NFAT, p53 and mTORCA in T-cells and other cell types (Hooper et al., 2008). GSK-3 facilitates NFAT exit from the nucleus, and inhibits its binding to DNA by phosphorylation (Beals et al., 1997; Neal and Clipstone, 2001; Ohteki et al., 2000).

Also, since T-box transcription factor (Tbet) is the central mediator of Th1 development and is therefore an important focus for drugs targeted to the immune system. This gene in humans is the ortholog of mouse Tbx21/Tbet gene (Faedo A, Ficara F, Ghiani M, et al. (2003). “Developmental expression of the T-box transcription factor T-bet/Tbx21 during mouse embryogenesis Mech. Dev. 116 (1-2): 157-60.) Tbx21 protein is a Th1 cell-specific transcription factor that controls the development and differentiation of the Th1 subset and the Th1 cytokine, interferon-γ (IFN¥). Expression of the human ortholog also correlates with IFNγ expression in Th1 and natural killer cells, suggesting a role for this gene in initiating Th1 lineage development from naive Th precursor cells. Tbet has been identified as a susceptibility gene for type 1 diabetes (Sasaki et al., 2004) as well as in asthma (Atayar et al., 2005; Chung et al., 2003; Raby et al., 2006; Tantisira et al., 2004). Its expression or increased expression has also been connected to T-cell cancers (Dorfman et al., 2005), rheumatoid arthritis, helminth infections that later T cell immunity, IBD, Crohn's disease, while HIV-1 Tat can also modulate T-bet expression and induces Th1 type of immune response (Kulkarni et al., 2005).

The data disclosed herein, describes the novel connection from the inhibition of Glycogen Synthase Kinase 3 (GSK-3) to the increase in Tbet transcription expression. This is distinct from T Helper 17 (Th17) cells that have previously been connected to GSK-3 (for example, see Beurel et al., J. Immunol. 186, 1391 (2011)).

Despite the critical function of PD-1 on T cells, the direct upstream signaling event that controls the expression of PD-1 was not known prior to the present invention. Herein, it is shown for the first time that GSK-3α/β operates upstream to control the transcription of PD-1 in CD8⁺ T cells. Inhibition or siRNA/shRNAi knock down of GSK-3 with various drugs markedly inhibited PD-1 transcription and expression on the surface of CD8⁺ T cells, while having no effect on other surface receptors examined, commensurate with enhanced CTL responses to antigen and tumors.

The experiments and data disclosed herein further show for the first time that a drug or small interfering RNA (siRNA) or small hairpin RNAs (shRNAs) which inhibits GSK-3, which inhibits the transcription and expression of the surface receptor PD-1 on cells, most especially immune cells such as T cells, may be used alone or in combination with other therapeutic agents to treat conditions wherein enhanced cellular immunity, and especially enhanced T cell immunity, is desired such as cancer and infectious conditions. The administration of GSK-3 inhibitors which inhibit PD-1 and/or Tbet, should result in enhanced in vitro and in vivo T-cell responses such as an increase in the ability of T-cells to mediate cytolytic T-cell killing of targets or the elimination of tumor or infected cells.

Moreover the inventive discovery has huge application in promoting cellular immunity against bacterially, virally, fungally or parasite infected cells. For example, the subject inhibitors may be used to treat parasite infections such as malaria, schistosomiasis, or other plasmodial parasites by the down-modulation of PD-1.

As noted previously literally many thousands of GSK-3 inhibitors have been reported in the literature. The present invention is intended to embrace the use of any GSK-3 inhibitor which effectively inhibits the transcription or expression of PD-1 and/or increases the transcription of Tbet by immune cells, especially T cells. Accordingly, and as previously mentioned, a “GSK-3 inhibitor” or “glycogen synthase kinase-3 inhibitor” useful in the invention refers to any compound or ligand capable of inhibiting one or more GSK-3 enzymes. Thus, a GSK-3 inhibitor according to the invention which inhibits PD-1 and/or increase the expression of Tbet can inhibit one member, several members or all members of the family of GSK-3 enzymes. The family of GSK-3 enzymes is well known and includes, but is not limited to, GSK-3α, GSK-3β and GSK-3β2.

GSK-3 was originally identified by virtue of its ability to phosphorylate and inactivate glycogen synthase, the rate-limiting enzyme in glycogen synthesis (Ali et al., 2001). However, it is now apparent that GSK-3 has many putative targets, including IRS-I, the translation initiation factor elF2B, transcription factors c-jun, CREB, NFAT, β-catenin, C/EBPK and the neuronal microtubule associated proteins MAP-IB and Tau (Cohen and Frame, 2001). A variety of extracellular stimuli indirectly inhibit cellular GSK-3 activity, including insulin, growth factors, Wnt cell specific proteins and cell adhesion. Since these stimuli elicit a diverse range of responses in a number of different cell types, inhibition of GSK-3 activity is potentially pivotal in mediating pleiotropic cellular responses to external stimuli. However, the potential role of GSK-3 inhibition in any given response is complicated by the fact that stimuli often initiate additional signaling pathways to the one that affects GSK-3 activity.

Therefore, in order to more definitively implicate GSK-3 inhibition in a response, it is necessary to selectively inhibit this kinase and assess whether this alone is sufficient to induce the response. Three isoforms of GSK-3 are particularly relevant to the present invention, namely GSK-3α, GSK-3β and/or GSK-3β2. Inhibitors of these enzymes and in particular, inhibitors of GSK-3β, may be used in embodiments of the invention described herein.

In some embodiments the GSK-3 inhibitor is a chemical compound or an antisense RNA, siRNA, or shRNA. In exemplary embodiments, the chemical compound is SB216763 or SB415286. In other embodiments, the GSK-3 inhibitor may comprise an antibody or an antibody fragment.

Additional GSK-3 inhibitor compounds which may be used in the present invention have been previously identified and further may include 2-arylaminopyrimidine compounds which are described and set forth in United States patent application publication US 2004/0106574 and hetero-arylamine compounds (GSK-3β inhibitors) set forth in United patent application publication US 2005/0004125. Additional references include, for example, U.S. Pat. No. 7,045,519 to Nuss, et al., U.S. Pat. Nos. 7,053,097; 7,037,918; 6,989,382; 6,960,600; 6,949,547; 6,872,737; 6,800,632; 6,780,625; 6,608,063; 6,489,344; 6,479,490; 6,441,053; 6,417,085; 6,153,618 and 6,057,147 that are directed to GSK-3 inhibitors. GSK-3 inhibitor compounds further include those described in United States patent application publication no. US 2005/0004125.

Other examples of GSK-3 inhibitors are described in, for example, WO 99/65897 and WO 03/074072 and references cited therein. For example, various GSK-3 inhibitory compounds and methods of their synthesis and use are disclosed in U.S. and international patent application Publication Nos. US 2003/0008866, US 2001/0044436 and WO 01/44246 (bicyclic based compounds); US 2001/0034051 (pyrazine based compounds); US 2002/0156087, WO 02/20495 and WO 99/65897 (pyrimidine and pyridine based compounds) and WO 98/16528 (purine based compounds). Further GSK-3 inhibitory compounds include those disclosed in WO 02/22598 (quinolinone based compounds). Further GSK-3 inhibitory compounds include macrocyclic maleimide selective GSK-3β inhibitors developed by Johnson & Johnson and described in, for example in (Kuo et al., 2003). The Pharmaprojects database indicates further GSK-3 inhibitors that are being developed by the following companies: Cyclacel (UK), Xcellsyz (UK)-XD-4241, Vertex Pharmaceuticals (USA) such as VX-608, Chiron (USA) i.e. CHIR-73911, Kinetek (Canada) i.e. KP-354.

Still further, a number of GSK-3 variants have also been described (see e.g. Schaffer et al., Gene 302, 73 (2003)). In one embodiment, the GSK-3 inhibitor is a GSK-3α, GSK-3β or GSK-3β2 inhibitor. In a further embodiment, the GSK-3 inhibitor is a GSK-3β inhibitor. GSK-3α inhibitors are also suitable as well as inhibitors for use in the invention that inhibit both isoforms of the kinase. A wide range of GSK-3 inhibitors are known, including but not limited to, the inhibitors: hymenialdisine, flavopiridol, kenpaullone, alsterpaullone, azakenpaullone, indirubin-3′-oxime, 6-bromoindirubin-3′-oxime, 6-Bromoindirubin-3′-acetoxime, aloisine A, aloisine B, CHIR 98014, CHIR 99021, ARA014418, CGP60474, TWSI 19, SU9516, CT20026, TDZD-8, SB216763 and SB415286. Other inhibitors are known and may be useful in the invention. In addition, the structure of the active site of GSK-3β has been characterized and key residues that interact with specific and non-specific inhibitors have been identified (Bertrand et al., J. Mol. Biol. 333, 393 (2003)). This structural characterization allows additional GSK-3 inhibitors to be readily identified.

GSK-3 inhibition or down-regulation of either or all isoforms can also be achieved using RNA mediated interference (RNAi) technology. Typically, double-stranded RNA molecule complementary to all or part of a GSK3 gene may be introduced into stem cells to promote the specific degradation of GSK-3-encoding mRNA molecules. This post-transcriptional mechanism of degradation results in reduced or can abolish the expression of the targeted GSK-3 gene. Suitable techniques and protocols for achieving GSK-3 inhibition using RNAi are well known to those skilled in the art. These include the use of small interfering RNAs, a class of double-stranded RNA molecules, 17-25 base pairs in length, as well as short hairpin loop RNAs, a sequence of RNA that makes a tight hairpin turn that can be used to silence target gene expression via RNA interference.

### Use of GSK-3 Inhibitors to Treat Cancer and Other Proliferative Disorders

The present invention in particular contemplates the use of GSK-3 inhibitors which inhibit PD-1 transcription and expression and/or which promote Tbet transcription and expression in vitro or in vivo by immune cells such as T cells for the treatment of cancerous and other proliferative conditions wherein suppression of PD-1 and/or increased Tbet and enhanced cellular immunity or T_{H}1 or CD4⁺ or CD8⁺ T cells or cytoxic T cell immunity is therapeutically desired.

Examples of cancers treatable by the present invention include carcinoma, lymphoma, blastoma, sarcoma, and leukemia. More particular examples of such cancers include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Müllerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sézary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenström's macroglobulinemia, Warthin's tumor, Wilms' tumor, or any combination thereof.

The present invention in particular may be used to treat s B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD), melanoma, ovarian cancer, brain cancer, solid tumors, stomach cancer, oral cancers, testicular cancer, uterine cancer, scleroderma, bladder cancer, esophageal cancer, et al.

Other preferred cancers especially amenable for treatment according to the present invention include, but are not limited to, carcinoma, blastoma, sarcoma, and leukemia or lymphoid tumors and myeloma, melanoma, lymphomas, leukemias, ovarian cancer, breast cancer, lung cancer such as non-small lung cancer (NSLC), small cell lung cancer, mesothelioma, pancreatic cancer, head and neck cancer, brain cancer, solid tumors, colorectal cancer, stomach cancer, oral cancers, testicular cancer, uterine cancer, scleroderma, bladder cancer, esophageal cancer, colorectal cancer, rectal cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma.

The cancer treated may be an early or advanced stage (including metastatic). The cancerous conditions amenable for treatment of the invention further include metastatic cancers wherein expression by myeloid derived suppressor cells suppresses antitumor responses and anti-invasive immune responses and cancers which may or may not express PD-1 ligands such as PD-L1 or PD-L2 and/or may express other immunosuppressive factors. The present invention should be particularly suitable for the treatment of vascularized or solid tumors.

GSK-3 inhibitors, e.g., siRNA's or shRNA's, small molecules or antibodies may be used as a monotherapy but more typically will be used in therapeutic regimens that include other active agents, e.g., other immune modulators or chemotherapeutic or anti-neoplastic agents. In a preferred embodiment the subject GSK-3 inhibitors will be used in a therapeutic regimen that includes the administration of another immune modulator such as a cytokine, receptor agonist or antagonist, e.g., an agonist or antagonist of a T cell receptor such as a member of the B7/CD28 or TNF/R superfamily, a TLR agonist, and the like. Examples thereof include combined therapy with anti-CTLA-4, CTLA-4Ig, anti-PD1, anti-PD-L1, anti-PD-L2, LAG3, anti-Tim3, CD40 agonists such as CD40 agonistic antibodies or CD40L, 4-1BB agonists, CD27 agonists, B7.1 or B7.2 agonists, and the like. Cytokines which may be combined with the subject GSK-3 inhibitors include interferons, interleukins, tumor necrosis factors, lymphotoxins, colony stimulating factors such as a interferon, β interferon, γ interferon, tumor necrosis factor γ, lymphotoxin, colony stimulating factor, and interleukins such as IL-2, IL-4, IL-12, IL-13, and others.

In a preferred embodiment a GSK-3 inhibitor which inhibits PD-1 expression will be used in a therapeutic regimen that includes the administration of another compound that antagonizes PD-1 such as antagonistic anti-PD-1 antibodies and antibody fragments or an anti-PD-L1 or anti-PD-L2 antibody or antibody fragment, preferably humanized or human antibodies.

Also, the subject GSK-3 inhibitors may be combined with other compounds and antibodies useful in treating the particular cancer such as chemotherapeutic, anti-angiogenesis compounds, radionuclides and radiation therapy, growth factor antagonists, hormone antagonists and the like.

Further, the subject inhibitors may be included in therapeutic regimen that includes the administration of an antigen specific to target cells such as tumor or cancerous cells.

Other active agents which may be used in cancer regimens which may be used in the inventive methods may include analgesic, antipyretic, anti-inflammatory, antibiotic, antiviral, and anti-cytokine agents. Active agents include agonists, antagonists, and modulators of TNF-.α., IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors. Active agents also include 2-Arylpropionic acids, Aceclofenac, Acemetacin, Acetylsalicylic acid (Aspirin), Alclofenac, Alminoprofen, Amoxiprin, Ampyrone, Arylalkanoic acids, Azapropazone, Benorylate/Benorilate, Benoxaprofen, Bromfenac, Carprofen, Celecoxib, Choline magnesium salicylate, Clofezone, COX-2 inhibitors, Dexibuprofen, Dexketoprofen, Diclofenac, Diflunisal, Droxicam, Ethenzamide, Etodolac, Etoricoxib, Faislamine, fenamic acids, Fenbufen, Fenoprofen, Flufenamic acid, Flunoxaprofen, Flurbiprofen, Ibuprofen, lbuproxam, Indometacin, Indoprofen, Kebuzone, Ketoprofen, Ketorolac, Lornoxicam, Loxoprofen, Lumiracoxib, Magnesium salicylate, Meclofenamic acid, Mefenamic acid, Meloxicam, Metamizole, Methyl salicylate, Mofebutazone, Nabumetone, Naproxen, N-Arylanthranilic acids, Oxametacin, Oxaprozin, Oxicams, Oxyphenbutazone, Parecoxib, Phenazone, Phenylbutazone, Phenylbutazone, Piroxicam, Pirprofen, profens, Proglumetacin, Pyrazolidine derivatives, Rofecoxib, Salicyl salicylate, Salicylamide, Salicylates, Sulfinpyrazone, Sulindac, Suprofen, Tenoxicam, Tiaprofenic acid, Tolfenamic acid, Tolmetin, and Valdecoxib. Antibiotics include Amikacin, Aminoglycosides, Amoxicillin, Ampicillin, Ansamycin, Arsphenamine, Azithromycin, Azlocillin, Aztreonam, Bacitracin, Carbacephem, Carbapenems, Carbenicillin, Cefaclor, Cefadroxil, Cephalexin, Cephalothin, Cephalothin, Cefamandole, Cefazolin, Cefdinir, Cefditoren, Cefepime, Cefixime, Cefoperazone, Cefotaxime, Cefoxitin, Cefpodoxime, Cefprozil, Ceftazidime, Ceftibuten, Ceftizoxime, Ceftobiprole, Ceftriaxone, Cefuroxime, Cephalosporins, Chloramphenicol, Cilastatin, Ciprofloxacin, Clarithromycin, Clindamycin, Cloxacillin, Colistin, Co-trimoxazole, Dalfopristin, Demeclocycline, Dicloxacillin, Dirithromycin, Doripenem, Doxycycline, Enoxacin, Ertapenem, Erythromycin, Ethambutol, Flucloxacillin, Fosfomycin, Furazolidone, Fusidic acid, Gatifloxacin, Geldanamycin, Gentamicin, Glycopeptides, Herbimycin, Imipenem, Isoniazid, Kanamycin, Levofloxacin, Lincomycin, Linezolid, Lomefloxacin, Loracarbef, Macrolides, Mafenide, Meropenem, Methicillin, Metronidazole, Mezlocillin, Minocycline, Monobactams, Moxifloxacin, Mupirocin, Nafcillin, Neomycin, Netilmicin, Nitrofurantoin, Norfloxacin, Ofloxacin, Oxacillin, Oxytetracycline, Paromomycin, Penicillin, Penicillins, Piperacillin, Platensimycin, Polymyxin B, Polypeptides, Prontosil, Pyrazinamide, Quinolones, Quinupristin, Rifampicin, Rifampin, Roxithromycin, Spectinomycin, Streptomycin, Sulfacetamide, Sulfamethizole, Sulfanilamide, Sulfasalazine, Sulfisoxazole, Sulfonamides, Teicoplanin, Telithromycin, Tetracycline, Tetracyclines, Ticarcillin, Tinidazole, Tobramycin, Trimethoprim, Trimethoprim-Sulfamethoxazole, Troleandomycin, Trovafloxacin, and Vancomycin. Active agents also include Aldosterone, Beclomethasone, Betamethasone, Corticosteroids, Cortisol, Cortisone acetate, Deoxycorticosterone acetate, Dexamethasone, Fludrocortisone acetate, Glucocorticoids, Hydrocortisone, Methylprednisolone, Prednisolone, Prednisone, Steroids, and Triamcinolone. Antiviral agents include abacavir, acyclovir, acyclovir, adefovir, amantadine, amprenavir, an antiretroviral fixed dose combination, an antiretroviral synergistic enhancer, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fomivirsen, fosamprenavir, foscarnet, fosfonet, fusion inhibitor, ganciclovir, gardasil, ibacitabine, idoxuridine, imiquimod, immunovir, indinavir, inosine, integrase inhibitor, interferon, interferon type I, interferon type II, interferon type III, lamivudine, lopinavir, loviride, maraviroc, MK-0518, moroxydine, nelfinavir, nevirapine, nexavir, nucleoside analogues, oseltamivir, penciclovir, peramivir, pleconaril, podophyllotoxin, protease inhibitor, reverse transcriptase inhibitor, ribavirin, rimantadine, ritonavir, saquinavir, stavudine, tenofovir, tenofovir disoproxil, tipranavir, trifluridine, Trizivir, tromantadine, truvada, valaciclovir, valganciclovir, vicriviroc, vidarabine, viramidine, zalcitabine, zanamivir, and zidovudine. Any suitable combination of these active agents is also contemplated.

### Use of GSK-3 Inhibitors to Treat Infectious Disorders

The present invention further is directed to the use of GSK-3 inhibitors which inhibit PD-1 transcription and expression and/or which promote Tbet transcription and expression in vitro or in vivo for the treatment of infectious diseases wherein suppression of PD-1 and/or increased Tbet and enhanced cellular immunity or T_{H}1 or CD4⁺ or CD8⁺ T cells or increased cytoxic T cell immunity is therapeutically desired. Examples of thereof include infectious diseases associated with bacteria, viruses, yeast or other fungi and parasites.

Examples of viral infections treatable by the present invention include those caused by single or double stranded RNA and DNA viruses, which infect animals, humans and plants, such as retroviruses, poxviruses, immunodeficiency virus (HIV) infection, echovirus infection, parvovirus infection, rubella virus infection, papillomaviruses, congenital rubella infection, Epstein-Barr virus infection, mumps, adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, and yellow fever, adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV).

More specific examples of viral infections treatable by the use of a GSK-3 inhibitor which inhibits PD-1 expression and/or promotes Tbet expression by immune cells such as T cells include, Abelson murine leukemia virus, Abelson's virus, Acute laryngotracheobronchitis virus, Adelaide River virus, Adeno associated virus group, Adenoviridae, Adenovirus, African horse sickness virus, African swine fever virus, AIDS virus, Aleutian mink disease parvovirus, Alfalfa mosaic virus, Alpharetrovirus, Alphavirus, ALV related virus, Amapari virus, Andean potato mottle virus, Aphthovirus, Aquareovirus, arbovirus, arbovirus C, arbovirus group A, arbovirus group B, Arenavirus group, Argentine hemorrhagic fever virus, Argentinian hemorrhagic fever virus, Arterivirus, Astrovirus, Ateline herpesvirus group, Aujezky's disease virus, Aura virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B19 virus, Babanki virus, baboon herpesvirus, bacterial virus, baculovirus, barley yellow dwarf virus, Barmah Forest virus, bean pod mottle virus, bean rugose mosaic virus, Bebaru virus, Beet yellows virus, Berrimah virus, betaretrovirus, Birnavirus, BK virus, Black Creek Canal virus, bluetongue virus, Bolivian hemorrhagic fever virus, Boma disease virus, border disease of sheep virus, Borgore Virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, bracovirus, broad bean mottle virus, broad bean stain virus, brome mosaic virus, Bromovirus, Buggy Creek virus, Bunyavirus, Burkitt's lymphoma virus, Bwamba fever CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, Capillovirus, caprine arthritis virus, caprine encephalitis virus, Caprine Herpes Virus, Capripox virus, Cardiovirus, Carlavirus, Carmovirus, carrot mottle virus, Cassia yellow blotch virus, Caulimovirus, Cauliflower mosaic virus, caviid herpesvirus 1, Cercopithecine herpesvirus 1, Cercopithecine herpesvirus 2, cereal yellow dwarf virus, Cetacean pox virus, Chandipura virus, Changuinola virus, channel catfish virus, Charleville virus, Chickenpox virus, Chikungunya virus, chimpanzee herpesvirus, chub reovirus, chum salmon virus, Closterovirus, Cocal virus, Coho salmon reovirus, Coital exanthema virus, Cotia virus (CPV)[, Colorado tick fever virus, Coltivirus, Columbia SK virus, Commelina yellow mottle virus, common cold virus, Comovirus, congenital cytomegalovirus, contagious, ecthyma virus, contagious pustular dermatitis virus, Coronavirus, Corriparta virus, coryza virus, cowpea chlorotic mottle virus, cowpea mosaic virus, cowpea virus, cowpox virus, coxsackie virus, CPV (cytoplasmic polyhedrosis virus,) cricket paralysis virus, Crimean-Congo hemorrhagic fever virus, croup associated virus, Crypotovirus, cucumber yellows virus, Cucumovirus, Cypovirus, cytomegalovirus, cytomegalovirus group, cytoplasmic polyhedrosis virus, deer papillomavirus, defective virus, deltaretrovirus, Dengue, Densovirus, Dependovirus, Dhori virus, Dianthovirus, diploma virus, Dolphin poxvirus (DOV)[3], DNA virus, Drosophila C virus, duck hepatitis B virus, duck hepatitis virus 1, duck hepatitis virus 2, duovirus, Duvenhage virus, Deformed wing virus (DWV), eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EMC virus, Emiliania huxleyi virus, 86 encephalitis virus, encephalomyocarditis group virus, encephalomyocarditis virus, Enterovirus, Entomopoxvirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious, anemia virus, equine morbillivirus, equine rhinopneumonitis virus, equine rhinovirus, Eubenangu virus, European elk papillomavirus, European swine fever virus, Fabavirus, fetid herpesvirus 1, feline alicivirus, feline fibrosarcoma virus, feline herpesvirus, feline immunodeficiency virus, feline infectious, peritonitis virus, feline leukemia/sarcoma virus, feline leukemia virus, feline panleukopenia virus, feline parvovirus, feline sarcoma virus, feline syncytial virus, Fijukivirus, Filovirus, Flanders virus, Flavivirus, foot and mouth disease virus, Fort Morgan virus, Four Corners hantavirus, fowl adenovirus 1, fowlpox virus, Friend virus, Furovirus, gammaretrovirus, GB virus C, Geminivirus, German measles virus, Getah virus, gibbon ape leukemia virus, green monkey virus (mullburg), glandular fever virus, goatpox virus, golden shinner virus, Gonometa virus, goose parvovirus, granulosis virus, Gray kangaroo pox virus, Gross' virus, ground squirrel hepatitis B virus, group A arbovirus, Guanarito virus, guinea pig cytomegalovirus, guinea pig type C virus, Hantavirus, hard clam reovirus, hare fibroma virus, HCMV (human cytomegalovirus), helper virus, hemadsorption virus 2, hemagglutinating virus of Japan, hemorrhagic fever virus, hendra virus, Hepadnavirus, hepatitis A virus, hepatitis B virus, hepatitis C virus, hepatitis D (delta) virus, hepatitis E virus, hepatitis F virus, hepatitis G virus, hepatitis nonA, nonB virus, hepatoencephalomyelitis reovirus 3, Hepatovirus, heron hepatitis B virus, herpes B Virus, herpes simplex virus, herpes simplex virus, 1 herpes simplex virus, herpesvirus, herpes zoster herpesvirus 7, Herpesvirus ateles Herpesvirus hominis, Herpesvirus infection, Herpesvirus saimiri, Herpesvirus suis, Herpesvirus varicellae, Highlands J virus, Hirame rhabdovirus, hog cholera virus, Hordeivirus (HODS), human adenovirus 2, human alphaherpesvirus 1, human alphaherpesvirus 2, human alphaherpesvirus 3, human B lymphotropic virus, human betaherpesvirus 5, human coronavirus, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immunodeficiency virus 1, human immunodeficiency virus 2, Human metapneumovirus hMPV, Human parainfluenza viruses, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I. human T lymphotropic virus II, human T lymphotropic virus III, ichnovirus, Ilarvirus, infantile gastroenteritis virus, infectious bovine rhinotracheitis virus, infectious haematopoietic necrosis virus, infectious pancreatic necrosis virus, infectious salmon anemia virus, influenza A virus, influenza B virus, influenza virus (unspecified), influenzavirus, (unspecified), influenzavirus A, influenzavirus B, influenzavirus C, influenzavirus D, influenzavirus pr8, iridovirus, Japanese B virus, Japanese encephalitis virus, JC virus, Junin virus, Johnson grass mosaic virus, Kaposi's sarcoma-associated herpesvirus, Kemerovo virus, Kilham's rat virus, Klamath virus, Kolongo virus, Korean hemorrhagic fever virus, kumba virus, Kunjin virus, Kyasanur forest disease, Kyzylagach virus, La Crosse virus, lactic dehydrogenase elevating virus, lactic dehydrogenase virus, Lagos bat virus, Lambda phage, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, Luteovirus, Lymphadenopathy Associated Virus, Lymphocryptovirus, lymphocytic choriomeningitis virus, lymphoproliferative virus group, Lyssavirus, Machupo virus, mad itch virus, maize chlorotic dwarf virus, maize rough dwarf virus, mammalian type B oncovirus group, mammalian type B retroviruses, mammalian type C retrovirus group, mammalian type D retroviruses, mammary tumor virus, Mapuera virus, Marafivirus, Marburg virus, Marburg-like virus, Marmosetpox virus (MPV), Mason Pfizer monkey virus, Mastadenovirus, Mayaro virus, ME virus, measles virus, Melandrium yellow fleck virus, Menangle virus, Mengo virus, Mengovirus, Merkel cell polyomavirus, Middelburg virus, milkers nodule virus, Mink enteritis virus, minute virus of mice, MLV related virus, MM virus, Mokola virus, Molluscipoxvirus, Molluscum contagiosum virus, Molluscum-like pox virus (MOV), monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, Mule deerpox virus (DPV, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Necrovirus, Neethling virus, Nelson Bay virus, Nemtick Virus, Neopvirus, neurotropic virus, New World Arenavirus, newborn pneumonitis virus, Newcastle disease virus, Nipah virus, noncytopathogenic virus, Norovirus, Norwalk virus, nuclear polyhedrosis virus (NPV), nipple neck virus, O'nyong'nyong virus, oat sterile dwarf virus, Ockelbo virus, oncogenic virus, oncogenic virus like particle, oncornavirus, Orbivirus, Orf virus, Oropouche virus, Orthohepadnavirus, orthomyxovirus, Orthopoxvirus, Orthoreovirus, Orungo ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, parsnip yellow fleck virus, Parvovirus, Parvovirus B19, parvovirus group, pea enation mosaic virus, Pestivirus, Phlebovirus, phocine distemper virus, Phytoreovirus, Picodnavirus, Picornavirus, pig cytomegalovirus, pigeonpox virus, Piry virus, Pixuna virus, plant rhabdovirus group, plant virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus, papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, Potato leaf roll virus, Potato mop top virus, Potato virus Y, Potexvirus, Potyvirus, pox virus, poxvirus, poxvirus variolae, Prospect Hill virus, provirus, pseudocowpox virus, pseudorabies virus, psittacinepox virus, Puumala virus, Qalyub virus, Quail pea mosaic virus, quailpox virus, Queensland fruitfly virus, Quokkapox virus (QPV), rabbit fibroma virus, rabbit kidney vaculolating virus, rabbit papillomavirus, rabies virus, raccoon parvovirus, raccoonpox virus, radish mosaic virus, Ranikhet virus, rat cytomegalovirus, rat parvovirus, rat virus, Rauscher's virus, recombinant vaccinia virus, recombinant virus, Red Clover Necrotic Mosaic Virus, Red kangaroo poxvirus [1][8], reovirus, reovirus 1, reovirus 2, reovirus 3, reptilian type C virus, respiratory infection virus, respiratory syncytial virus, respiratory virus, reticuloendotheliosis virus, Retrovirus, Rhabdovirus, Rhabdovirus carpia, Rhadinovirus, rhinovirus, Rhizidiovirus, Rift Valley fever virus, Riley's virus, rinderpest virus, RNA tumor virus, RNA virus, Ross River virus, Rotavirus, rougeole virus, Rous, sarcoma virus, rubella virus, rubeola virus, Rubivirus, Russian autumn encephalitis virus, S6-14-03 virus, SA 11 simian virus, SA 15, SA2 virus, SA6 virus, SA8 virus, Sabia virus, Sabio virus, Sabo virus, Saboya virus, Sabulodes caberata GV, Sacbrood virus, Saccharomyces cerevisiae virus LA, Saccharomyces cerevisiae virus LBC Sagiyama virus, Saguaro cactus, virus, Saimiriine herpesvirus 1, Saimiriine herpesvirus 2, Sainpaulia leaf necrosis virus, SaintAbb's Head virus, Saint-Floris virus, Sakhalin virus, Sal Vieja virus, Salanga virus, Salangapox virus, Salehabad virus, salivary gland virus, Salmonid herpesvirus 1, Salmonid herpesvirus 2, Salmonis virus, Sambucus, vein clearing virus, Samia cynthia NPV, Samia pryeri NPV, Samia ricini NPV, Sammons' Opuntia virus, SanAngelo virus, San Juan virus, San Miguel sealion virus, San Perlita virus, Sand rat nuclear inclusion agents, Sandfly fever Naples virus, Sand fly fever Sicilian virus, Sandjimba virus, Sango virus, Santa Rosa virus, Santarem virus, Santosai temperate virus, Sapphire II virus, Saraca virus, Sarracenia purpurea virus, SARS virus, satellite virus, Sathuperi virus, Satsuma dwarf virus, Saturnia pavonia virus, Saturnia pyri NPV, Saumarez Reef virus, Sawgrass virus, Sceliodes cordalis NPV, Schefflera ringspot virus, Sciaphila duplex GV, Scirpophaga incertulas NPV, Sciurid herpesvirus, Sciurid herpesvirus 2, Scoliopteryx libatFix NPV, Scopelodes contracta NPV, Scopelodes venosa NPV, Scopula subpunctaria NPV, Scotogramma trifolii GV, Scotogramma trifolu NPV, Scrophularia mottle virus, SDAV (sialodacryoadenitis virus), sealpox virus, Selenephera lunigera NPV, Selepa celtis GV, Seletar virus, Selidosema suavis NPV, Semidonta biloba NPV, Semiothisa sexmaculata GV, Semliki Forest Virus, Sena Madureira virus, Sendai virus, Seoul virus, Sepik virus, Serra do Navio virus, Serrano golden mosaic virus, Sesame yellow mosaic virus, Sesamia calamistis NPV, Sesamia cretica GV, Sesamia inferens NPV, Sesamia nonagrioides GV, Setora nitens virus, Shallot latent virus, Shamonda virus, Shark River virus, Sheep associated malignant catarrhal fever, Sheep papillomavirus, Sheep pulmonary adenomatosis associated herpesvirus, sheeppox virus, Shiant Islands virus, Shokwe virus, Shope fibroma virus, Shope papilloma virus, Shuni virus, Siamese cobra herpesvirus, Sibine fusca adensovirus, Sida golden mosaic virus (SiGMV), Sida golden yellow vein virus (SiGYVV), Sigma virus, Sikte water-borne virus, Silverwater virus, Simbu virus, Simian adenoviruses 1 to 27, Simian agent virus, Simian enterovirus 1 to 18, simian foamy virus, Simian hemorrhagic fever virus, simian hepatitis A virus, simian human immunodeficiency virus, simian immunodeficiency virus, simian parainfluenza virus, Simian rotavirus SA11, Simian sarcoma virus, simian T cell lymphotrophic virus, Simian type D virus 1, Simian varicella herpesvirus, simian virus, simian virus, Simplexvirus, Simulium vittatum densovirus, Sin Nombre virus, Sindbis virus, Sint1em's onion latent virus, Sixgun city virus, Skunkpox virus, smallpox virus, Smelt reovirus, Smerinthus, ocellata NPV, Smithiantha virus, Snakehead rhabdovirus, Snowshoe hare virus, Snyder-Theilen feline sarcoma virus, Sobemovirus, Sofyn virus, Soil-borne wheat mosaic virus, Sokoluk virus, Soldado virus, Somerville virus 4, Sonchus mottle virus, Sonchus virus, Sonchus yellow net virus, Sorghum chlorotic spot virus, Sorghum mosaic virus, Sorghum virus, Sororoca virus, Soursop yellow blotch virus, South African passiflora virus, South American hemorrhagic fever viruses, South African passiflora virus, South River virus, Southern bean mosaic virus, Southern potato latent virus, Sowbane mosaic virus, Sowthistle yellow vein virus, Soybean chlorotic mottle virus, Soybean wrinkle leaf virus, Soybean dwarf virus, Soybean mosaic virus, SPAr-2317 virus, Sparganothis pettitana NPV, sparrowpox virus, Spartina mottle virus, Spectacled caimanpox virus, SPH 114202 virus, Sphenicid herpesvirus 1, Sphinx ligustri NPV, Spider monkey herpesvirus, Spilarctia subcarnea NPV, Spilonota ocellana NPV, Spilosoma lubricipeda NPV, Spinach latent virus, Spinach temperate virus, Spiroplasma phage 1, Spiroplasma phage 4, Spiroplasma phage aa, Spiroplasma phage C1 ITS2, Spodoptera exempta cypovirus 11, Spodoptera exempta cypovirus 12, Spodoptera exemptacypovirus 3, Spodoptera exempta cypovirus 5, Spodoptera exempta cypovirus 8, Spodoptera exempta NPV, Spodoptera exigua cypovirus 11, Spodoptera exigua GV, Spodoptera exigua MNPV, Spodoptera exigua NPV, Spodoptera frugiperda GV, Spodoptera frugiperda MNPV, Spodoptera frugiperda NPV, Spodoptera latifascia NPV, Spodoptera littoralis, Spodoptera littoralis NPV, Spodoptera litura GV, Spodoptera litura NPV, Spodoptera mauritia NPV, Spodoptera ornithogalli NPV, Spondweni virus, spring beauty latent virus, Spring viremia of carp virus, Spumavirus, Squash leaf curl virus, squash mosaic virus, squirrel fibroma virus, Squirrel monkey herpesvirus, squirrel monkey retrovirus, SR-11 virus, Sri Lankan passionfruit mottle virus, Sripur virus, SSV 1 virus group, StAbbs Head virus, St. Louis encephalitis virus, Staphylococcus, phage 107, Staphylococcus phage 187, Staphylococcus phage 2848A, Staphylococcus phage 3A, Staphylococcus phage 44A HJD, Staphylococcus phage 77, Staphylococcus phage B11-M15, Staphylococcus phage Twort, Starlingpox virus, Statice virus Y, P, STLV (simian T lymphotropic virus) type I, STLV (simian T lymphotropic virus) type II, STLV (simian T lymphotropic virus) type III, stomatitis papulosa virus, Strafford virus, Strawberry crinkle virus, Strawberry latent ringspot virus, Strawberry latent ringspot virus satellite, Strawberry mild yellow edge virus, Strawberry mild yellow edge-associated virus, Strawberry pseudo mild yellow edge virus, Strawberry vein banding virus, Streptococcus phage 182, Streptococcus, phage 2BV Streptococcus phage A25, Streptococcus phage 24, Streptococcus phage PEI, Streptococcus phage VD13, Streptococcus phage fD8, Streptococcus phage CP-1, Streptococcus phage Cvir, Streptococcus phage H39, Strigid herpesvirus 1, Striped bass reovirus, Striped Jack nervous necrosis virus, Stump-tailed macaque virus, submaxillary virus, Subterranean clover mottle virus, Subterranean clover mottle virus satellite, Subterranean clover red leaf virus, Subterranean clover stunt virus, Sugarcane bacilliform virus, Sugarcane mild mosaic virus, Sugarcane mosaic virus, Sugarcane streak virus, suid alphaherpesvirus 1, suid herpesvirus 2, Suipoxvirus, Sulfolobus virus 1, Sunday Canyon virus, Sunflower crinkle virus, Sunflower mosaic virus, Sunflower rugose mosaic virus, Sunflower yellow blotch virus, Sunflower yellow ringspot virus, Sun-hemp mosaic virus, swamp fever virus, Sweetwater Branch virus, Swine cytomegalovirus, Swine infertility and respiratory syndrome virus, swinepox virus, Swiss mouse leukemia virus, Synaxis jubararia NPV, Synaxis pallulata NPV, Synetaeris tenuifemur virus, Syngrapha selecta NPV, T4 phage, T7 phage, TAC virus, Tacaiuma virus, Tacaribe complex virus, Tacaribe virus, Taggert virus, Tahyna virus, Tai virus, Taiassui virus, Tamana bat virus, Tamarillo mosaic virus, Tamdy virus, Tamiami virus, Tanapox virus, Tanga virus, Tanjong Rabok virus, Taro bacilliform virus, Badnavirus, Tataguine virus, Taterapox virus, Taterapox virus, Poxviridae Teasel mosaic virus, Tehran virus, Telfairia mosaic virus, Telok Forest virus, Tembe virus, Tembusu virus, Tench reovirus, Tensaw virus, Tenvivirus, Tephrosia symptomless virus, Termeil virus, Tete virus, Tetralopha scortealis NPV, Tetropium cinnamoptemm NPV, Texas pepper virus, Thailand virus, Thaumetopoea pityocampa GV, Thaumetopoea pityocampa NPV, Thaumetopoea processionea NPV, Theiler's encephalomyelitis virus, Theiler's virus, Theophila mandarina NPV, Theretra japonica NPV, Thermoproteus virus 1, Thermoproteus virus 2, Thermoproteus virus 3, Thermoproteus virus 4, Thiafora virus, Thimiri virus, Thistle mottle virus, Thogoto virus, Thormodseyjarklettur virus, Thosea asigna virus, Thosea baibarana NPV, Thosea sinensis GV, Thottapalayam virus, Thylidolpteryx ephemeraeformis NPV, Thymelicus, lineola NPV, Tibrogargan virus, Ticera castanea NPV, Tick borne encephalitis virus, Tillamook virus, Tilligerry virus, Timbo virus, Tilmboteua virus, Tilmaroo virus, Tindholmur virus, Tinea pellionella NPV, Tineola hisselliella NPV, Tinpula paludosa NPV, Tinracola plagiata NPV, Tioman virus, Titi monkey adenovirus, Tlacotalpan virus, Tobacco bushy top virus, Tobacco etch virus, Tobacco leaf curl virus, Tobacco mild green mosaic virus, tobacco mosaic virus, Tobacco mosaic virus satellite, Tobacco mottle virus, Tobacco necrosis virus, Tobacco necrosis virus satellite, Tobacco necrosis virus small satellite, Tobacco necrotic dwarf virus, tobacco rattle virus, Tobacco ringspot virus, Tobacco ringspot virus satellite, Tobacco streak virus, Tobacco stunt virus, Tobacco vein banding mosaic virus, Tobacco vein distorting virus, Tobacco vein mottling virus, Tobacco wilt virus, Tobacco yellow dwarf virus, Tobacco yellow net virus, Tobacco yellow vein assistor virus, Tobacco yellow vein virus, Tobamovirus, Tobravirus, Togavirus, Tomato apical stunt viroid, Tomato aspermy virus, Tomato black ring virus, Tomato black ring virus satellite, Tomato bunchy top viroid, tomato bushy stunt virus, Tomato bushy stunt virus satellite, Tomato golden mosaic virus, Tomato leaf crumple virus, Tomato leaf curl virus-Au, Tomato leaf curl virus-In, Tomato leafroll virus, Tomato mosaic virus, Tomato mottle virus, Tomato pale chlorosis virus, Tomato planta macho viroid, Tomato pseudo-curly top virus, Tomato ringspot virus, Tomato spotted wilt virus, Tomato top necrosis virus, Tomato vein yellowing virus, Tomato yellow dwarf virus, Tomato yellow leaf curl virus-Is, Tomato yellow leaf curl virus-Sr, Tomato yellow leaf curl virus-Th, Tomato yellow leaf curl virus-Ye, Tomato yellow mosaic virus, Tomato yellow top virus, Tombus virus, Tongan vanilla virus, Tony Virus, Torovirus, Tortrix loeflingiana NPV, Tortrix viridana NPV, Toscana virus, Tospovirus, Toxorhynchites brevipalpis NPV, Trabala vishnou NPV, Tradescantia/Zebrina virus, Trager duck spleen necrosis virus, Tranosema sp. virus, Transforming virus, Tree shrew adenovirus 1, Tree shrew herpesvims, Triatoma virus, Tribec virus, Trichiocampus irregularis NPV, Trichiocampus viminalis NPV, Trichomonas vaginalis virus, Trichoplusia ni cypovirus, Trichoplusia ni granulovirus, Trichoplusia ni MNPV, Trichoplusia ni Single SNPV, Trichoplusia ni virus, Trichosanthes mottle virus, Triticum aestivum chlorotic spot virus, Trivittatus virus, Trombetas virus, Tropaeolum virus 1, Tropaeolum virus 2, Trubanarnan virus, Tsuruse virus, Transfusion Transmitted Virus (TT Virus), TTV-like minivirus (TLMV), Tucunduba virus, Tulare apple mosaic virus, Tulip band breaking virus, Tulip breaking virus, Tulip chlorotic blotch virus, Tulip top breaking virus, Tulip virus X, tumor virus, Tupaia virus, Tupaiid herpesvirus 1, Turbot herpesvirus, Turbot reovirus, Turkey adenoviruses 1 to 3, Turkey coronavirus, Turkey herpesvirus 1, Turkey rhinotracheitis virus, Turkeypox virus, Turlock virus, Tymovirus, Tyuleniy virus, type C retroviruses, type D oncovirus, type D retrovirus group, Uasin Gishu disease virus, Uganda S virus, Ugymyia sericariae NPV, ulcerative disease rhabdovirus, Ullucus mild mottle virus, Ullucus mosaic virus, Ullucus virus C, Umatilla virus, Umbre virus, Una virus, Upolu virus, UR2 sarcoma virus, Uranotaenia sapphirina NPV, Urbanus proteus NPV, Urucuri virus, Ustilago maydis virus 1, Ustilago maydis virus 4, Ustilago maydis virus 6, Usutu virus, Utinga virus, Utive virus, Uukuniemi virus group, Vaccinia virus, Vaeroy virus, Vallota mosaic virus, Vanessa atalanta NPV, Vanessa cardui NPV, Vanessa prorsa NPV, Vanilla mosaic virus, Vanilla necrosis virus, Varicella zoster virus, Varicellovirus, Varicola virus, Variola major virus, Variola virus, Vasin Gishu disease virus, Vellore virus, Velvet tobacco mottle virus, Velvet tobacco mottle virus satellite, Venezuelan equine encephalitis virus, Venezuelan Equine Encephalomyelitis virus, Venezuelan hemorrhagic fever virus, Vesicular stomatitis Alagoas virus, Vesicular stomatitis Indiana virus, Vesicular stomatitis New Jersey virus, Vesiculovirus, Vibrio phage 06N-22P, Vibrio phage 06N-58P, Vibrio phage 4996, Vibrio phage a3a, Vibrio phage I, Vibrio phage II, Vibrio phage m, Vibrio phage IV, Vibrio phage kappa, Vibrio phage nt-1, Vibrio phage OXN-52P, Vibrio phage OXN-IOOP, Vibrio phage v6, Vibrio phage Vfl2, Vibrio phage Vf33, Vibrio phage VP1, Vibrio phage VP11, Vibrio phage VP3, Vibrio phage VP5, Vibrio phage X29, Vicia cryptic virus, Vigna sinensis mosaic virus, Vilyuisk virus, Vinces virus, Viola mottle virus, viper retrovirus, viral haemorrhagic septicemia virus, virus-like particle, Visna Maedi virus, Visna virus, Voandzeia mosaic virus, Voandzeia necrotic mosaic virus, volepox virus, Wad Medani virus, Wallal virus, Walleye epidermal hyperplasia, Walrus calicivirus, Wanowrie virus, Warrego virus, Watermelon chlorotic stunt virus, Watermelon curly mottle virus, Watermelon mosaic virus 1, Watermelon mosaic virus 2, Weddel water-borne virus, Weldona virus, Wesselsbron virus, West Nile virus, Western equine encephalitis virus, Western equine encephalomyelitis virus, Wexford virus, Whataroa virus, Wheat American striate mosaic virus, Wheat chlorotic streak virus, Wheat dwarf virus, Wheat rosette stunt virus, Wheat spindle streak mosaic virus, Wheat streak mosaic virus, Wheat yellow leaf virus, Wheat yellow mosaic virus, White bryony mosaic virus, White bryony virus, White clover cryptic virus 1, White clover cryptic virus 2, White clover cryptic virus 3, White clover mosaic virus, White lupinmosaic virus, Wild cucumber mosaic virus, Wild potato mosaic virus, Wildbeest herpesvirus, Wineberry latent virus, Winter wheat mosaic virus, Winter wheat Russian mosaic virus, Wiseana cervinata GV Wiseana cervinata NPV, Wiseana signata NPV, Wiseana umbraculata GV, Wiseana umbraculata NPV, Wissadula mosaic virus, Wisteria vein mosaic virus, Witwatersrand virus, Wongal virus, Wongorr virus, Winter Vomiting Virus, Woodchuck hepatitis B virus, Woodchuck herpesvirus marmota 1, Woolly monkey sarcoma virus, Wound tumor virus, WRSV virus, WVU virus 2937, WW virus 71 to 212, Wyeomyia smithii NPV, Wyeomyia virus, Xanthomonas phage Cf, Xanthomonas phage Cflt, Xanthomonas phage RR66, Xanthomonas phage Xf, Xanthomonas phage Xf2, Xanthomonas phage XP5, Xenopus virus T21, Xiburema virus, Xingu virus, Xylena curvimacula NPV, Y73 sarcoma virus, Yaba monkey tumor virus, Yaba-1 virus, Yaba-7 virus, Yacaaba virus, Yam mosaic virus, Yaounde virus, Yaquina Head virus, Yatapoxvirus, Yellow fever virus, Yogue virus, Yokapox virus, Yokase virus, Yponomeuta cognatella NPV, Yponomeuta evonymella NPV, Yponomeuta malinellus NPV, Yponomeuta padella NPV, Yucca baciliform virus, Yug Bogdanovac virus, Zaliv Terpeniya virus, Zea mays virus, Zegla virus, Zeiraphera diniana GV, Zeiraphera diniana NPV, Zeiraphera pseudotsugana NPV, Zika virus, Zirqa virus, Zoysia mosaic virus, Zucchini yellow fleck virus, Zucchini yellow mosaic virus or Zygocactus virus.

It is especially contemplated to treat “Human Immunodeficiency Virus” or “HIV” infection which refer to the disease caused by the HIV virus which results in the failure of the host immune system and development of Acquired Immunodeficiency Syndrome (AIDS). With respect thereto, HIV infection has been linked to PD-1 down-regulation in, for example, Barber et al., Nature 439, 682 (2006) and Day et al., Nature 443, 350 (2006). Without being bound by theory, the data presented herein therefore provides the novel use of GSK-3 inhibitors to treat HIV-1 infection by inhibiting or arresting PD-1 expression or promoting Tbet expression.

It is further especially contemplated to treat “Lymphocytic Choriomeningitis” or “LCM” which refers to the viral infection caused by Lymphocytic Choriomeningitis Virus (LCMV) which results in inflammation of the membranes surrounding the brain and spinal cord and of the cerebrospinal fluid. LCM has been linked to Tbet modulation in, for example, Sullivan et al., Proc. Natl. Acad. Sci. 100, 15818 (2003). Without being bound by theory, the data presented herein therefore provides the novel use of GSK-3 inhibitors to treat LCMV by blocking PD-1 expression or promoting Tbet expression.

It is also especially contemplated to treat “Herpes Simplex Virus type 2” or “HSV-2” infection with GSK-3 inhibitors according to the invention which refers to the viral infection caused by Herpes Simplex Virus (HSV) which results in blisters and cold sores forming on the skin or mucous membranes of the body, in particular, in and around the mouth, lips or genitals. HSV can evade the immune system and lie dormant in a host causing chronic, persistent infection. HSV-2 infection has been linked to Tbet modulation in, for example, Svensson et al., J. Immunol. 174, 6266 (2005). Without being bound by theory, the data presented herein suggests that GSK-3 inhibitors which promote Tbet expression or reduce PD-1 expression by T cells may be used to treat Herpes.

It is also especially contemplated to treat “Hepatitis B” infection with GSK-3 inhibitors according to the invention or “Hepatitis C” infection. These viral infections usually result in inflammation of the liver and can lead to cirrhosis or liver cancer if the infection becomes chronic.

The subject therapies may comprise the use of at least one GSK-3 inhibitor that reduces PD-1 expression and/or increase Tbet expression by immune cells (T cells) as a monotherapy but more typically will be part of a therapeutic regimen that includes the administration of other antiviral drugs such as small molecules, antibodies, antisense RNA, RNAi's, antibodies or other immune modulatory agents.

Examples of antiviral agents include nucleoside or nucleotide analogs, protease inhibitors, or other antiviral agents including the following Abacavir, “Ziagen” or “Trizivir” or “Kivexa/Epzicom”, Aciclovir—anti-HSV, Acyclovir, Adefovir, Amantadine, Amprenavir, Ampligen, Arbidol, Atazanavir, Atripla, Balavir, Boceprevirertet, Cidofovir, Combivir, Dolutegravir, Darunavir, Delavirdine, Didanosine, Docosanol, Edoxudine, Efavirenz, Emtricitabine, Enfuvirtide, Entecavir, Entry inhibitors, Famciclovir, Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitors, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon α-2a, Penciclovir, Peramivir, Pleconaril, Podophyllotoxin, Protease inhibitors, Raltegravir, Reverse transcriptase inhibitors, Ribavirin, Rimantadine, Ritonavir, Pyramidine, Saquinavir, Sofosbuvir, Stavudine, Tea tree oil, Telaprevir, Tenofovir, Tenofovir disoproxil, Tipranavir, Trifluridine, Trizivir, Tromantadine, Truvada, traporved, Valaciclovir (Valtrex), Valganciclovir, Vicriviroc, Vidarabine, Viramidine, Zalcitabine, Zanamivir (Relenza), Zidovudine and combinations of any of the foregoing including synergistic combinations.

In a further embodiment, the infectious disease treated may comprise a parasitic or bacterial infection. In another embodiment, the disease is an infection where the infection results in a musculature disease, or an ear disease, or an eye disease, or a nervous disorder or a skin disease or cardiovascular disease or endocrine disorder or a gastro-intestinal or enteric disease. In one embodiment, the disease is an infection that causes a kidney disease or an autoimmune or inflammatory disease or an autoimmune disease of blood disease, musculature, ear, eye disease, kidney, or skin. In a further embodiment, the disease is an infection where the infection is a systemic autoimmune disease. In a further embodiment, the autoimmune disease is pernicious anemia, autoimmune hemolytic anemia, aplastic anemia, idiopathic thrombocytopenic purpura, ankylosing spondylitis, polymyositis, dermatomyositis, autoimmune hearing loss, Meniere's syndrome, Mooren's disease, Reiter's syndrome, Vogt-Koyanagi-Harada disease, glomerulonephritis, IgA nephropathy; diabetes mellitus (type I), pemphigus, pemphigus vulgaris, pemphigus foliaceus, pemphigus erythematosus, bullous pemphigoid, vitiligo, epidermolysis bullosa acquisita, alopecia areata; autoimmune myocarditis, vasculitis, Churg-Strauss syndrome, giant cells arteritis, Kawasaki's disease, polyarteritis nodosa, Takayasu's arteritis and Wegener's granulomatosis, Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-I) polyglandular autoimmune syndrome type 2 (PAS-2), and polyglandular autoimmune syndrome type 3 (PAS-3), including autoimmune hepatitis, primary biliary cirrhosis, inflammatory bowel disease, celiac disease, Crohn's disease, including multiple sclerosis, myasthenia gravis, Guillan-Barre syndrome and chronic inflammatory demyelinating neuropathy, including systemic lupus erythematosus, antiphospholid syndrome, autoimmune lymphoproliferative disease, autoimmune polyendocrinopathy, Behçet's disease, Goodpasture's disease, rheumatoid arthritis, osteoarthritis, septic arthritis, sarcoidosis, scleroderma and/or Sjögren's syndrome.

The invention especially contemplates the treatment of inflammatory bowel disease” or “IBD” refers to a group of inflammatory conditions of the colon and small intestine. IBD has been linked to PD-1 modulation in, for example, Neurath et al., J. Exp. Med. 195, 1129 (2002), which describes PD-1−/− mice to be more susceptible to Th2-mediated colitis than control littermates. Without being bound by theory, the data presented herein therefore provides the novel use of GSK-3 inhibitors to treat IBD by promoting T_{H}1 immunity.

Indirect evidence requires re-creation of the human disease in an animal model. The majority of autoimmune diseases fit in this category. For example, gene knock-out mice have provided the best models of inflammatory bowel disease; neonatal thymectomy of mice can produce excellent models of infectious vulnerability. At the same time, animal models must be viewed with caution because they invariably differ to some degree from the human disease. “Crohn's disease” is a chronic inflammatory disorder, in which the body's own immune system attacks the gastrointestinal tract causing discomfort, pain and inflammation. Crohn's disease has been linked to PD-1 modulation in, for example, Neurath et al., J. Exp. Med. 195, 1129 (2002). Without being bound by theory, the data presented herein therefore provides the novel use of GSK-3 inhibitors to treat Crohn's disease.

Bacterial diseases treatable by the invention include by way of example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella.

Other specific examples of bacterial infections treatable according to the invention include, but are not limited to, Bordetella pertussis (which may cause Pertussis), Borrelia burgdorferi, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani (which may cause Tetanus), Corynebacterium diphtheriae (which may cause Diphtheria), Echinococcus (which may cause Echinococcal disease), Enterococcus faecalis, Enterococcus faecium, Escherichia coli (which may cause diarrhea, hemolytic uremic syndrome or urinary tract infection) such as Enterotoxigenic E. coli, Enteropathogenic E. coli, Enterohemorrhagic E. coli or Enteroaggregative E. coli, Francisella tularensis, Haemophilus influenzae (which may cause respiratory infections or meningitis), Helicobacter pylori (which may cause gastritis, peptic ulcer disease or gastric neoplasms), Legionella pneumophila, Leptospira interrogans, Listeria monocytogenes, Mycobacterium leprae, Mycobacterium tuberculosis (which may cause tuberculosis), Mycobacterium ulcerans, Mycoplasma pneumonia, Neisseria gonorrhoeae, Neisseria meningitides, Pneumococcus (which may cause meningitis, pneumonia, bacteremia or otitis media), Pseudomonas aeruginosa, Rickettsia rickettsia, Salmonella (which may cause food poisoning) such as, Salmonella bongo, Salmonella enterica, Salmonella subterranean, Salmonella typhi or Salmonella typhimurium, Shigella (which may cause shigellosis or gastroenteritis) such as Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Vibrio cholerae (which may cause cholera) or Yersinia pestis.

With further respect to this therapeutic application of the invention, bacterial infections have been linked to PD-1 and PD-1 modulation in, for example, Ravindran et al., J. Immunol. 175, 4603 (2005) and Sullivan et al., J. Immunol. 175, 4593 (2005), Hu et al., Mol. Med. Report 6, 139 (2012) and Szabo et al., Science 295, 338 (2002). Without being bound by theory, the data presented herein provides the novel use of GSK-3 inhibitors to treat bacterial infections, such as Salmonella and Mycobacterium tuberculosis by promoting CTL or T_{H}1 immunity.

Other preferred examples of bacterial infections treatable with GSK-3 inhibitors according to the invention may include “Salmonella” infections caused by the gram-negative bacteria of the Salmonella family. Infections are usually the result of food poisoning and serious symptoms can develop, especially in those with a weak or suppressed immune system; and “Mycobacterium tuberculosis” infections, which is the most common cause of tuberculosis (causes chronic infection of the lungs and is difficult to treat due to the length of treatment and the development of drug-resistant strains).

The invention contemplates bacterial infection treatment regimens which administer at least one GSK-3 inhibitor which inhibits PD-1 expression and or promotes Tbet expression by T cells, which is administered alone or as part of a therapeutic regimen that includes the use of other active agents such as antibiotics or other immune modulars.

Examples of antibiotics that may be administered in association with a GSK-3 inhibitor according to the invention include Actinomycin D, Actinosin, Aculeacin A, Acycloguanosine, Adenine 9-β-D-arabinofuranoside, Alamethicin, L-Alanyl-L-1-aminoethylphosphonic acid Albendazole, 17-(Allylamino)-17-demethoxygeldanamycin, Amastatin hydrochloride hydrate, Amikacin disulfate salt, Amikacin hydrate aminoglycoside, Amikacin sulfate salt, 7-Aminoactinomycin D, 7-Aminoactinomycin D, 7-Aminocephalosporanic acid, 7-Aminodesacetoxycephalosporanic acid, N-(6-Aminohexyl)-5-chloro-1-naphthalenesulfonamide hydrochloride, amoxicillin, amphotericin B, anisomycin, anhydretythtromycin, antimycin, apicidin, apoptolipina, apramycin sulfate, artesunate, asochlorin, ascomycin, 5-axzacytidine, azaserine, azithromycin, azlocillin, bacitracin, bafilomycin, bestatin, beta d-4, bithionol, blasticidine, bleomycin, borrelidin, brefeldin, caerulomycin, calcium ionophore iii selectophore, calcium ionophore A23187, camptothecin, capreomycin, carbadox, carbenicillin, carboplatin, cecropin, cefaclor, ceflexin VETRANAL, cephalexin, cefixime, cefmetazole, cefoperazone, cefotaxime, cefsulodin, ceftazidime, ceftriaxone, cephalexin, cephalomaine, cephalothin, cephradine, cerosporin, cerulenin, cetylpyridinium, chloramphenicol, chlorhexidine, chloroquine, chlortetracycline, chromomycin, chrysomysin, cinnamysin, cinoxacin, ciprofloxacin, clarithromycin, clebopride maleate, clindamycin, clofazimine, clotrimazole, cloxacillin, colistatin sulfate, concanamycin a, cordycepin, coumermycin, cryptotanshinone, cycloheximide, cycloserine, cyclosporin, cycichalasin, dacarbazine, daunorubicin, decoyine, defensin, demeclocycline, 1-deoxymannojirimycin, dermaseptin, dichlorophene, dicloxacillin, diethylcarbamazine, difloxacin, dihydrostreptomycin sesquisulfate, diloxanide, dimetridazole, diminazene, dirithromycin, doxorubicin, doxycycline, econazole, elfin, embelin, emetine. Erofloxacin, erythromycin, ethambutol. Filipin, florfenicol, flubendazole, fluconazole, flumequine, flumethasone, 5-fluorocytiosime nucleoside analog, flurbiprofen, fumagillin, fumitremorgin c, furazolidone, fusaric acid, fusidic acid, G418, ganciclovir, geldanamycin, gentamicin, gliotoxin, I-glutamine penicillin, gramicidin, gramicidin a, gramicidin c, griseofulvin, herbimycin, hexadecylpyridinium chloride monohydrate, honokiol, hydrocortisone acetate, 8-hydroxyquinoline, 4-hydroxytamoxoifen, hygromycin b, ikarugumycin, imipenem, indomethacin, ionomycin, Irgasan, itraconazole, iturin a, ivermectin, josamycin, k-252a, k252b, kanamycin, kasugamycin, kandomycin, ketoconazole, kirronmycin, lactic acid, lactoferricin, leptomycin, levamisole, levofloxacin, lincomycin, 11-37, lomefloxacin, lysostaphin, magainin mebendazole, meclocycline, menadione, 2-mercaptopyridine n-oxide salt, n-methyl-1-deoxynojirimycin, metronidazole, miconazole, minocycline, mithramycin a, mitomycin c, monensin salt, morantel salt, moxalactam, mupirocin, mycophenolic acid, nafcillin salt, naftifine hydrochloride, nalidixic acid, narasin, neocarzinostatin, neomycin, netilmicin, nestropsin dihydrochloride, nicarbazin, niclosamide, nigericin, nikkomycin z, nisin, nitrofurantoin, nonactin, norfloxacin, novobiocin, nystatin, ochratoxin A, ofloxacin fluoroquinolone, oligomycin, oligomycin a or b, oxacillin, oxantel, oxolinic acid, oxytetracycline dihydrate, oxytetracycline hemicalcium salt or dihydrochloride, paclitaxel, paromomycin, patulin, pd 404, pediocoin, pefloxacin, d-penicillamine, penicillin g, penicillin-streptomycin, pentamidine isethionate, phenazine methosulfate, phenoxymethylpenicillinic acid potassium salt, peliomycin, phosphomycin, pimaricin, pipemidic acid, piperacillin, pirarubicin, polymyxin b, potassium clavulanate, praziquantel anethelmic, praziquantel, puromycin, pyrantel, pyrazinecarboxamide, pyronaridine tetraphosphate, pyrrolnitrin, quinine hemisulfate salt, quinine sulfate, 8-quinolinol, radicicola, ramoplanin, rapamycin, rebeccamycin, reveromycin A, ribavirin, ribostamycin sulfate salt, ricobendazole, rifabutin, rifampicin, rifapentine, rifaximin, ristomycin monosulfate, rolitetracycline, roxithromycin, salinomycin, sangivamycin, sinefungin, sisomicin, sorbic acid, sordarin sodium salt, sparfloxacin, spectinomycin, spergualin trihydrohydrochloride, spiramycin, spiramycin adipate, staurosporine, streptolysin D, streptolysin O, streptomycin, streptomycin sulfate, streptonigrin, streptozocin, succinylsulfathiazole, sulconazole nitrate salt, sulfabenzamide, sulfacetamide, suifachloropyridazine, sulfadiazine, sulfadimethoxine, sulfadimidine, sulfadoxine. Sulfaguanidine, sulfameter, sulfamethazine, sulfamonomethoxine, sulfanilamide, sulfanitran, sulfasalazine, sulfathiazole sodium salit, sulochrin, surfactin, swainsonine, syringomycin E, tamoxifen, tazobactam, teicoplanin, terbinafine hydrochloride, tetracycline, tetramisole HCl, thiabendazole, thiamphenicol, thimerosal, thioplutin, thiostrepton, thio-tepa, thymol, tiamulin, ticarcillin, tioconazole, tobramycin, tolnasulfate, triacsin C, trichlorfon pestanal, trimethoprim, tubercidin, tunicamycin, tunicamycin C2, tylosin, valacyclovir, valinomycin, vancomycin HCl, vinblastine sulfate, vincristine, virginiamycin S1, virginiamycin M1 and salts and derivatives or combinations of any of the foregoing.

Also, the present invention contemplates the use of GSK-3 inhibitors which inhibit PD-1 transcription and expression and/or promote Tbet expression to treat fungal and yeast infections or mycoses, e.g., superficial mycoses resulting from Tinea versicolor, cutaneous mycoses such as are caused by Microsporum, Trichophyton, and Epidermophyton fungi, and systemic mycoses caused by fungi such as chlamydia, candidiasis, aspergillosis, histoplasmosis, and cryptococcal meningitis.

The invention contemplates treatment of fungal or yeast treatment comprising the use of at least one GSK-3 inhibitor which inhibits PD-1 expression and/or increases Tbet expression as a monotherapy or in conjunction with other actives such as anti-fungal agents such as fluconazole, or Diflucan, amphotericin B, Tolnaftate (Tinactin), Ketoconazole, Itraconazole; Terbinafine (Lamisil); Echinocandins (caspofungin); Griseofulvin, tioconazole and others generally known in the art.

Further, the present invention contemplates the use of GSK-3 inhibitors which inhibit PD-1 transcription and expression and/or promote Tbet expression to treat parasitic diseases including but not limited to those caused by plasmodium (malaria), Amoebiasis, Enterobiasis, Babesiosis, Balantidiasis, Blastocystosis, Coccidia, Dientamoebiasis, Entamoeba, Giardiasis, Hookworm, Isosporiasis, Leishmaniasis, tapeworm, pneumocystis carnii pneumonia, leishmaniasis, Primary amoebic meningoencephalitis, Rhinosporidiosis, Sarcocystis, Toxoplasmosis, cryptosporidiosis, schistosomiasis, trypanosome or African trypanosomiasis or sleeping sickness infection, Chagas disease, Cestoda or tapeworm infection, Diphyllobothriasis, Echinococcosis, Hymenolepiasis, Taenia saginata, Taenia solium, Bertielliasis, Sparganosis, Clonorchiasis, liver fluke infection (such as lonorchis sinensis, Dicrocoelium dendriticum (lancet liver fluke), Microcoelium hospes, Fasciola hepatica (the “sheep liver fluke”), Fascioloides magna (the “giant liver fluke”), Fasciola gigantica, Fasciola jacksoni, Metorchis conjunctus, Metorchis albidus, Protofasciola robusta, Parafasciolopsis fasciomorphae, Opisthorchis viverrini (Southeast Asian liver fluke), Opisthorchis felineus (cat liver fluke) and Opisthorchis guayaquilensis), Paragonimiasis, Schistosomiasis, Schistosoma mansoni, Urinary schistosomiasis, Asian intestinal schistosomiasis, Swimmer's itch, Ancylostomiasis, Angiostrongyliasis, Anisakis, Ascaris lumbricoides, Baylisascaris procyonis, lymphatic filariasis, Guinea worm or Dracunculiasis, Dracunculus medinensis, Pinworm or Enterobiasis, Enterobius vermicularis, Enterobius gregorii, Halicephalobiasis, Halicephalobus gingivalis, Loa loa filariasis, Mansonelliasis, Filariasis, Mansonella streptocerca, River blindness or Onchocerciasis, Onchocerca volvulus, Strongyloidiasis or Parasitic pneumonia, Strongyloides stercoralis, Thelaziasis, Thelazia californiensis, Thelazia callipaeda, Amiota (Phortica) variegata, Phortica okadai, Toxocariasis, Toxocara canis, Toxocara cati, Trichinosis, Trichinella spiralis, Trichinella britovi, Trichinella nelsoni, Trichinella nativa, Whipworm, Trichuris trichiura, Trichuris vulpis, lephantiasis Lymphatic filariasis, Wuchereria bancrofti, Acanthocephaliasis, Archiacanthocephala, Moniliformis moniliformis, Halzoun Syndrome, Linguatula serrata, Myiasis, Oestroidea, Calliphoridae, Sarcophagidae, and Tunga penetrans.

Of the foregoing, a significant parasitic disease wherein the use of GSK-3 inhibitors should be useful in therapy is schistosomiasis, a parasitic disease caused by several species of trematode of genus Schistosoma which affects almost 240 million people worldwide, and more than 700 million people live in endemic area; visceral leishmaniasis (VL), the most severe form of leishmaniasis (James et al., 2006). Leishmaniasis which is caused by protozoan parasites of the Leishmania genus and is responsible for the second greatest number of parasitically caused deaths the world (Desjeux, 2001), wherein the parasite migrates to the internal organs such as liver, spleen (hence ‘visceral’), and bone marrow. If left untreated, it causes death of the host. Of particular concern, according to the World Health Organization (WHO), is the emerging problem of HIVNL co-infection.

Another significant parasitic disease wherein the use of GSK-3 inhibitors should be useful in therapy is trichinosis, Trichinosis, also called trichinellosis, or trichiniasis, is a parasitic disease caused by eating raw or undercooked pork or wild game infected with the larvae of a species of roundworm Trichinella spiralis, commonly called the trichina worm. There are eight Trichinella species; five are encapsulated and three are not. Pozio, E., & Murrell, D. K. (2006). Systematics and Epidemiology of Trichinella. Advances in Parasitology, 63, 368-439. Only three Trichinella species are known to cause trichinosis: T. spiralis, T. nativa, and T. britovi.

Yet another significant parasitic disease which may be treated with GSK-3 inhibitors according to the invention is Chagas' disease or American trypanosomiasis, it is a tropical parasitic disease caused by the flagellate protozoan Trypanosoma cruzi. T. cruzi is commonly transmitted to humans and other mammals by an insect vector, the blood-sucking “kissing bugs” of the subfamily Triatominae (family Reduviidae), most commonly from species belonging to the Triatoma, Rhodnius, and Panstrongylus genera. [1 The symptoms of Chagas disease vary over the course of an infection. In the early, acute stage, symptoms are mild and usually produce no more than local swelling at the site of infection. The initial acute phase is responsive to anti-parasitic treatments, with 60-90% cure rates. After 4-8 weeks, individuals with active infections enter the chronic phase of Chagas disease, which is asymptomatic for 60-80% of chronically infected individuals through their lifetime.

Still another significant parasitic disease which may be treated with GSK-3 inhibitors according to the invention is African trypanosomiasis (sleeping sickness, African lethargy, or Congo trypanosomiasis) is a parasitic disease caused by protozoa of the species Trypanosoma brucei and transmitted by the tsetse fly (Morrison et al., 1983; Murray et al., 1982). Two subspecies infect humans, T.b. gambiense and T.b. rhodesiense and the disease is endemic in some regions of sub-Saharan Africa.

The subject GSK-3 inhibitors when treating a parasitic infection, such compounds may be used as a monotherapy, but more typically will be administered as part of a therapeutic regimen that includes the administration of other actives such as antibiotics, antiviral agents, anti-fungal agents or anti-parasitic agents.

Also, any of the afore-mentioned therapeutic regimens for treating infection may include the administration of other immune modulators such as afore-mentioned, and may further include the administration of therapeutic or prophylactic vaccines that may include an immune adjuvant and optionally an antigen specific to the infectious agent, e.g., a specific virus, bacteria, yeast or fungi or parasite.

Examples of such anti-parasitic agents include azole or nitro derivatives, such as benznidazole or nifurtimox; quinine, clindamycin, amebicides, metronidazole, Trimethoprim/sulfamethoxazole, Mediterranean liposomal amphotericin B, pentavalent antimonial, paromomycin, Miltefosine, Chloroquine, Amodiaquine, Pyrimethamine, Proguanil, Sulfonamides, Mefloquine, Atovaquone, Primaquine, Artemisinin and derivatives, Halofantrine, Doxycycline, Sulfadiazine, folic acid, Spiramycin, Atovaquone, nifurtimox, pentamidine, suramin, eflornithine, melarsoprol, mebendazole, praziquantel, albendazole, tinidazole, quinacrine, furazolidone and nitazoxanide, and combinations of any of the foregoing.

### Use of GSK-3 Activators to Inhibit T Cell Immunity

Another aspect of the invention relates to the use of compounds which promote the expression and/or activation of at least one isoform of GSK-3 to inhibit T cell immunity in subjects in need thereof, especially individuals where T cell function is abnormal or exacerbated such as in allergy, autoimmunity or inflammation. As GSK-3 promotes PD-1 expression and inhibits Tbet, which have been reported to suppress T_{H}1 and CD4⁺ or CD8⁺ T cell immunity, the use of compounds that promote GSK-3 activity and enhance PD-1 expression or inhibit Tbet expression should inhibit T cell immunity. Examples of compounds that promote GSK-3 activation are known and include those that promote tyrosine phosphorylation, such as by Pyk2, Fyn, Src, and Csk, octreotide, lysophosphatidic acid, leucine-rich repeat kinase 2 (LRRK2), 6-hydroxydopamine, and sphingolipids such as psychosine.

These methods may comprise a monotherapy, but more typically will comprise the administration of other actives such as immunosuppressants, antiinflammatories, antihistamines or antiallergic agents such immunosuppressive drugs (e.g., rapamycin, cyclosporine A, or FK506). Such agents may include small molecules or may comprise biologics such as antibodies and fusion proteins which agonize or antagonize the effects of specific receptors expressed on T cells, or may comprise cytokine receptor agonists or antagonists, e.g., TNF or IL-6 antagonists.

For example, this may include antibodies and fusion proteins which modulate any of the B7/CD28 or TNF/TNFR family members previously identified. In particular, this aspect of the invention may include the administration of compounds that promote or agonize PD-1 such as agonistic PD-1 antibodies or PD-L1 or PD-L2 fusion proteins. The use thereof may result in a synergistic effect on PD-1 expression or activity and thereby result in a synergistic suppressive effect on T cell immunity.

Examples of autoimmune, inflammatory disease treatable by the invention include Acid Reflux/Heartburn, Acne, Acne Vulgaris, Allergies and Sensitivities, Alzheimer's Disease, Asthma, Atherosclerosis and Vascular Occlusive Disease, optionally Atherosclerosis, Ischemic Heart Disease, Myocardial Infarction, Stroke, Peripheral Vascular Disease, or Vascular Stent Restenosis, Autoimmune Diseases, Bronchitis, Cancer, Carditis, Cataracts, Celiac Disease, Chronic Pain, Chronic Prostatitis, Cirrhosis, Colitis, Connective Tissue Diseases, optionally Systemic Lupus Erythematosus, Systemic Sclerosis, Polymyositis, Dermatomyositis, or Sjögren's Syndrome, Corneal Disease, Crohn's Disease, Crystal Arthropathies, optionally Gout, Pseudogout, Calcium Pyrophosphate Deposition Disease, Dementia, Dermatitis, Diabetes, Dry Eyes, Eczema, Edema, Emphysema, Fibromyalgia, Gastroenteritis, Gingivitis, Glomerulonephritis, Heart Disease, Hepatitis, High Blood Pressure, Hypersensitivities, Inflammatory Bowel Diseases, Inflammatory Conditions including Consequences of Trauma or Ischaemia, Insulin Resistance, Interstitial Cystitis, Iridocyclitis, Iritis, Joint Pain, Arthritis, Lyme Disease, Metabolic Syndrome (Syndrome X), Multiple Sclerosis, Myositis, Nephritis, Obesity, Ocular Diseases including Uveitis, Osteopenia, Osteoporosis, Parkinson's Disease, Pelvic Inflammatory Disease, Periodontal Disease, Polyarteritis, Polychondritis, Polymyalgia Rheumatica, Psoriasis, Reperfusion Injury, Rheumatic Arthritis, Rheumatic Diseases, Rheumatoid Arthritis, Osteoarthritis, or Psoriatic Arthritis, Rheumatoid Arthritis, Sarcoidosis, Scleroderma, Sinusitis, Sjögren's Syndrome, Spastic Colon, Spondyloarthropathies, optionally Ankylosing Spondylitis, Reactive Arthritis, or Reiter's Syndrome, Systemic Candidiasis, Tendonitis, Transplant Rejection, UTI's, Vaginitis, Vascular Diseases including Atherosclerotic Vascular Disease, Vasculitides, Polyarteritis Nodosa, Wegener's Granulomatosis, Churg-Strauss Syndrome, or vasculitis, acquired immune deficiency syndrome (AIDS), acquired splenic atrophy, acute anterior uveitis, Acute Disseminated Encephalomyelitis (ADEM), acute gouty arthritis, acute necrotizing hemorrhagic leukoencephalitis, acute or chronic sinusitis, acute purulent meningitis (or other central nervous system inflammatory disorders), acute serious inflammation, Addison's disease, adrenalitis, adult onset diabetes mellitus (Type II diabetes), adult-onset idiopathic hypoparathyroidism (AOIH), Agammaglobulinemia, agranulocytosis, vasculitides, including vasculitis, optionally, large vessel vasculitis, optionally, polymyalgia rheumatica and giant cell (Takayasu's) arthritis, allergic conditions, allergic contact dermatitis, allergic dermatitis, allergic granulomatous angiitis, allergic hypersensitivity disorders, allergic neuritis, allergic reaction, alopecia greata, alopecia totalis, Alport's syndrome, alveolitis, optionally allergic alveolitis or fibrosing alveolitis, Alzheimer's disease, amyloidosis, amylotrophic lateral sclerosis (ALS; Lou Gehrig's disease), an eosinophil-related disorder, optionally eosinophilia, anaphylaxis, ankylosing spondylitis, angiectasis, antibody-mediated nephritis, Anti-GBM/Anti-TBM nephritis, antigen-antibody complex-mediated diseases, antiglomerular basement membrane disease, anti-phospholipid antibody syndrome, antiphospholipid syndrome (APS), aphthae, aphthous stomatitis, aplastic anemia, arrhythmia, arteriosclerosis, arteriosclerotic disorders, arthritis, optionally rheumatoid arthritis such as acute arthritis, or chronic rheumatoid arthritis, arthritis chronica progrediente, arthritis deformans, ascariasis, aspergilloma, granulomas containing eosinophils, aspergillosis, aspermiogenese, asthma, optionally asthma bronchiale, bronchial asthma, or auto-immune asthma, ataxia telangiectasia, ataxic sclerosis, atherosclerosis, autism, autoimmune angioedema, autoimmune aplastic anemia, autoimmune atrophic gastritis, autoimmune diabetes, autoimmune disease of the testis and ovary including autoimmune orchitis and oophoritis, autoimmune disorders associated with collagen disease, autoimmune dysautonomia, autoimmune ear disease, optionally autoimmune inner ear disease (AGED), autoimmune endocrine diseases including thyroiditis such as autoimmune thyroiditis, autoimmune enteropathy syndrome, autoimmune gonadal failure, autoimmune hearing loss, autoimmune hemolysis, Autoimmune hepatitis, autoimmune hepatological disorder, autoimmune hyperlipidemia, autoimmune immunodeficiency, autoimmune inner ear disease (AIED), autoimmune myocarditis, autoimmune neutropenia, autoimmune pancreatitis, autoimmune polyendocrinopathies, autoimmune polyglandular syndrome type I, autoimmune retinopathy, autoimmune thrombocytopenic purpura (ATP), autoimmune thyroid disease, autoimmune urticaria, autoimmune-mediated gastrointestinal diseases, Axonal & neuronal neuropathies, Balo disease, Behçet's disease, benign familial and ischemia-reperfusion injury, benign lymphocytic angiitis, Berger's disease (IgA nephropathy), bird-fancier's lung, blindness, Boeck's disease, bronchiolitis obliterans (non-transplant) vs NSIP, bronchitis, bronchopneumonic aspergillosis, Bruton's syndrome, bullous pemphigoid, Caplan's syndrome, Cardiomyopathy, cardiovascular ischemia, Castleman's syndrome, Celiac disease, celiac sprue (gluten enteropathy), cerebellar degeneration, cerebral ischemia, and disease accompanying vascularization, Chagas disease, channelopathies, optionally epilepsy, channelopathies of the CNS, chorioretinitis, choroiditis, an autoimmune hematological disorder, chronic active hepatitis or autoimmune chronic active hepatitis, chronic contact dermatitis, chronic eosinophilic pneumonia, chronic fatigue syndrome, chronic hepatitis, chronic hypersensitivity pneumonitis, chronic inflammatory arthritis, Chronic inflammatory demyelinating polyneuropathy (CIDP), chronic intractable inflammation, chronic mucocutaneous candidiasis, chronic neuropathy, optionally IgM polyneuropathies or IgM-mediated neuropathy, chronic obstructive airway disease, chronic pulmonary inflammatory disease, Chronic recurrent multifocal osteomyelitis (CRMO), chronic thyroiditis (Hashimoto's thyroiditis) or subacute thyroiditis, Churg-Strauss syndrome, cicatricial pemphigoid/benign mucosal pemphigoid, CNS inflammatory disorders, CNS vasculitis, Coeliac disease, Cogan's syndrome, cold agglutinin disease, colitis polyposa, colitis such as ulcerative colitis, colitis ulcerosa, collagenous colitis, conditions involving infiltration of T cells and chronic inflammatory responses, congenital heart block, congenital rubella infection, Coombs positive anemia, coronary artery disease, Coxsackie myocarditis, CREST syndrome (calcinosis, Raynaud's phenomenon), Crohn's disease, cryoglobulinemia, Cushing's syndrome, cyclitis, optionally chronic cyclitis, heterochronic cyclitis, iridocyclitis, or Fuch's cyclitis, cystic fibrosis, cytokine-induced toxicity, deafness, degenerative arthritis, demyelinating diseases, optionally autoimmune demyelinating diseases, demyelinating neuropathies, dengue, dermatitis herpetiformis and atopic dermatitis, dermatitis including contact dermatitis, dermatomyositis, dermatoses with acute inflammatory components, Devic's disease (neuromyelitis optica), diabetic large-artery disorder, diabetic nephropathy, diabetic retinopathy, Diamond Blackfan anemia, diffuse interstitial pulmonary fibrosis, dilated cardiomyopathy, discoid lupus, diseases involving leukocyte diapedesis, Dressler's syndrome, Dupuytren's contracture, echovirus infection, eczema including allergic or atopic eczema, encephalitis such as Rasmussen's encephalitis and limbic and/or brainstem encephalitis, encephalomyelitis, optionally allergic encephalomyelitis or encephalomyelitis allergica and experimental allergic encephalomyelitis (EAE), endarterial hyperplasia, endocarditis, endocrine ophthalmopathy, endometriosis. endomyocardial fibrosis, endophthalmia phacoanaphylactica, endophthalmitis, enteritis allergica, eosinophilia-myalgia syndrome, eosinophilic fascitis, epidemic keratoconjunctivitis, epidermolysis bullosa acquisita (EBA), episclera, episcleritis, Epstein-Barr virus infection, erythema elevatum et diutinum, erythema multiforme, erythema nodosum leprosum, erythema nodosum, erythroblastosis fetalis, esophageal dysmotility, Essential mixed cryoglobulinemia, ethmoid, Evan's syndrome, Experimental Allergic Encephalomyelitis (EAE), Factor VIII deficiency, farmer's lung, febris rheumatica, Felty's syndrome, fibromyalgia, fibrosing alveolitis, filariasis, focal segmental glomerulosclerosis (FSGS), food poisoning, frontal, gastric atrophy, giant cell arthritis (temporal arthritis), giant cell hepatitis, giant cell polymyalgia, glomerulonephritides, glomerulonephritis (GN) with and without nephrotic syndrome such as chronic or acute glomerulonephritis (e.g., primary GN), Goodpasture's syndrome, gouty arthritis, granulocyte transfusion-associated syndromes, granulomatosis including lymphomatoid granulomatosis, granulomatosis with polyangiitis (GPA), granulomatous uveitis, Grave's disease, Guillain-Barre syndrome, gutatte psoriasis, hemoglobinuria paroxysmatica, Hamman-Rich's disease, Hashimoto's disease, Hashimoto's encephalitis, Hashimoto's thyroiditis, hemochromatosis, hemolytic anemia or immune hemolytic anemia including autoimmune hemolytic anemia (AIHA), hemolytic anemia, hemophilia A, Henoch-Schönlein purpura, Herpes gestationis, human immunodeficiency virus (HIV) infection, hyperalgesia, hypogammaglobulinemia, hypogonadism, hypoparathyroidism, idiopathic diabetes insipidus, idiopathic facial paralysis, idiopathic hypothyroidism, idiopathic IgA nephropathy, idiopathic membranous GN or idiopathic membranous nephropathy, idiopathic nephritic syndrome, idiopathic pulmonary fibrosis, idiopathic sprue, Idiopathic thrombocytopenic purpura (ITP), IgA nephropathy, IgE-mediated diseases, optionally anaphylaxis and allergic or atopic rhinitis, IgG4-related sclerosing disease, ileitis regionalis, immune complex nephritis, immune responses associated with acute and delayed hypersensitivity mediated by cytokines and T-lymphocytes, immune-mediated GN, immunoregulatory lipoproteins, including adult or acute respiratory distress syndrome (ARDS), Inclusion body myositis, infectious arthritis, infertility due to antispermatozoan antibodies, inflammation of all or part of the uvea, inflammatory bowel disease (IBD) inflammatory hyperproliferative skin diseases, inflammatory myopathy, insulin-dependent diabetes (type1), insulitis, Interstitial cystitis, interstitial lung disease, interstitial lung fibrosis, iritis, ischemic re-perfusion disorder, joint inflammation, Juvenile arthritis, juvenile dermatomyositis, juvenile diabetes, juvenile onset (Type I) diabetes mellitus, including pediatric insulin-dependent diabetes mellitus (IDDM), juvenile-onset rheumatoid arthritis, Kawasaki syndrome, keratoconjunctivitis sicca, kypanosomiasis, Lambert-Eaton syndrome, leishmaniasis, leprosy, leucopenia, leukocyte adhesion deficiency, Leukocytoclastic vasculitis, leukopenia, lichen planus, lichen sclerosus, ligneous conjunctivitis, linear IgA dermatosis, Linear IgA disease (LAD), Loffler's syndrome, lupoid hepatitis, lupus (including nephritis, cerebritis, pediatric, non-renal, extra-renal, discoid, alopecia), Lupus (SLE), lupus erythematosus disseminatus, Lyme arthritis, Lyme disease, lymphoid interstitial pneumonitis, malaria, male and female autoimmune infertility, maxillary, medium vessel vasculitis (including Kawasaki's disease and polyarteritis nodosa), membrano- or membranous proliferative GN (MPGN), including Type I and Type II, and rapidly progressive GN, membranous GN (membranous nephropathy), Meniere's disease, meningitis, microscopic colitis, microscopic polyangiitis, migraine, minimal change nephropathy, Mixed connective tissue disease (MCTD), mononucleosis infectiosa, Mooren's ulcer, Mucha-Habermann disease, multifocal motor neuropathy, multiple endocrine failure, multiple organ injury syndrome such as those secondary to septicemia, trauma or hemorrhage, multiple organ injury syndrome, multiple sclerosis (MS) such as spino-optical MS, multiple sclerosis, mumps, muscular disorders, myasthenia gravis such as thymoma-associated myasthenia gravis, myasthenia gravis, myocarditis, myositis, narcolepsy, necrotizing enterocolitis, and transmural colitis, and autoimmune inflammatory bowel disease, necrotizing, cutaneous, or hypersensitivity vasculitis, neonatal lupus syndrome (NLE), nephrosis, nephrotic syndrome, neurological disease, neuromyelitis optica (Devic's), neuromyelitis optica, neuromyotonia, neutropenia, non-cancerous lymphocytosis, nongranulomatous uveitis, non-malignant thymoma, ocular and orbital inflammatory disorders, ocular cicatricial pemphigoid, oophoritis, ophthalmia symphatica, opsoclonus myoclonus syndrome (OMS), opsoclonus or opsoclonus myoclonus syndrome (OMS), and sensory neuropathy, optic neuritis, orchitis granulomatosa, osteoarthritis, palindromic rheumatism, pancreatitis, pancytopenia, PANDAS (Pediatric Autoimmune Neuropsychiatric Disorders Associated with Streptococcus), paraneoplastic cerebellar degeneration, paraneoplastic syndrome, paraneoplastic syndromes, including neurologic paraneoplastic syndromes, optionally Lambert-Eaton myasthenic syndrome or Eaton-Lambert syndrome, parasitic diseases such as Leishmania, paroxysmal nocturnal hemoglobinuria (PNH), Parry Romberg syndrome, pars planitis (peripheral uveitis), Parsonnage-Turner syndrome, parvovirus infection, pemphigoid such as pemphigoid bullous and skin pemphigoid, pemphigus (including pemphigus vulgaris), pemphigus erythematosus, pemphigus foliaceus, pemphigus mucus-membrane pemphigoid, pemphigus, peptic ulcer, periodic paralysis, peripheral neuropathy, perivenous encephalomyelitis, pernicious anemia (anemia perniciosa), pernicious anemia, phacoantigenic uveitis, pneumonocirrhosis, POEMS syndrome, polyarteritis nodosa, Type I, II, & III, polyarthritis chronica primaria, polychondritis (e.g., refractory or relapsed polychondritis), polyendocrine autoimmune disease, polyendocrine failure, polyglandular syndromes, optionally autoimmune polyglandular syndromes (or polyglandular endocrinopathy syndromes), polymyalgia rheumatica, polymyositis, polymyositis/dermatomyositis, polyneuropathies, polyradiculitis acuta, post-cardiotomy syndrome, posterior uveitis, or autoimmune uveitis, postmyocardial infarction syndrome, postpericardiotomy syndrome, post-streptococcal nephritis, post-vaccination syndromes, presenile dementia, primary biliary cirrhosis, primary hypothyroidism, primary idiopathic myxedema, primary lymphocytosis, which includes monoclonal B cell lymphocytosis, optionally benign monoclonal gammopathy and monoclonal garnmopathy of undetermined significance, MGUS, primary myxedema, primary progressive MS (PPMS), and relapsing remitting MS (RRMS), primary sclerosing cholangitis, progesterone dermatitis, progressive systemic sclerosis, proliferative arthritis, psoriasis such as plaque psoriasis, psoriasis, psoriatic arthritis, pulmonary alveolar proteinosis, pulmonary infiltration eosinophilia, pure red cell anemia or aplasia (PRCA), pure red cell aplasia, purulent or nonpurulent sinusitis, pustular psoriasis and psoriasis of the nails, pyelitis, pyoderma gangrenosum, Quervain's thyroiditis, Raynaud's phenomenon, reactive arthritis, recurrent abortion, reduction in blood pressure response, reflex sympathetic dystrophy, refractory sprue, Reiter's disease or syndrome, relapsing polychondritis, reperfusion injury of myocardial or other tissues, reperfusion injury, respiratory distress syndrome, restless legs syndrome, retinal autoimmunity, retroperitoneal fibrosis, Reynaud's syndrome, rheumatic diseases, rheumatic fever, rheumatism, rheumatoid arthritis, rheumatoid spondylitis, rubella virus infection, Sampter's syndrome, sarcoidosis, schistosomiasis, Schmidt syndrome, SCID and Epstein-Barr virus-associated diseases, sclera, scleritis, sclerodactyl, scleroderma, optionally systemic scleroderma, sclerosing cholangitis, sclerosis disseminata, sclerosis such as systemic sclerosis, sensoneural hearing loss, seronegative spondyloarthritides, Sheehan's syndrome, Shulman's syndrome, silicosis, Sjögren's syndrome, sperm & testicular autoimmunity, sphenoid sinusitis, Stevens-Johnson syndrome, stiff-man (or stiff-person) syndrome, subacute bacterial endocarditis (SBE), subacute cutaneous lupus erythematosus, sudden hearing loss, Susac's syndrome, Sydenham's chorea, sympathetic ophthalmia, systemic lupus erythematosus (SLE) or systemic lupus erythematodes, cutaneous SLE, systemic necrotizing vasculitis, ANCA-associated vasculitis, optionally Churg-Strauss vasculitis or syndrome (CSS), tabes dorsalis, Takayasu's arteritis, telangiectasia, temporal arteritis/Giant cell arteritis, thromboangiitis ubiterans, thrombocytopenia, including thrombotic thrombocytopenic purpura (TTP) and autoimmune or immune-mediated thrombocytopenia such as idiopathic thrombocytopenic purpura (ITP) including chronic or acute ITP, thrombocytopenic purpura (TTP), thyrotoxicosis, tissue injury, Tolosa-Hunt syndrome, toxic epidermal necrolysis, toxic-shock syndrome, transfusion reaction, transient hypogammaglobulinemia of infancy, transverse myelitis, traverse myelitis, tropical pulmonary eosinophilia, tuberculosis, ulcerative colitis, undifferentiated connective tissue disease (UCTD), urticaria, optionally chronic allergic urticaria and chronic idiopathic urticaria, including chronic autoimmune urticaria, uveitis, anterior uveitis, uveoretinitis, valvulitis, vascular dysfunction, vasculitis, vertebral arthritis, vesiculobullous dermatosis, vitiligo, Wegener's granulomatosis (Granulomatosis with Polyangiitis (GPA)), Wiskott-Aldrich syndrome, or x-linked hyper IgM syndrome.

### Screening Methods

The invention further provides methods of screening for a PD-1 modulator comprising the steps of:
(i) incubating purified GSK-3 alpha or beta with a test molecule or chemical;
(ii) measuring GSK-3 kinase activity in said sample; and
(iii) comparing the level of GSK-3 activity in the sample to the level of GSK-3 activity in a control sample in which the test molecule is absent. A change in the level of GSK-3 activity relative to the control is indicative of a modulatory effect of the chemical on GSK3 and PD-1.

In one embodiment, a decrease in the level of GSK-3 activity is indicative of decreased transcription and expression of PD-1.

In another embodiment, an increase in the level of GSK3 activity is indicative of increased transcription and expression of PD-1.

As the inventor has discovered that GSK-3 inactivation reduces PD-1 expression, a GSK-3 inhibitor can be used to screen for agents that may be identified as novel PD-1 modulators. When the level of GSK-3 activity is decreased, i.e. GSK-3 inactivation, this indicates that PD-1 expression is suppressed. The data can also be used to show that alternatively PD-1 activity and/or expression can be monitored to screen for agents that may be identified as novel GSK-3 inhibitors. According to a further aspect of the invention, there is provided a PD-1 modulator identified by the method of screening defined herein.

In one embodiment, a decrease in the level of GSK-3 activity is indicative of decreased transcription and expression of PD-1.

In another embodiment, an increase in the level of GSK3 activity is indicative of increased transcription and expression of PD-1.

The invention further provides methods of screening for a Tbet modulator comprising the steps of:
(i) incubating purified GSK-3 alpha or beta with a test molecule or chemical;
(ii) measuring GSK-3 kinase activity in said sample; and
(iii) comparing the level of GSK-3 activity in the sample to the level of GSK-3 activity in a control sample in which the test molecule is absent. A change in the level of GSK-3 activity relative to the control is indicative of a modulatory effect of the chemical on GSK3 and Tbet.

In one embodiment, a decrease in the level of GSK-3 activity is indicative of increased transcription and expression of Tbet.

In another embodiment, an increase in the level of GSK3 activity is indicative of decreased transcription and expression of Tbet.

As the inventor has discovered that GSK-3 inactivation increases Tbet expression, a GSK-3 inhibitor can be used to screen for agents that may be identified as novel Tbet modulators. When the level of GSK-3 activity is decreased, i.e. GSK-3 inactivation, this indicates that Tbet expression is increased. The data can also be used to show that alternatively Tbet activity and/or expression can be monitored to screen for agents that may be identified as novel GSK-3 inhibitors. According to a further aspect of the invention, there is provided a Tbet modulator identified by the method of screening defined herein.

In one embodiment, a decrease in the level of GSK-3 activity is indicative of increased transcription and expression of Tbet.

In another embodiment, an increase in the level of GSK3 activity is indicative of decreased transcription and expression of Tbet.

The invention further provides methods of screening for the efficacy of an anti-PD-1 in immunotherapy by measuring the effect of an antibody on the transcription of PD-1. As the inventor has discovered that anti-PD-1 ligation of cells reduces PD-1 transcription, a polymerase chain reaction assay or other established means for measuring the transcription of PD-1 such as an EMSA assay are used to screen for agents that may be identified as novel PD-1 modulators. Methods could also involve screening for antibodies to CTLA-4 in the same manner that the inventor has shown can reduce PD-1 transcription and/or the screening anti-CTLA-4 and antibodies to other receptors that can cooperate with a given anti-PD-1 antibody is reducing PD-1 expression.

As an example, the methods would include Incubating cells expressing PD-1 with an anti-PD-1 antibody for various times and with different concentrations (i.e. a titration of antibody concentrations). Such antibodies can be manufactured using a partial portion of the extracellular region of PD-1 using well-known production methods for the generation of monoclonal antibodies or antiserum. Antibody can be prepared as a full length antibody, a single chain antibody, a scFv antibody, an Fab′ antibody fragment, F(ab′)2 or fragments of a protein with the capability to bind to the receptor.

Anti-PD-1 also may be combined with other antibodies such as anti-CTLA-4 to measure effects on PD-1 transcription. Given the precedent of anti-PD-1 ligation inhibiting PD-1 transcription and given that anti-CTLA-4 is shown to also inhibit PD-1 transcription, antibodies to CTLA-4 and other co-receptors or cytokines can be used to measure effects on PD-1 transcription, either alone or in combination with anti-PD1. Antibodies to CTLA-4 and other co-receptors such as LAG-3, VISTA and others may also be screen for an ability to inhibit PD-1 transcription based on the precedent outlined within. The antibody may be added alone or in combination to a cell culture with cells expressing PD-1.

Secondary antibody such as a monoclonal antibody to the Fc region of anti-PD-1 or another antibody may be used to crosslink or cluster the antibody or receptor complexes.

Reverse transcription is one method that is performed to measure PD-1 transcription using the RNA polymerase chain reaction (PCR) using established procedures. Quantitative real-time PCR on cDNA generated from the reverse transcription of purified RNA using established procedures. Single-strand cDNA can be synthesized with an RT-PCR. mRNA expression was normalized against GAPDH expression using the standard curve method. An example of an oligo-sequence that could be used for PD-1 includes FW, 5-CCGCCTTCTGTAATGGTTTGA-3; PD-1-RV, 5-GGGCAGCTGTAT GATCTGGAA-3. Similarly for the GAPDH control would be FW, 5-CAACAGCAACTCCCAC TCTTC-3; GAPDH-RW, 5-GGTCCAGGGTT TCTTACTCCTT-3. Other approaches for measuring gene transcription and activation are well established and would include an EMSA and promoter assays.

One would measure the level of effect of anti-PD-1, a fragment of anti-PD-1 or another antibody on PD-1 transcription relative to a control sample in which the test antibody or molecule is absent. A change in the level of PD-1 transcription relative to the control is indicative of a modulatory effect of the antibody on PD-1 transcription.

In one embodiment, an effect on PD-1 transcription at the lowest antibody concentration is indicative of a more effective therapeutic anti-PD-1 antibody

In another embodiment, an effect on another antibody such as anti-CTLA-4, LAG-3, Tim-3, Vista etc. on PD-1 transcription is indicative of a therapeutic antibody.

In another embodiment, an effect on another antibody such as to CTLA-4, LAG-3, Tim-3, Vista etc. in combination with anti-PD-1 on PD-1 transcription isindicative of the synergistic or additive effect of the therapeutic antibody combination.

In another embodiment, the absence of an effect of anti-PD-1 antibody on PD-1 transcription is indicative of an anti-PD-1 antibody that can block the effects of anti-PD-1 therapeutic antibodies, or has effects exclusively due to the binding of the antibody to its receptor without affecting PD-1 transcription.

As the inventor has discovered that GSK-3 inactivation reduces PD-1 expression, a GSK-3 inhibitor can be used to screen for agents that may be identified as novel PD-1 modulators. When the level of GSK-3 activity is decreased, i.e. GSK-3 inactivation, this indicates that PD-1 expression is suppressed. The data can also be used to show that alternatively PD-1 activity and/or expression can be monitored to screen for agents that may be identified as novel GSK-3 inhibitors. According to a further aspect of the invention, there is provided a PD-1 modulator identified by the method of screening defined herein.

The following experimental examples further illustrate the invention. The Materials and Methods set forth below were used in the Examples which follow thereafter.

### Materials and Methods

### Mice

Wild-type C57BL/6 mice (i.e. B6), C57BL/6-OT-1 Tg, DO11.1 Tg and outbred ICR/CD1 mice (Taconic labs) were used throughout the majority of the study. All experiments conformed to local and national ethical regulations.

### Antibodies/Reagents

Anti-mouse CD3 (145-2C11-APC), anti-mouse CTLA-4 (UC10-4B9-PE), anti-mouse CD44-APC and anti-FasL-APC were purchased from eBioscience (UK). Unconjugated anti-PD-1 was purchased from BioXpress (New Hampshire, USA), while anti-mouse PD-1 PE (CD279) was obtained from eBioscience (UK) (J43), or Biolegend (US) (RMP1-30). Concanavalin A (Con A) was obtained from Sigma. GSK-3 inhibitors SB216763, 3-(2,4-dichlorophenyl)-4-(1-methyl-1H-indol-3-yl)-1H-pyrrole-2,5-dione], SB415286 3-(3-chloro-4-hydroxyphenylamino)-4-(2-nitrophenyl)-1H-pyrrole-2,5-dione (Abcam plc) L803-mts, AR-A014418 [N-[(4-Methoxyphenyl)methyl]-N′-(5-nitro-2-thiazolyl)urea], CHIR-99021 (CT99021) [6-(2-(4-(2,4-dichlorophenyl)-5-(4-methyl-1H-imidazol-2-yl)pyrimidin-2-ylamino) ethylamino)nicotinonitrile hydrochloride] (Tocris, R & D systems) and the thiadiazolidinone TDZD-8 [8 1,2,4-Thiadiazolidine-3,5-dione, 2-methyl-4-(phenylmethyl)] (Selleckchem, UK), AZD1080 (C19H18N4O2) (MedChemexpress, Princeton, N.J.) were obtained the enclosed sources.

### Cells and Cultures

T cells were isolated from spleens and re-suspended in RPMI 1640 medium supplemented with 10% (v/v) fetal calf serum (FCS), 2 mM L-glutamine, 100 U/ml penicillin and streptomycin, (GIBCO). In some cases, T cells were purified using T cell enrichment columns (R&D). For OVA peptide presentation of OVA antigen in vitro, primary murine T cells from T-cell receptor (TCR)-transgenic DO11.10 mice (2×10⁶/ml) were cultured in RPMI 1640 containing 10% FCS, 2 mM glutamine, 100 IU/mL penicillin, 100 g/mL streptomycin, and 50M 2-ME as outlined (Lu et al (2012) Blood 120, 4560-4570). For the generation of bone marrow derived dendritic cells, bone marrow was flushed from femurs, passed through a 40 um mesh to remove fibrous tissue and red cells were lysed as described using ACK (0.15 M NH₄Cl, 1 mM NaHCO₃, 0.1 mM EDTA, PH 7.25) (Lu et al (2012) Blood 120, 4560-4570). Cells were cultured in RPMI 1640 medium that was supplemented with 10% (v/v) FCS, 2 mM glutamine, 50 uM 2-ME, 100 U/ml penicillin/streptomycin, 20 ng/ml recombinant murine GM-CSF and 10 ng/ml interleukin 4 (1-4). On day 3 of culture, floating cells were gently removed and fresh GM-CSF and IL-4 containing medium was added. On day 7 of culture, BMDCs were induced to mature by adding 1 ug/ml LPS to the culture overnight. 2-5×10⁵ DCs were used to present OVA peptide to 2×10⁶ T-cells using established methods.

For OVA peptide presentation of antigen in vitro to OT-1 cells, T-cells (2×10⁶/ml) were activated and cytolytic T-cells (CTLs) were generated by incubation with 10 nM OVA₂₅₇₋₂₆₄ peptide (Sachem) using EL-4 cells (5×10⁵/ml) as antigen-presenting cells in the presence or absence of GSK-3 inhibitors and/or PD-1 blockade for 5 days prior to washing and analysis by FACs, PCR or cytoxicity assays using established methods.

For anti-CD3 activation of T-cells, cells were stimulated with 5 μg/ml of anti-CD3 (2C11) in RPMI medium supplemented with 10% FCS, 2 mM glutamine, 50 uM 2-ME, 100 U/ml penicillin/streptomycin for 2-4 days using established methods.

For flow cytometry, cells in RPMI 1640 at 10⁶/ml were incubated with various antibodies (1:500-1:1000 dilution from a 1 mg/ml stock) for 60 min at 4° C. Cells were then washed twice in RPMI 1640 and fixed with 1% paraformaldehyde 5 min. The presence of cells was confirmed by using forward scatter height or side scatter height n the form of a dot and contor plots. Staining was assessed for CD3, CD44, CD69, CD152 and PD-1 expression in the FL1 (APC; Allophycocyanin) or FL2 (PE; Phycoerythrin) channels using Becton Dickinson FACsCalibur or LSRFortessa cell analyzer using established methods. Non-stained cells were used a control.

### Polymerase Chain Reaction (PCR)

Single-strand cDNA was synthesized with an RT-PCR kit (Qiagen, Hilden, Germany) according to the manufacturer's instructions. Reverse transcription was performed using the RNA polymerase chain reaction (PCR) core kit (Applied Biosystems). Relative quantitative real-time PCR used SYBR green technology (Roche) on cDNA generated from the reverse transcription of purified RNA. After preamplification (95° C. for 2 min), the PCRs were amplified for 40 cycles (95° C. for 15 s and 60° C. for 60 s) in a sequence detection system (PE Prism 7000; Perkin-Elmer Applied Biosystems, USA). mRNA expression was normalized against GAPDH expression using the standard curve method.

PD-1-FW,
5-CCGCCTTCTGTAATGGTTTGA-3
PD-1-RV,
5-GGGCAGCTGTATGATCTGGAA-3
GAPDH-FW,
5-CAACAGCAACTCCCACTCTTC-3
GAPDH-RW,
5-GGTCCAGGGTTTCTTACTCCTT-3
TBET-FW,
5-GATCGTCCTGCAGTCTCTCC-3
TBET-RW,
5-AACTGTGTTCCCGAGGTGTC-3

### Cytotoxicity Assays

Cytotoxicity was assayed using a Cytotox 96 nonradioactive kit (Promega) following the instructions provided and using established methods. In brief, purified T cells were plated in 96-well plates at the effector/target ratios shown using 10⁴ EL4 (ova peptide-pulsed) target cells per well in a final volume of 200 μl per well using RPMI lacking phenol red. Target cells per well were in a final volume of 200 μl per well using RPMI lacking phenol red. Lactate dehydrogenase release was assayed after 4 h incubation at 37° C. by removal of 50 μl supernatant from each well and incubation with substrate provided for 30 min and the absorbance read at 490 nm using the Thermomax plate reader (Molecular Devices). Percentage cytotoxicity=((experimental effectorₛₚₒₙₜₐₙₑₒᵤₛ−target spontaneous)/(targetₘₐₓᵢₘᵤₘ−target spontaneous))×100. All cytotoxicity assays were reproducible in at least three independent assays (Jenkins, M R et al, 2009).

### Priming OT-1 Tg Cells In Vivo

OVA peptide (1 mg) was injected intravenously into OT-1 Tg mice with and without SB415286 (10 μg) in 100 μl of PBS. Spleens were harvested after 7 days and T cells purified. Longer experiments utilized a repeat injection on day that was reminiscent of the initial injection. Spleens were then harvested on day 14 and T cells purified.

### Intradermal Tumor Establishment in OT-1 Tg Mice

EL4 tumor cells taken from the log phase of in vitro growth were pulsed with ova peptide for 1 hr at 37 C before washing and injecting into OT-1 Tg mice (typically 3×10⁶ cells). EL4 cells were co-injected with/without SB415286 into the right flank skin and non-pulsed EL4 cells were injected into the left flank to act as a control. Tumors were clearly visible after 1 wk and grew progressively, in an encapsulated fashion. Induced tumors were measured on a daily basis using a vernier caliper (Helmich et al, 2001, J Immunol 166: 6500-6508; Quezada et al, 2010, J Exp Med 207: 637-650). Tumors and spleens were harvested on Day 10 when PCR was performed.

### Oral Administration of GSK-3 Inhibitor TDZD-8

TDZD-8 was administered to mice to achieve a dose of 2 mg/kg (as reviewed by Martinez et al 2013 Curr Top Med Chem 13, 108-1819). A stock solution 1 mg/ml was made in 1% DMSO. In one set, the 10 ug stock solution was diluted in 10 ml of water. In a control set, a comparable volume of 1% DMSO solution was added to 10 ml of water. By 48 hours, all water was consumed. Mice were then sacrificed, spleen extracted on 60 hours, cells were spun down at 1,800 for 3 min and ACK for removal of red blood cells (RBCs) treated for 2 min. Cells were then centrifuged at 1,800 rpm for 3 min and the absence of red blood cells was confirmed by the loss of red color in the cell pellet. The presence of cells was confirmed by light microscopy followed by counting of the cells that numbered between 140-160×10⁶ cells. No difference on cell numbers was observed between drug treated and untreated mice. Cells were then suspended in culture media comprised of 10% foetal calf serum (FCS), RPMI 1640 and Penn strep (xxx). Cells at 2×106 cells/ml were then plated in 24 well plates that had been pre-coated with anti-CD3 2C11 at 2 ugml. An additional stimulant of 2 ug/ml ConA was also added at 48 hours of culture. Cells recovered a stained with directly conjugated anti-PD1 and analyzed using Becton Dickinson FACsCalibur or LSRFortessa cell analyzer. Non-stained cells were used a control.

### Example 1: Incubation of T-Cells with Inhibitors of GSK3 (SB215286 or SB216763) Inhibits PD-1 Transcription and Expression and Increases Tbet Transcription

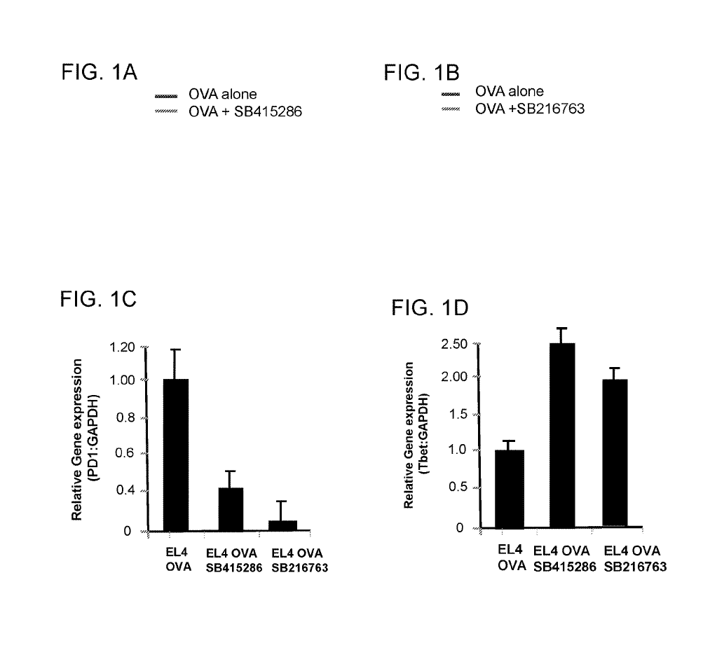

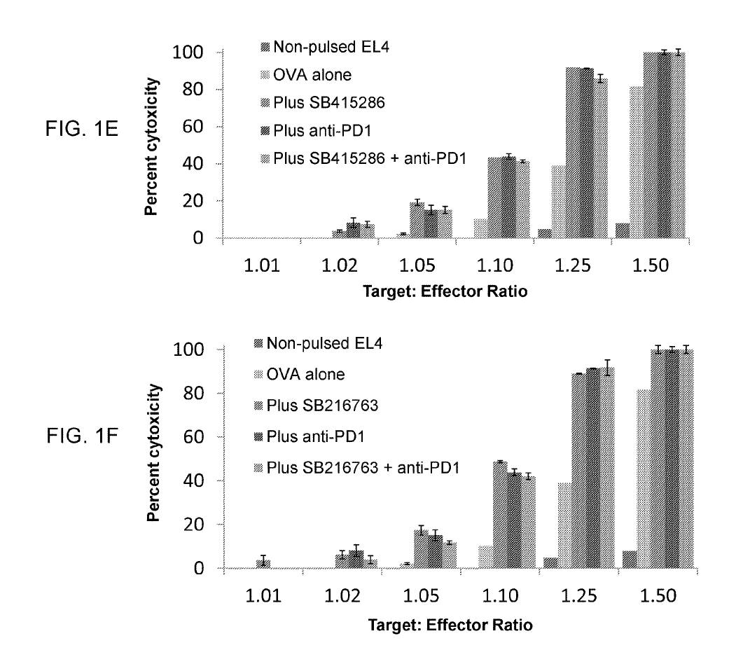

This example relates to the experiments in FIG. 1. Spleen T-cells from the MHC class I-restricted OVA specific T cell receptor (TCR) transgenic mice (OT-1) mice with a TCR specific for the SIINFEKL peptide of OVAlbumin (OVA257-264) as presented by H-2kb were incubated with OVA peptide for 7 days. OT-1 T-cells were stimulated in vitro by OVA peptide presented by EL-4 cells in the presence or absence of SB215286 or SB216763 for 3 days. Cells were then stained for FACs with APC conjugated anti-PD-1 (CD279) (a, b) or subjected to qPCR (c, d) as described in the Materials and Methods. Reverse transcription was performed using the RNA polymerase chain reaction (PCR). PCRs were amplified for 40 cycles in a sequence detection system. mRNA expression was normalized against GAPDH (Glyceraldehyde 3-phosphate dehydrogenase) expression using the standard curve method.

(a) FACS profile showing reduced PD-1 expression due to incubation with SB415286. (b) FACS profile showing reduced PD-1 expression due to incubation with SB216763. (c) Incubation with SB415286 and SB216763 decreased PD-1 transcription. (d) Incubation with SB415286 and SB216763 increased Tbet transcription. These data show that the inactivation of GSK-3 by two different inhibitors (SB415286 and SB216763) decreased PD-1 transcription/expression and increased Tbet transcription in T-cells stimulated by peptide antigen.

(e, f) shows that anti-PD-1 and GSK-3 inhibition increased CTL killing of targets to the same extent, and secondly, the addition of anti-PD-1 to SB415286 (e) or SB216763 (f) treated cells did not increase CTL function further, and vice versa. The inability of anti-PD-1 to increase the effects of SB415286 or SB216763 further and vice versa shows that GSK-3 enhancement of CTL function is due to its down-regulation of PD-1. If GSK-3 increases CTL function via another receptor or pathway, its inactivation would have potentiated the killing beyond that seen with anti-PD-1. The addition of both reagents would be additive. This was not observed. EL4-OVA targets by OT-1 CD8+ CTL that were generated for 7 days in the presence or absence of SB415286 or blocking anti-PD-1 or PDL1-Fc. (e) shows the killing by OT-1 cells incubated in the presence or absence of SB415286 and/or anti-PD-1. It shows that the inhibition of GSK-3 when present from the start of culture increases OT-1 cytolytic killing of EL4-OVA target cells. It also shows that anti-PD-1 increases the CTL function by the same degree over a range of target: effector ratios, and that anti-PD-1 does not increase further the increased killing mediated by SB415286 and vice versa. (f) shows that same effect using SB216763 to inhibit GSK-3. Identical results were obtained where anti-PD-1 did not increase killing beyond that seen with SB216763 and vice versa. These data showed that SB415286 (e) and SB216763 (f) increase CTL function due to the down-regulation of PD-1.

### Example 2: SB415286 Down-Regulation of PD-1 Expression Occurs without the Inhibition of Other Receptors

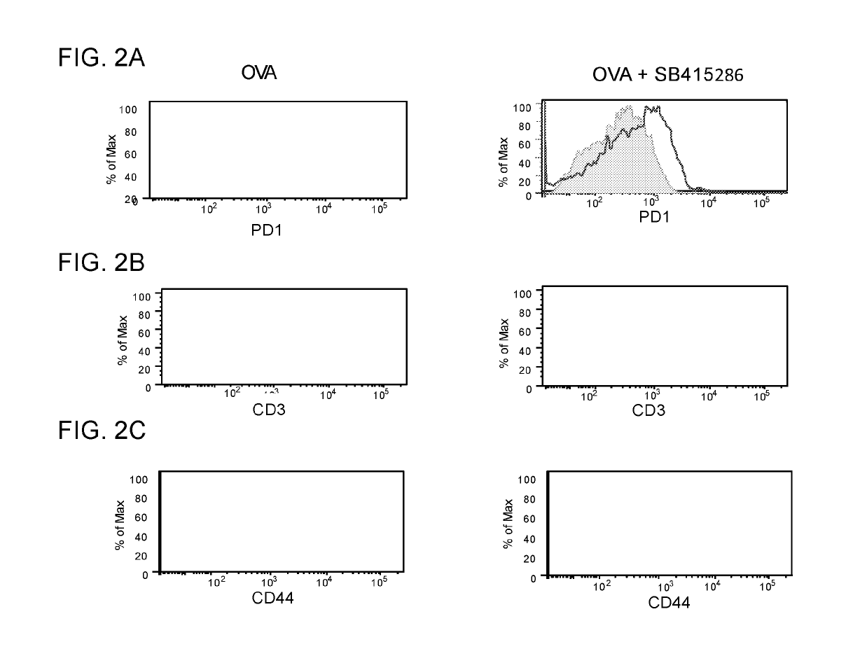

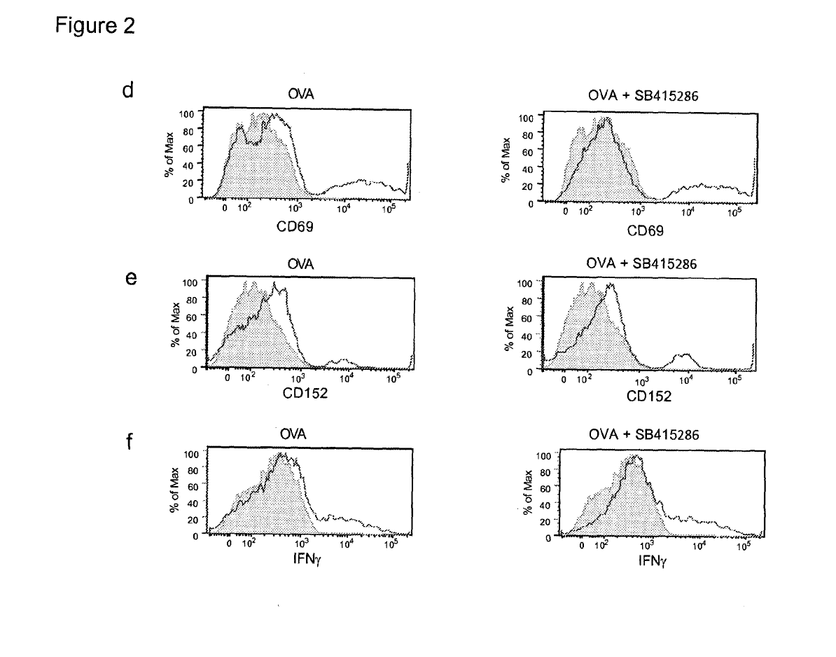

This example relates to the experiments in FIG. 2. T-cells were activated as in Example 1 and stained with antibodies to PD-1, CD3 and CD44. (a) FACS profile showing reduced PD-1 expression due to incubation with SB415286. (b) FACS profile showing unaltered CD3 expression due to incubation with SB415286. (c) FACS profile showing unaltered CD44 expression due to incubation with SB415286. The results show that SB415286 down-regulates PD-1 expression without affecting the expression of other T cell receptors CD3 and CD44.

### Example 3: SB216763 Down-Regulation of PD-1 Expression Occurs without the Inhibition of Other Receptors

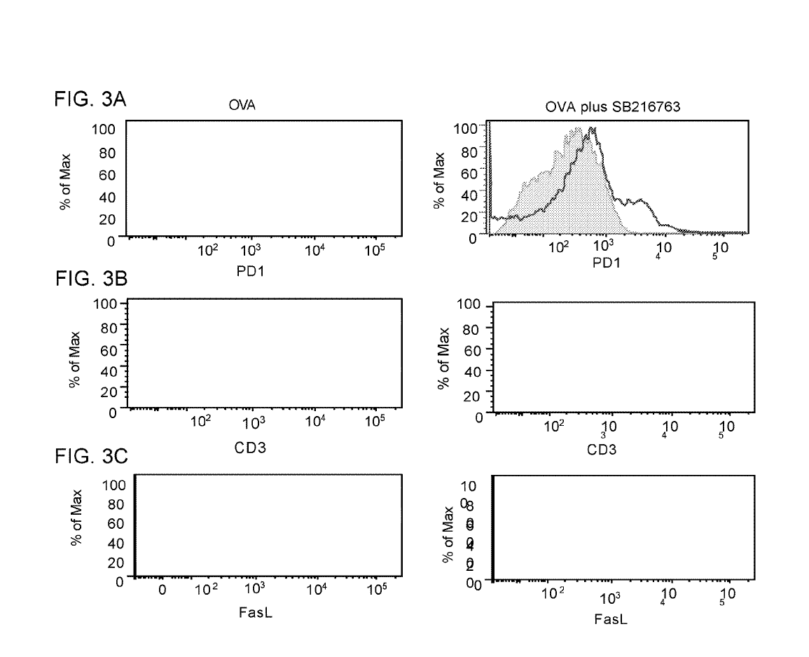

This example relates to the experiments in FIG. 3. T-cells were activated as in Example 1 and stained with antibodies to PD-1, CD3 and FasL. (a) FACS profile showing reduced PD-1 expression due to incubation with SB216763. (b) FACS profile showing unaltered CD3 expression due to incubation with SB216763. (c) FACS profile showing unaltered FasL expression due to incubation with SB216763. The results in FIG. 3 show that another inhibitor of GSK-3 SB216763 also down-regulates PD-1 expression without affecting the expression of other T cell receptors CD3 and FasL.

### Example 4: Different Structurally Distinct Inhibitors of GSK-3 Inhibit PD-1 Expression and Transcription

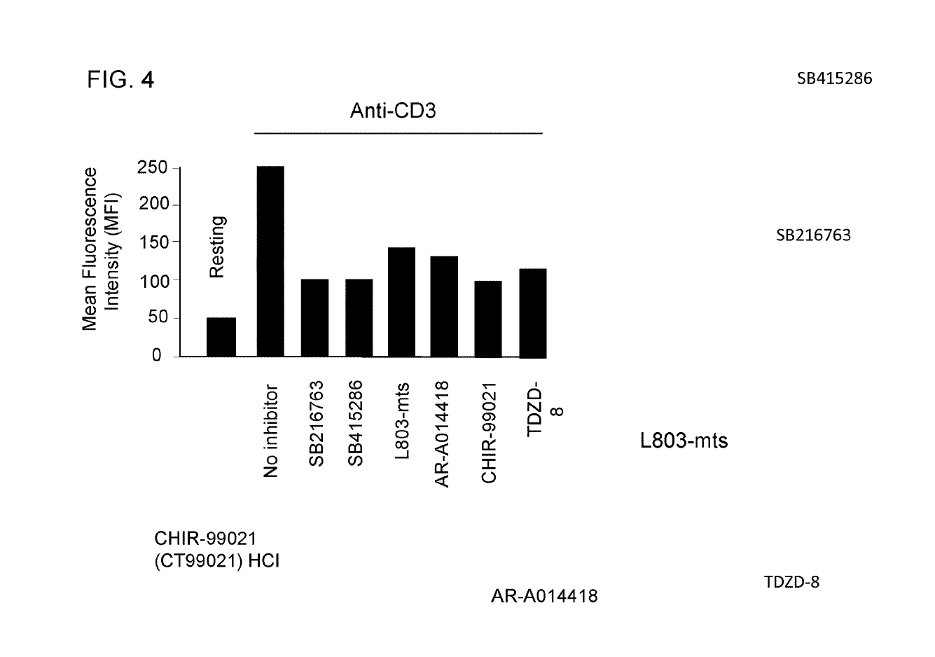

This example relates to the experiments in FIG. 4. The figure shows the effect of structurally distinct competitive and non-competitive inhibitors of GSK-3 on PD-1 expression. Primary mouse spleen T-cells were activated with either anti-CD3 (2C11) for 48 hours in the presence or absence of inhibitor followed by harvesting of cells and FACs analysis using anti-PD-1-PE (CD279; clone J43; Affymetrix eBioscience). FACS histogram showing that anti-CD3 increased the mean fluorescence intensity (MFI) of PD-1 expression by 5 fold. Further, the presence of each inhibitor reduced inhibited this increase in expression by more than 40 percent. These inhibitors included such the arylindolemaleimides SB216763, SB415286 (developed by GlaxoSmithKline) (inhibition >60%), the peptide competitor L803-mts (Kaidanovich-Beilin et al., 2004 Biol. Psychiatry 55, 781) (inhibition >60%), the amino thiazole AR-A014418 (developed by AstraZeneca) (Bhat et al 2003 J Biol Chem 46, 45937) (inhibition >45%), the purine analog, the aminopyrimidine, CHIR-99021 (CT99021) (developed by Chiron) (Bennett C N, et al. J Biol Chem, 2002, 277(34), 30998-31004) (inhibition >55%) and the small heterocyclic thiadiazolidinones (TDZD) family that includes the compound, TDZD-8 (Martinez et al., 2002; Zhu et al., 2011) (inhibition >50%). Each are structurally distinct, some are ATP competitive inhibitors such as SB216763, SB415286, while TDZD-8 is an ATP non-competitive inhibitor. The chemical structures of each inhibitor are shown on bottom and right sides of figure. Despite their structural differences, each was capable of down-regulating PD-1 expression.

### Example 5: GSK-3 Inhibition Inhibits PD-1 Expression on MLR and Con A Activated T-Cells

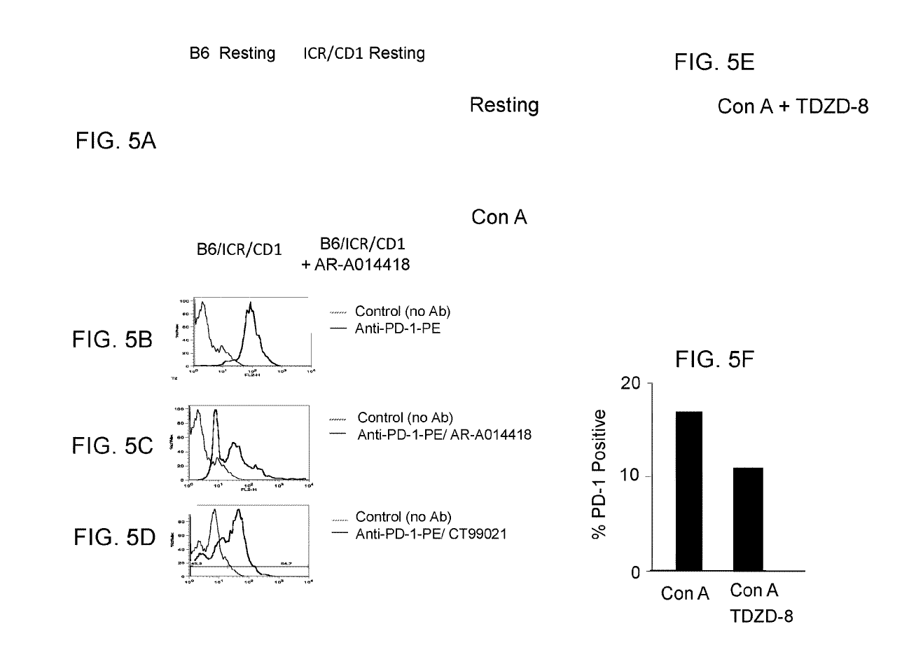

This example relates to the experiments in FIG. 5. T-cells can be stimulated in different ways. In addition to anti-CD3 stimulation (Example 4), the mixed lymphocyte reaction (MLR) activates T-cells due to the recognition of allo-histocompatibility antigens on the opposing cells. The MLR can predict an individual's response to a transplanted tissue or organ. Splenocytes from outbred ICR/CD1 mice will mount a stronger immune response to inbred C57BI/6 mice and vice versa due to a greater major histocompatibility complex (MHC) difference. Inbred C57BI/6 and outbred ICR/CD1 mouse spleen T-cells were either cultivated alone or co-cultured at equal numbers (1×10⁶/ml) for 60 hours in the presence or absence of inhibitors AR-A014418 or CT99021 followed by FACs analysis of PD-1 expression. (a) shows the bright field images of B6 or ICR/CD1 T-cells alone (upper panels), or co-cultured in the absence or presence of AR-A014418 (lower panels; arrow points to cell clusters). Both B6/ICR/CD1 and B6/ICR/CD1+AR-A014418 cultures showed the presence of clusters. Cell clustering as well as the expression of the activation antigen PD-1 (i.e. whose expression depends on T-cell activation) (b) is consistent with the activation of cells. (c,d) contains a FACS histogram that shows the inhibition of PD-1 expression induced in the MLR by GSK-3 inhibitors, AR-A014418 (c) and CT99021 (d). Cell flow cytometry with an acquisition of 10 000 events and data analysis using FlowJo).

Another mode of T-cell activation involves the stimulation by the lectin Concanavalin A (Con A) (e, f). ConA binds α-D-mannosyl and α-D-glucosyl residues (two hexoses differing only by the alcohol on carbon 2) in terminal position of ramified structures from B-Glycans (reach in α-mannose, or hybrid and bi-antennary glycanes complexes). Con A is known to induce cell agglutination/clustering and to stimulate mouse T-cell subsets giving rise to four functionally distinct T cell populations, including precursors to suppressor T-cell (Dwyer and Johnson 1981 Clin Exp Immunol 46 (2): 237-49). (e) contains the bright field images of Con A activated cells with cell clustering observed in the presence and absence of GSK-3 inhibitor TDZD-8. Clustering was observed in Con A and Con A+TDZD-8 treated cells (arrows point to an example of a cluster). (f) contains the histogram of the percent of cells expressing PD-1 and shows that the non-ATP competitive GSK-3 inhibitor TDZD-8 inhibits PD-1 expression on Con A activated T-cells (>45% fewer PD-1 positive cells).

These data showed that the induction of PD-1 expression by other modes of stimulation that include the MLR and Con A is inhibited by the inhibition of GSK-3. Both ATP competitive and non-competitive inhibitors inhibited PD-1 expression induced by different modes of activation.

### Example 6: Anti-PD-1 Cooperates with GSK-3 Inhibition to Inhibit PD-1 Expression

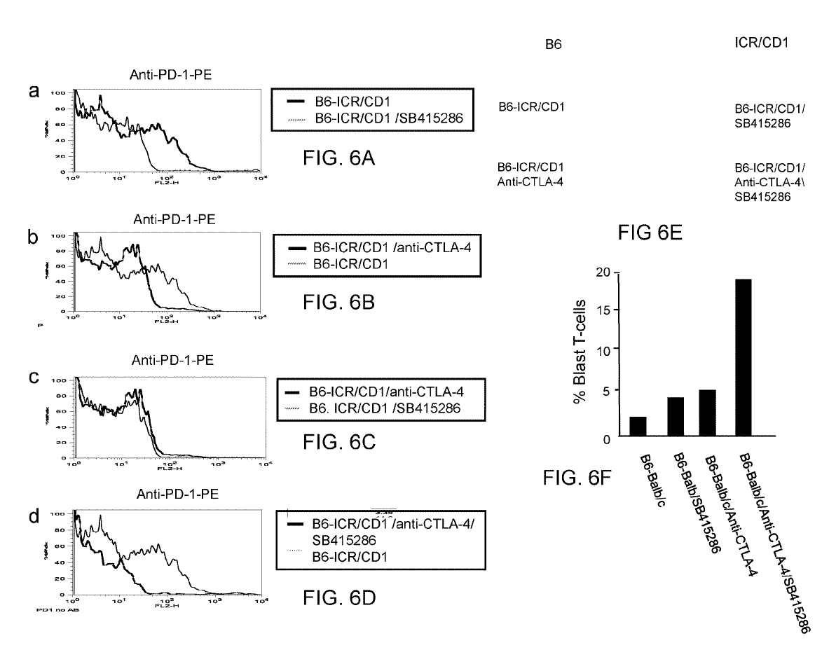

This example relates to the experiments in FIG. 6. FIG. 6 shows that anti-PD-1 ligation (clone RMP1-14 from Bio-XCell) cooperates with SB415286 inhibition of GSK-3 to inhibit PD-1 expression. (a) shows that SB415286 inhibits PD-1 expression (light line relative to dark line untreated control). (b) shows that anti-CTLA-4 also down-regulates the expression of PD-1 (dark line relative to light line untreated control); (c) shows that anti-CTLA-4 and S8415286 inhibited PD-1 expression to the same extent (dark and light lines); (d) contains a FACs profile that shows that the combined addition of anti-CTLA-4 and SB415286 inhibited PD-1 expression (dark line relative to light line untreated control) and to a greater extent than either anti-CTLA-4 or SB415286 alone (d versus c).

(e) shows the presence of cell clusters in B6/ICR/CD1 cultures in the absence and presence of anti-CTLA-4 and/or SB415286. (f) shows a histogram taken from the FACs analysis of forward scattered light (FSC) and side scattered light (SSC) which identified the presence of an activated population of larger blast T-cells (FSC is closely related to the size of cells-T-cell blasts being a larger population-FSC-H 150-200). This is a well-established procedure for identifying larger activated T-cell blasts. This figure shows that while the presence of anti-CTLA-4 and SB415286 increased the presence of blast T-cells, the combination of anti-CTLA-4 and SB415286 cooperated to synergistically increase the appearance of an activated blast population further indicative of enhanced T-cell activation.

These data show that anti-CTLA-4 can cooperate synergistically with GSK-3 inhibition by SB415286 to inhibit PD-1 expression and promote the appearance of T-cell blasts.

### Example 7: In Vivo Inhibition of GSK-3α/β by SB415286 Reduced PD-1 Transcription and Increased Tbet Transcription Concurrent with Elimination of EL4 Tumor Cells

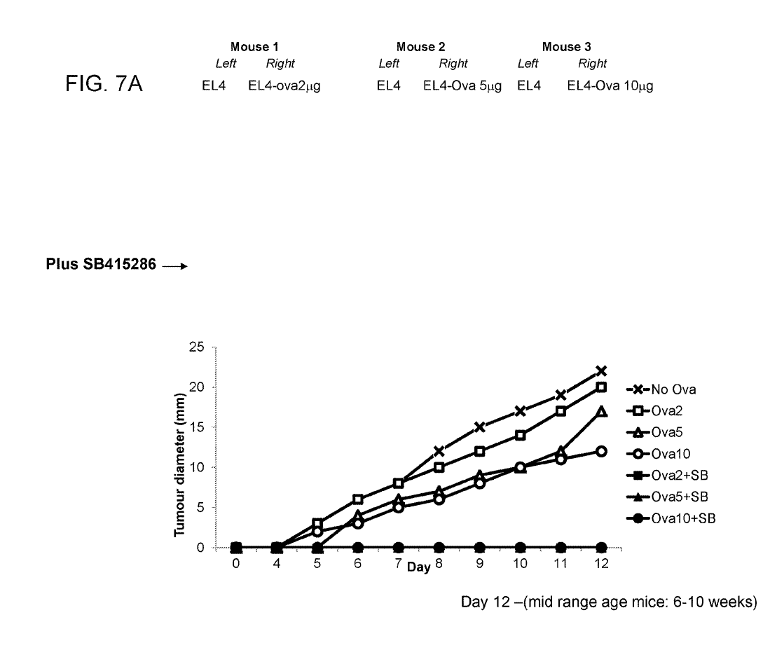

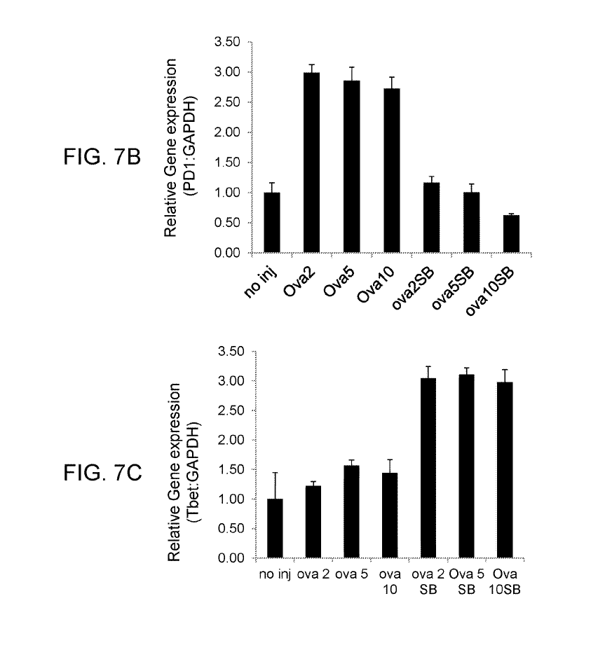

This example relates to the experiments in FIG. 7. Previous examples had shown that effect of GSK-3 inhibition on the expression of cells in vitro. This figure shows that the in vivo presence of SB415286 also inhibits PD-1 expression. For an in vivo cancer study, EL4 tumor cells taken from the log phase of growth were incubated in vitro with OVA peptide at different concentrations for 1 hr at 37° C. before washing with PBS and injecting into OT-1 Tg mice (typically 3×10⁶ cells in 50 ml) into the right flank skin. EL4 cells that had not incubated with OVA peptide were injected into the left flank as a control (3×10⁶ cells in 50 ml). In certain instances, EL-4 or EL-4-OVA cells were co-injected with SB215286. This is a well-established tumor model where the elimination of the tumor is dependent on the recognition of the EL4 tumor expressing the OVA peptide. Tumors grew progressively in an encapsulated fashion and were visible after 1 wk. Induced tumors were measured on a daily basis using a vernier caliper. Mice were mid-aged at 6-10 weeks old.

(a) shows images of tumors extracted from mice injected with EL4 cells (that had been pulsed with 2, 5 or 10 ug/of OVA peptide) in the presence of absence of S8415286. The presence of increasing amounts of OVA peptide resulted in small sized tumors (see upper panel: EL4-OVA-10 ug versus EL4-OVA-2 ug). The co-injection of SB415286 eliminated tumor growth at all OVA doses, 2 ug to 1 ug). Lower panel contains a histogram that shows tumor diameter over days 1-12 with a reduction in tumor size in response to increase concentrations of OVA in the absence of SB415286. In the presence of SB415286, tumors were completely eliminated at all OVA concentrations. The fact that tumor was visible when SB415286 was co-injected with EL4 cells in the absence of OVA peptide shows that the ability of GSK-3 inhibition to eliminate the tumors was dependent on an effect of SB415286 on the immune system reactivity against the tumor. These data shows that the in vivo injection of SB415286 promoted the elimination of tumors in response to

(b) contains a histogram that shows the qPCR measurements of PD-1 transcription from cells at day 12 where the presence of SB415286 in vivo inhibited PD-1 transcription. (c) contains a histogram that shows the qPCR measurements of Tbet transcription from cells at day 12 where the presence of SB415286 in vivo increased Tbet transcription.

The results in FIG. 7 demonstrate that the in vivo inhibition of GSK-3α/β with SB415286 reduced PD-1 transcription concurrent with increased Tbet transcription and the elimination of (EL4) tumor cells. It shows that the ability of SB415286 to eliminate tumors was dependent on its effect on immune system reactivity against the tumor.

### Example 8: In Vivo Inhibition of GSK-3α/β with SB415286 Reduced Tumor Growth to the Same Extent as Anti-PD-1 Therapy

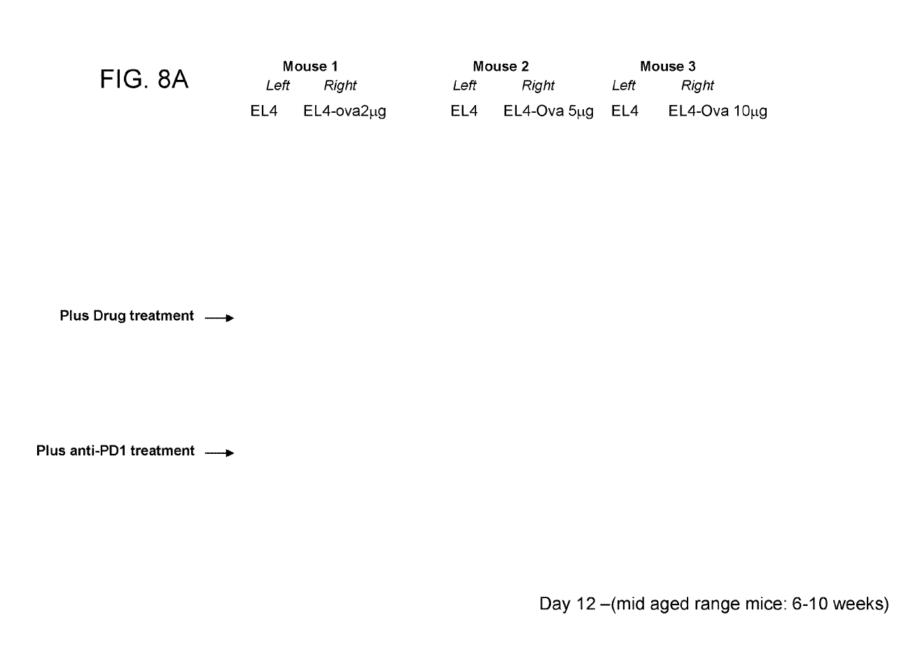

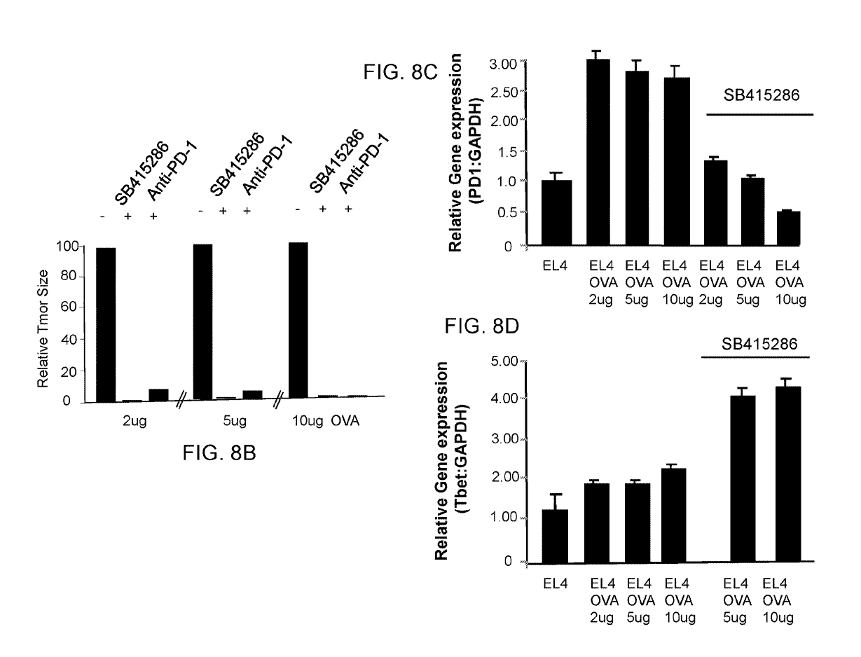

This example relates to the experiments in FIG. 8. These experiments were conducted as described in Example 7, except in certain instances, some mice were co-injected with tumor and anti-PD-1 antibody (clone RMP1-14 from Bio-XCell). Mice were mid-aged at 6-10 weeks old. Administration of SB415286 prevented tumor growth at all concentrations of OVA peptide (2, 5 and 10 ug) at 10 days (a). The same elimination to a lighter lessor degree was observed with anti-PD-1 treatment (a). (b) contains a histogram that shows a measurement of the tumor size relative to untreated control (i.e. 100%) in which SB415286 and anti-PD-1 markedly reduce tumor size at all OVA peptide doses. (c) is a histogram that shows the PCR measurements of PD-1 transcription from cells at day 12 showed an inhibition of PD-1 transcription at all doses of OVA peptide; (d) is a histogram that the qPCR measurements of Tbet transcription from cells at day 12 under the different conditions.

The results in FIG. 8 show that the in vivo administration of the GSK-3α/β inhibitor SB415286 reduced tumor size and PD-1 transcription to the same extent as anti-PD-1 alone. It also confirmed that in vivo administration of the GSK-3α/β inhibitor SB415286 increased Tbet transcription.

### Example 9: In Vivo Inhibition of GSK-3α/β with Another GSK-3 Inhibitor SB216763 Also Reduced PD-1 and Increased Tbet Transcription Concurrent with Elimination of EL4 Tumor Cells

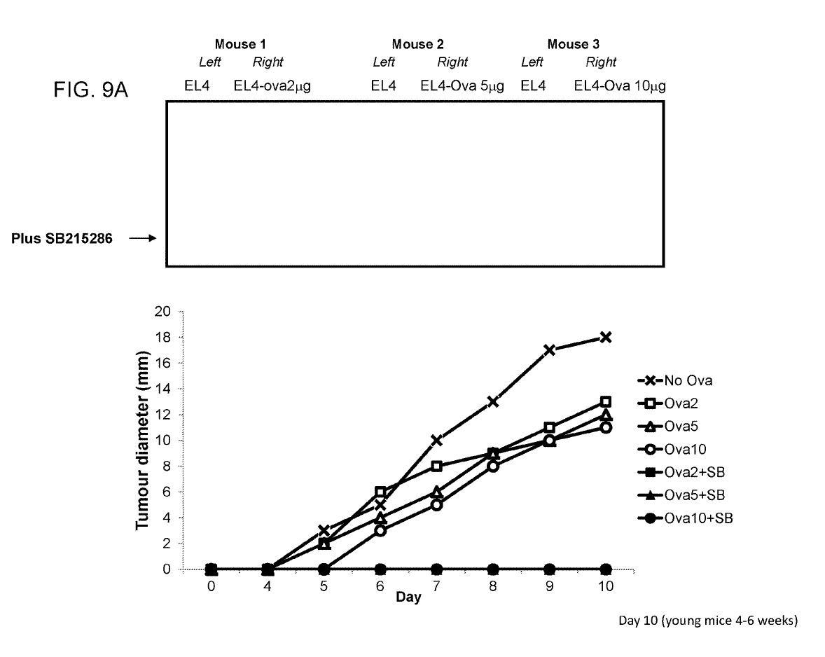

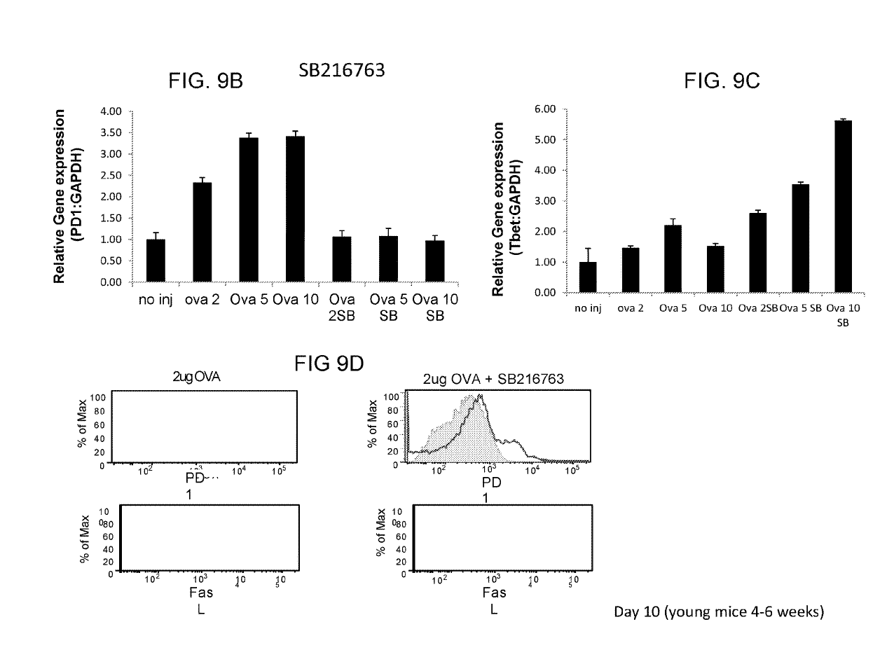

This example relates to the experiments in FIG. 9. In these experiments EL4 tumor cells were implanted and monitored as in Example 8 except that SB216763 was administered. Tumors were visible after 1 wk and grew progressively in an encapsulated fashion. Mice were young at 4-6 weeks. (a) shows that the administration of SB216763 prevented tumor growth at all concentrations of OVA peptide (2, 5 and 10 ug) over the full time course of 10 days (upper and lower panels). (b) contains a histogram of PCR measurements of PD-1 transcription from cells at day 12 which shows an inhibition of PD-1 transcription at all doses of OVA peptide; (c) contains a histogram of qPCR measurements of Tbet transcription from cells at day 12 which shows that SB216763 administration in vivo increased Tbet transcription. (d) shows by flow cytometry that PD-1 expression is reduced on T-cells from mice to which SB216763 was administered in vivo. The absence of an effect on FasL expression served as a negative control (d).

### Example 10: In Vivo Inhibition of GSK-3α/β with SB415286 Reduced PD-1 Transcription Concurrent with Elimination of EL4 Tumor Cells (6 Month Older Mice)

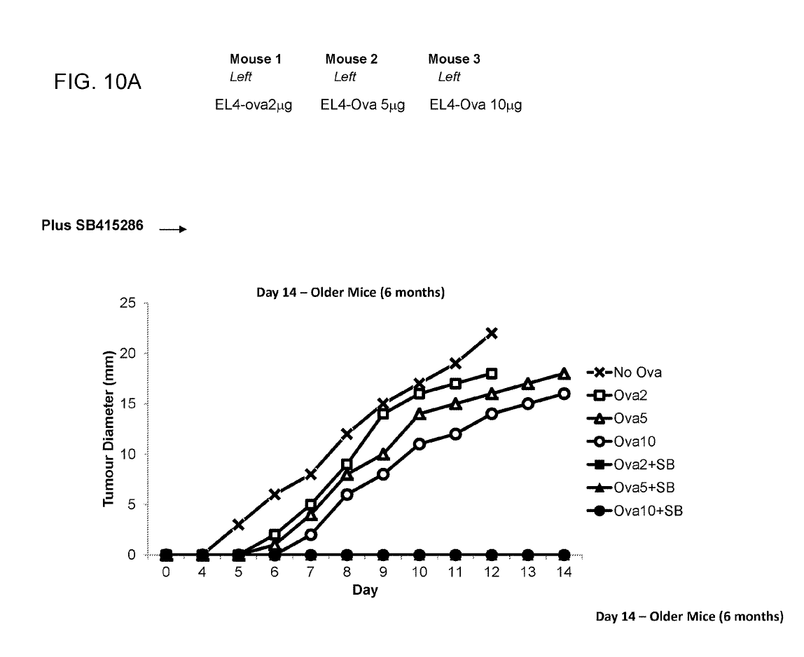

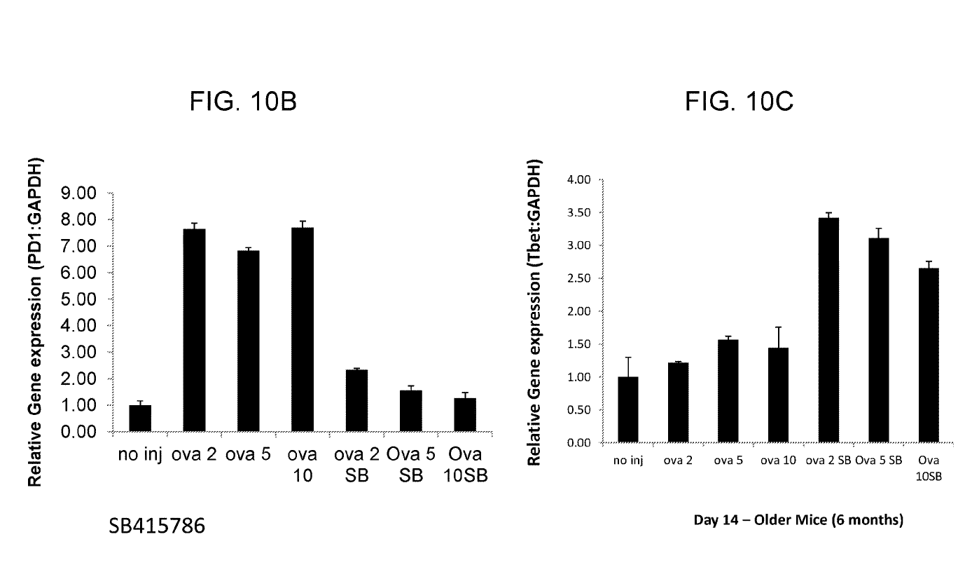

This example relates to the experiments in FIG. 10. In these experiments EL4 tumor cells taken from the log phase of in vitro growth and injected as outlined in Examples 7, 8 using SB415286. In the case, the mice were older at 6 months. Administration of SB415286 prevented tumor growth at all concentrations of OVA peptide (2, 5 and 10 ug) over the full time course of 10 days (a). (b) contains qPCR measurements of PD-1 transcription from cells at day 12 under the different conditions outlined in panel a. (c) contains qPCR measurements of Tbet transcription from cells at day 12 under the different conditions outlined in panel (a).

The results in FIG. 10 demonstrate that the GSK-3α/β inhibitor SB415286 reduced PD-1 transcription and increased Tbet expression concurrent with elimination of EL4 tumor cells in older mice.

### Example 11: Anti-PD-1 Cooperates with SB415286 to Reduce PD-1 Expression on T-Cells

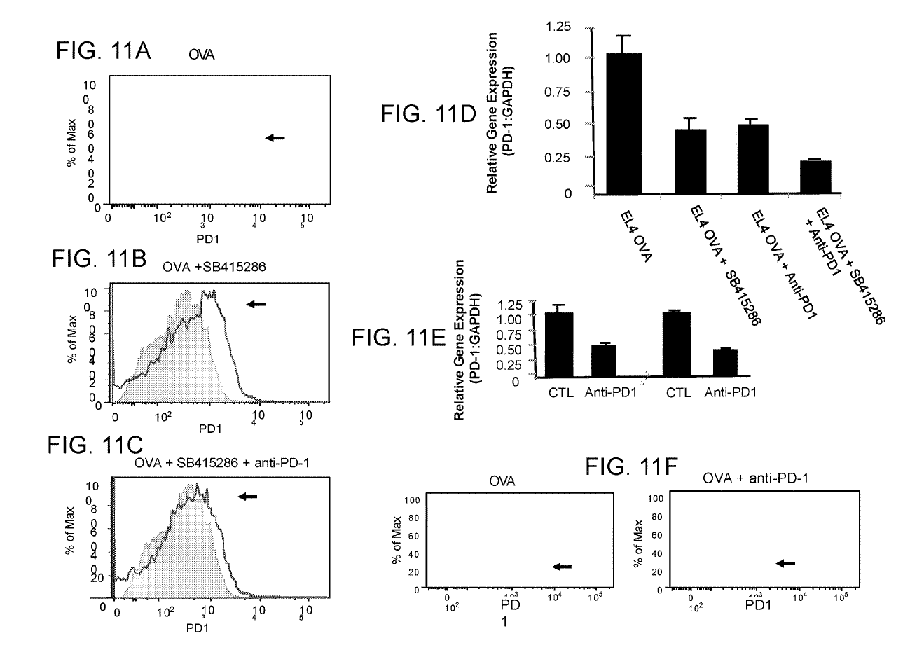

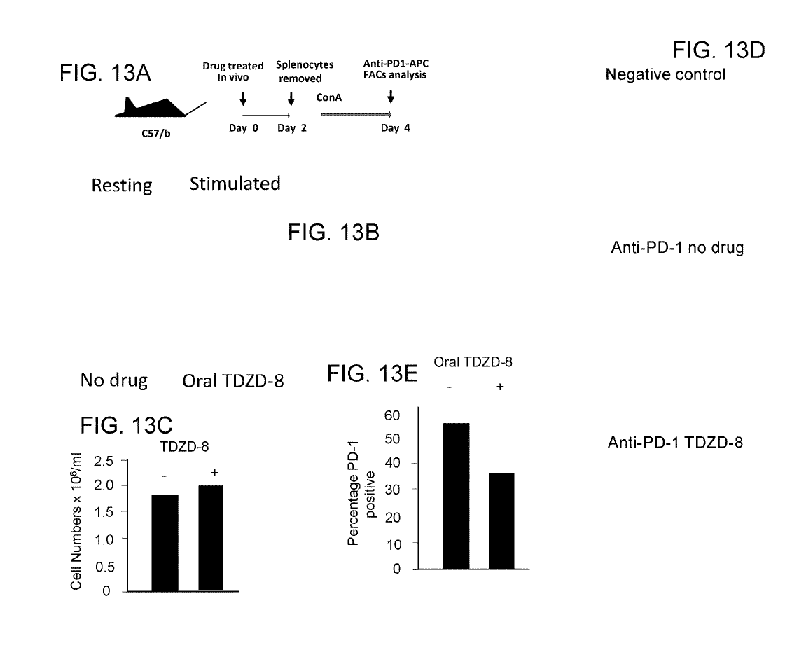

This example relates to the experiments in FIG. 11. In these experiments T-cells from the MHC class I-restricted OVA specific T cell receptor (TCR) transgenic mice (OT-1) mice with a TCR specific for the SIINFEKL peptide of OVAlbumin (OVA257-264) as presented by H-2kb were incubated in vitro with EL4-OVA peptide for 7 days. T-cells were incubated in the presence or absence of anti-PD-1 and then subjected to FACs or qPCR as described in the Materials and Methods. (a) contains a FACs profile that shows the expression of PD-1 on untreated cells (dark line versus background grey). (b) contains a FACs profile that shows incubation with SB415286 reduces PD-1 expression (dark line in b versus dark line in a). (c) contains a FACs profile that shows that the addition of anti-PD1 from the start of culture cooperates with SB415286 to further reduce the expression of PD-1. (d) shows qPCR values (relative gene expression-PD-1:GAPH) where SB415286 reduced PD-1 transcription from 1.0 to 0.45 and anti-PD-1 reduced transcription from 1.0 to 0.49. However, the combined exposure of cells to SB415286 and anti-PD-1 further decreased PD-1 transcription from 1.0 to 0.23. These data show that GSK-3 inhibition and anti-PD-1 can cooperate to decrease the expression of PD-1 on T-cells. The results in FIG. 13 provide evidence that GSK3 inhibitors will synergistically potentiate the effects of anti-PD-1 antibodies on PD-1 expression.

These data also make the important observation that anti-PD-1 ligation of cells can inhibit the transcription of PD-1. (e) shows the results of two experiments where anti-PD-1 inhibits the transcription of PD-1 by more than 50%. PD-1 can generate signals in T-cells due to it ligation that can reduce or turn off its own expression. (f) contains FACs profiles showing that anti-PD-1 ligation reduces the expression of PD-1 on cells.

### Example 12: In Vivo Inhibition of PD-1 with SB216763 is Accompanied by Increased Interferon-γ-1 Expression Under Conditions of Tumor Elimination

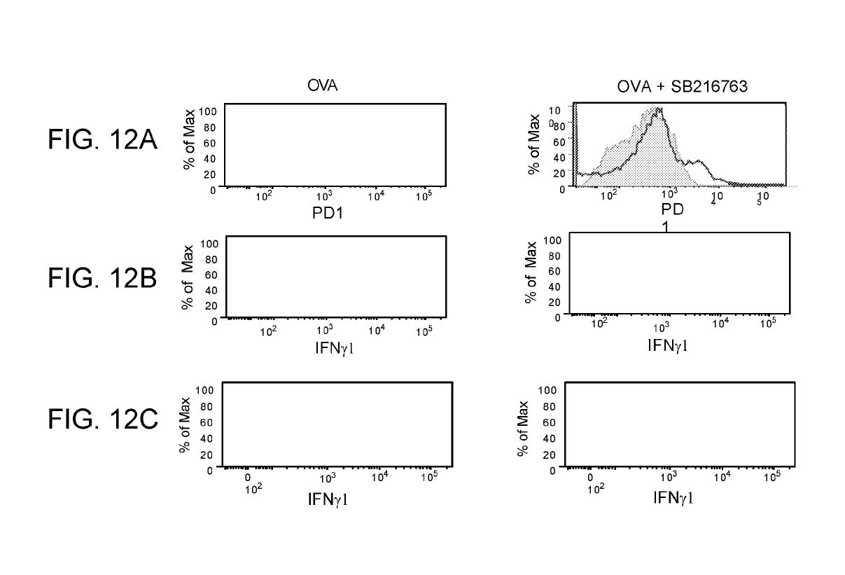

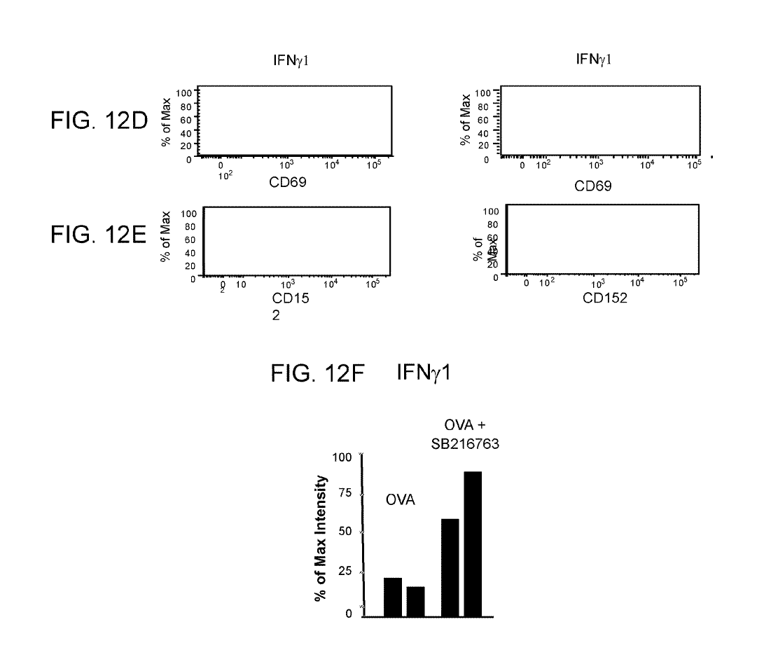

This example relates to the experiments in FIG. 12. In these experiments, EL4 tumor cells taken from the log phase of in vitro growth and injected as outlined in Examples 9 using SB415286. The co-injection of SB215763 down-regulated PD-1 and eliminated tumors. (a) shows that SB215763 reduced PD-1 expression. (b, c) contains FACs profiles showing that concurrent with reduced PD-1 is an increase in the percentage of cells that express IFNγ1. IFNγ inhibits viral replication directly, and is produced by CD4 helper and CD8 CTL effector cells once antigen-specific immunity develops (Schoenborn and Wilson (2007). Adv. Immunol. 96, 41-101). Mice homozygous for the Ifngr1^{tm1} targeted mutation are viable and normal T cell responses but are defective in natural resistance, evidenced by an increased susceptibility to infection by Listeria monocytogenes and vaccinia virus. Consistent with the increased activation state of the CTLs expected from the down-regulation of PD-1, there is an increase in CD69 (d) and a slight increase in the expression of CTLA-4 (CD152) (e). These observations are consistent with a Tbet/PD-1 driven augmentation of CTLs function that is expected for increased tumor elimination (as well as infections).

### Example 13: Oral Administration of GSK-3 Inhibitor In Vivo Inhibits PD-1 Expression

This example relates to the experiments in FIG. 13. B6 mice were fed water in a volume of 10 mls, either alone or in combination with TDZD-8 1 mg/ml as outlined in the Materials and Methods and as shown in (a). Ex vivo extracted cells were then cultured in 48 well tissue culture plates for 48 hours in the presence of anti-CD3 on plates (1 ug/ml). (b) shows the bright-field mages of T-cells in culture from the ocular. Equal numbers of cells were also observed in culture after 48 hours. (c) contains a histogram that shows equal numbers of cells in culture following ex vivo culturing of cells. There is no indication of cell death of cells in culture following the oral administration of TDZD-8. (d) contains the FACs profiles of anti-PD-1 staining (PE-Cy5) that shows a reduction in PD-1 expression on ex vivo cells from mice that had been given the drug TDZD-8 orally. Upper panel shows the negative control (i.e. no anti-PD-1 antibody) in staining. Middle panel shows the staining of cells with anti-PD-1, showing the expression of PD-1. Lower panel shows the reduced staining of cells with anti-PD-1 that had been administered the TDZD-8 drug orally (d). Cells from mice that had received TDZD-8 drug orally showed a lower expression of PD-1.

These data indicate the applicability of the oral administration of GSK-3 inhibitors for the down-regulation of PD-1 in the treatment of disease. It also indicates that the effect of TDZD-8 mediated PD-1 down-regulation can be maintained several days following the original in vivo exposure to the drug.

### Results

Initially, the effect of down-regulating or inhibiting GSK-3α/β was assessed on the expression of PD-1 in CD8+ T-cells from OT-1 TCR transgenic that were generated in vitro in response to the presentation of OVA peptide by EL4 cells (FIG. 1). T-cells from OVA specific T cell receptor (TCR) transgenic mice (OT-1) mice express a TCR that is specific for the SIINFEKL peptide of OVAlbumin (OVA257-264) as presented by H-2kb. Inhibition of GSK-3α/β with the inhibitors SB415286 or SB216763 over a period of 7 days reduced PD-1 surface expression (FIG. 1a,b). SB415286 and SB216763 are selective cell permeable and structurally distinct maleimides that inhibit GSK-3α with K(i)s of 31 nM and 9 nM respectively, in an ATP competitive manner. These compounds inhibited GSK-3β with similar potency. Neither compound significantly inhibited any member of a panel of 24 other protein kinases (Coghlan et al., 2000). Significantly, SB415286 decreased the number of cells expressing PD-1 from 30 to 7 percent, and a decrease in mean fluorescent intensity (MFI) from 13.6 to 4 (a). Similarly, SB216763 decreased the number of cells expressing PD-1 (b).

We next assessed the effects of GSK-3 on the transcription of PD-1 (FIG. 1c,d). SB415286 inhibited or arrested the induction of PD-1 transcription as determined by quantitative multiplex PCR (qPCR). Reverse transcription was performed using the RNA polymerase chain reaction (PCR) core kit followed by amplification for 40 cycles in a sequence detection system. mRNA expression was normalized against GAPDH expression using the standard curve method. While the presentation of OVA was normalized to a value of 1 for gene expression of PD-1 relative to control GAPDH, incubation with SB415286 reduced the value to 0.37, while SB216763 reduced the value to 0.04 (c). These data indicated for the first time that the inhibition of GSK3 by competitive inhibitors such as SB415286 or SB216763 markedly inhibit PD-1 transcription and expression.

At the same time, qPCR was run to assess the expression the transcription factor Tbet (Tbx21)(FIG. 1d). This was also measured in the context of OVA presentation of antigen by EL4 cells over 5 days in vitro in the presence or absence of SB415286. Concurrent with its inhibition of PD-1, SB415286 increased the expression of the transcription factor T-box transcription factor Tbet (Tbx21). The increase for relative gene expression (Tbet relative to the internal control GAPDH) increased from 1 for OVA alone to 2.5 for OVA plus SB415286 (d). This result indicated that the inhibition of GSK3 inhibited PD-1 transcription concurrent with the enhancement of Tbet transcription.

We next determined whether the GSK-3 reduction of PD-1 expression was itself responsible for enhanced CTL function by adding blocking anti-PD-1 or PD L1-Fc at the beginning of the culture between T-cells and EL4-OVA cells (FIG. 1e). The inhibition of expression and blocking PD-1 by the combination of both reagents increased CTL killing by 3-5 fold, and this increase matched the increase that was induced by SB415286. However, the addition of blocking anti-PD-1 or PDL1-Fc to cells expressing SB415286 did not increase killing beyond that seen with GSK-3α/β inactivation alone over the full range of CTL-target ratios (e). Identical results were obtained using the combination of SB216763 and anti-PD-1 or PDL1-Fc where the presence of anti-PD-1 or PDL1-Fc did not increase killing beyond that seen with SB216763 alone (FIG. 1f). This observation indicated that the GSK-3 modulatory effect on OT-1 CTL function was due to its down-modulation of PD-1 expression.

We next compared the effects of inhibiting GSK-3 on the expression of other receptors (FIG. 2). While SB415286 down-regulated the expression of PD-1 (FIG. 2a), it had not effect on the expression of CD3 (FIG. 2b) or CD44 (FIG. 2c). Similarly, incubation with SB216763 inhibited PD-1 expression (FIG. 3a), while having no effect on CD3 (FIG. 3b) or FasL (FIG. 3c). These data show that the inhibition of GSK3 inhibits preferentially the expression of PD-1.

We next assessed the effects of structurally distinct competitive and non-competitive inhibitors of GSK-3 on PD-1 expression (FIG. 4). Primary DO11.10 mouse T-cells were activated with either anti-CD3 (2C11) for 48 hours in the presence or absence of inhibitor followed by FACs analysis using anti-PD-1-PE. The figure shows that inhibition of PD-1 expression by each of the inhibitors tested that included SB216763, SB415286, L803-mts, AR-A014418, CT99021 and the thiadiazolidinone TDZD-8. Inhibition ranged from 40-60%. The chemical structures of each inhibitor are shown on bottom and right sides of figure. These data shows that despite the distinct nature of these chemicals, they shared the same ability to inhibit PD-1 expression as a result of sharing an ability to inhibit GSK-3.

We next assessed whether GSK-3 inhibitors could inhibit PD-1 expression in the context of a different mode of T-cell activation (FIG. 5). We therefore examined the effects of different GSK-3 inhibitors on PD-1 expression induced by a mixed lymphocyte reaction (MRL) (a-d) and Concanavalin A (Con A (e,f). Inbred C57BI/6 and outbred ICR/CD1 (Taconic labs) mouse spleen T-cells were either cultivated alone or co-cultured at equal numbers for 60 hours to induce an MLR, in the presence or absence of inhibitors AR-A014418 or CT99021 followed by FACs analysis for PD-1 expression. Splenocytes from outbred ICR/CD1 mice with a disparate MHC haplotype will mount a stronger immune response to inbred C57131/6 mice and vice versa. FIG. 5a shows the bright field images of B6 or ICR/CD1 T-cells alone or co-cultured in the absence or presence of AR-A014418. Clusters of cells are visible in the cultures containing mixed cells from different mice (lower panels), either with or without the GSK-3 inhibitor (arrow points to cell clusters). Resting monocultures shows a more diffuse distribution of cells. FACS analysis showed the inhibition of PD-1 expression on T-cells by AR-A014418 (c) and CT99021 (d). These data showed that GSK-4 inhibition by two different inhibitors can down-regulate PD-1 expression in the context of MPR stimulation.

Similarly, another GSK-3 inhibitor TDZD-8 was able to inhibit PD-1 expression in response to the lectin ConA (FIG. 5e). FIG. 5e shows the bright field images of resting versus ConA activated T-cells, in the presence or absence of inhibitor (arrow points to cell clusters). The inhibitor did not disrupt the ability of Con A to induced clusters. FIG. 5f shows the % of T-cells with PD-1 expression and the inhibition of expression by TDZD-8. These data showed that GSK-3 inhibition can inhibit PD-1 expression induced by a lectin Con A.

It was next of interest to determine whether GSK-3 inhibition could synergize with anti-CTLA-4 to down-regulate PD-1 expression. The rationale is the importance of combined therapies in the efficient amplification of the immune response against cancer and infectious diseases. FIG. 6 shows that GSK-3 inhibition by SB215286 cooperates with anti-CTLA-4 to down-regulate PD-1 and increase cell proliferation. C57BL/6J (B6) or outbred mouse CRI/CD1 T-cells were cultivated either alone or together at equal numbers (1×10⁶/ml) for 60 hours in the presence or absence of the inhibitor followed by the harvesting of cells and FACs analysis for PD-1 using anti-PD-1-PE. FIG. 6a shows that SB415286 reduced the expression of PD-1 on cells from B6/CRI/CD1 (C57BL/6J-CRI/CD1) cultures. Intriguingly, as shown in FIG. 6b, anti-CTLA-4 also reduced the expression of PD-1 when compared the B6/CRI/CD1 control. A comparison of the effects of either treatment showed that anti-CTLA-4 and SB415286 individually reduced the expression of PD-1 to a similar extent. However, as shown in FIG. 6d, the combination of anti-CTLA-4/SB415286 reduced the expression of PD-1 further (log scale), greater than each individually (compare to c). These data show that anti-CTLA-4 can synergize with GSK-3 inhibition to down-regulate PD-1 expression.

FIG. 6e shows the bright field images of cells cultured in the presence and absence of SB415286. As seen before, the MLR induced the appearance of activation clusters, both in the presence and absence of drug. However, as shown in FIG. 6f, anti-CTLA-4+SB415286 cooperated to increase the percent of T-cell blasts. The presence of blasts was determined by standard FSC gating that is related to the size of T-cell blasts. Activated T-cells are larger than resting T-cells. Consistent with the promotion of activation and effectors, the presence of SB415286 increased the size of the blast population by some 4-5 fold.

We next assessed whether the inhibition of GSK-3 could also inhibit PD-1 expression in vivo in the context of the recognition and elimination of tumor cells in 6-10 week old mice (FIG. 7). EL4 tumor cells were taken from the log phase of in vitro growth and pulsed with OVA peptide for 1 hr at 37 C before washing and injecting into young OT-1 Tg mice (typically 3×10⁶ cells). EL4 cells were co-injected with/without SB415286 into the right flank skin and non-pulsed EL4 cells were injected into the left flank to act as a control. Tumors were clearly visible after 1 week and grew progressively in an encapsulated fashion. Induced tumors were measured on a daily basis using a vernier caliper. Tumors and spleens were harvested on day 10 when PCR was performed. As shown in 3 mice, the injection of EL4 tumor cells resulted in the growth of the tumor as seen at day 12 that was reduced by the injection of OVA peptide at 2, 5 and 10 ug, relative to the PBS control as evident at days 7 to 10. By contrast, the co-injection of SB415286 completely prevented the growth of the tumor in the presence of OVA peptide (upper panels and lower histogram).

qPCR of PD-1 expression also showed that the transcription of PD-1 increased with OVA peptide from 2 to 5-10 ug (FIG. 7b). By contrast, the co-incubation with SB415286 prevented to increase in expression in the presence of 2, 5 and 10 ug OVA peptide. The level of PD-1 transcription in the presence of OVA plus SB415286 was the same as the level of PD-1 in the absence of tumor. The same experiment showed an increase in the transcription of Tbet in the presence of SB415286 (FIG. 7c).

Similar effects were seen in a separate experiment that included the inclusion of anti-PD-1 during injection of EL-4-OVA in mice aged 6-10 weeks (FIG. 8a). SB415286 prevented tumor growth to a similar extent (or a slightly greater extent) than seen with the anti-PD-1 blockade. Neither had a consistent effect on the size of EL4 tumor masses lacking OVA peptide. FIG. 8b shows a comparison of the effects of SB415286 and anti-PD-1 where both dramatically reduced the size of the tumors. Further, while the injection of 2, 5 and 10 ug/ml of OVA peptide induced an increase in the transcription of injection of PD-1, the inhibition of GSK3 by co-injection of SB415286 arrested the increase in transcription of PD-1 (FIG. 8c). Concurrently, the presence of SB415286 also increased the transcription of the transcription of Tbet (FIG. 8d). Overall, these data show that the co-incubation of EL4-OVA with SB415286 concurrently inhibited PD-1 transcription while enhancing Tbet transcription.

Similar results were obtained using younger mice from 4-6 weeks old using SB216763 (FIG. 9a). T-cells were isolated from mice that had been co-injected with EL4 tumor, OVA peptide and the other inhibitor SB216763 tumors and spleens were harvested on day 10 when PCR was performed. As shown in 3 mice, the injection of EL4 tumor cells resulted in the growth of the tumor that was reduced by the injection of Ova peptide at 2, 5 and 10 ug relative to the PBS control as evident at days 8 to 10. By contrast, the co-injection of SB216763 completely prevented the seeding and growth of the tumor in the presence of Ova peptide (see upper panels and lower histogram). qPCR on samples showed that SB216763 prevented to increase in PD-1 transcription in the presence of 2, 5 and 10 ug OVA peptide (FIG. 9b) while showing an increase for Tbet transcription (FIG. 9c). FACs analysis confirmed that the in vivo administration of the drug inhibited PD-1 expression but not FasL expression (FIG. 9d).

Similar results were obtained with older mice at 6 months where the injection of SB415286 prevented completely the growth of tumors (FIG. 10a) and similarly a reduction in PD-1 transcription (FIG. 10b) while showing an increase for Tbet transcription (FIG. 10c).

Given the demonstration that anti-CTLA-4 can synergize with GSK-3 inhibition to down-regulate PD-1 expression, we next assessed whether anti-PD-1 could also cooperate with SB415286 (FIG. 11). Figures a-c show that anti-PD-1 cooperates with SB415286 inhibition of GSK-3 to down-regulate the expression of PD-1 on the surface of T-cells. FIG. 11a shows the expression of PD-1 on OT-1 T-cells stimulated by EL-4-OVA presentation to OT-1 T-cells in vitro, which was down regulated by the presence of SB415286 from the start of culture (b). Intriguingly, anti-PD-1 cooperated with SB415286 to reduce PD-1 expression further on OVA activated OT 1 T-cells (see relative to b). These data showed anti-PD-1 can cooperate with GSK-3 inhibition to inhibit the expression of PD-1 on the surface of T-cells.

To determine more about the mechanism of action of anti-PD-1, quantitative PCR analysis was conducted and showed that anti-PD-1 ligation of cells can itself also inhibit PD-1 transcription (FIG. 11d). Further, SB415286 synergized with anti-PD-1 to maximally inhibit PD-1 transcription. FIG. 11e shows further examples of anti-PD-1 inhibition of its own transcription on T-cells (two additional experiments). Fig. f shows the down-regulation of PD-1 due to anti-PD-1 ligation as seen by FACs staining with anti-PD-1-PE. The results show that PD-1 expression and transcription is inhibited by the GSK-3 inhibitors and by the anti-PD-1 antibody and importantly, they cooperate to maximally suppress PD-1 transcription.

PD-1 is known to function as a negative regulator of T-cell function. The blockade of PD-1 in turn facilitates greater T-cell functionality and CTL function. Our finding that GSK-3 inhibition could increase CTL function and the elimination of tumors suggested that it would increase T-cell functionality. One aspect of CTL functionality on CD8+ T-cells is the expression of Interferon-γ1, (IFN-γ1). We therefore next assess whether SB216763 could increase the expression of IFN-γ1. FIG. 12 shows that the drug induced in vivo down-regulation of PD-1 and tumor elimination was accompanied by an increase in the expression of IFN-γ1. FIG. 12a shows the down-regulation of PD-1 by SB216763, while (b) and (c) show an increase expression of IFN-γ1 on a greater number of cells. There was also a minor increase in CD69 expression indicative of greater T-cell activation as well as CTLA-4, an activation antigen on T-cells. Figure f shows a histogram representation of the % max intensity of IFN-γ1 due to PD-1 down-regulation and GSK-3 inhibition.

Another key question was whether GSK-3 inhibitors could be administered orally to achieve the inhibition of PD-1 expression. This would allow the drug to be taken as a tablet in the treatment of cancer or infectious diseases. FIG. 13 shows that the oral administration in vivo inhibits PD-1 expression. FIG. 13a shows a histogram showing the regime of oral drug administration. Mice were feed TDZD-8 orally in the water. FIG. 13b shows the bright-field mages of T-cells in culture from the ocular, while (b) shows that equal numbers of cells were observed in culture following ex vivo culturing of cells. The drug therefore had no obvious long-term effect on viability. FIG. 113d shows the FACs profiles of ex vivo cultured and activated cells taken from mice that had been treated with no drug (middle panel) or the drug (lower panel). a reduction in PD-1 expression on ex vivo cells from mice that had been given the drug TDZD-8 orally. FIG. 13e presents a histogram showing that the in vivo oral administration of TDZD-8 reduce the percentage of T-cells expressing PD-1. These data show that an inhibitor of GSK-3 can be administered orally to achieve the down-regulation of PD-1.

### CONCLUSIONS

The data in the foregoing experiments provides convincing evidence that GSK-3 inhibitors specifically inhibit or arrest the transcription and expression of PD-1 by T cells, and promote Tbet expression and further demonstrate that the inhibition or arrest of PD-1 expression promotes T cell immunity, and in particular promotes anti-tumor immunity. GSK-3 inhibits of distinct chemical structure (ATP competitive and non-competitive inhibitors) showed the same ability to down-regulate PD-1 expression. Further, we showed that the oral in vivo uptake of a GSK-3 inhibitor can inhibit PD-1 expression when induced by subsequent in vitro activation. Further, GSK-3 inhibitors could down regulate PD-1 on T-cells activated by a variety of means, including anti-CD3 ligation, antigen presentation (i.e. OVA peptide), the mixed lymphocyte reaction and by the lectin Con A. This observation strongly suggests that GSK-3 inhibitors can be applied in the range of different situations and stimuli involved in the activation of T-cell responses. Based on this observation, various GSK inhibitors may be used to promote CTL immunity and to treat conditions wherein the suppression of PD-1 expression and/or enhanced CTL immunity is therapeutically desired such as cancer and infectious disease conditions.

This is an exciting discovery as despite its central importance in programmed death 1 (PD-1), the proximal signalling pathway that couples the T-cell receptor to PD-1 expression was undefined. The experiments herein demonstrate that glycogen synthase kinase 3α/β is the central upstream signalling node that controls PD-1 transcription, and that GSK3 inhibitors suppress the transcription and surface expression of PD-1. Therefor such GSK-3 inhibitors may be used to inhibit the suppressive effects of PD-1 on T cell immunity and thereby promote CTL immune responses.

The findings also make the important observation that both anti-CTLA and anti-PD-1 synergize with GSK-3 inhibitors to down-regulate PD-1 expression. Also, we found anti-CTLA-4 synergized with SB415286 to reduce PD-1 expression and increase the number of T-cell blasts induced in the MLR. Further, anti-PD-1 ligation was found to inhibit the transcription of PD-1 to the same extent as GSK-3 inhibition. Anti-PD-1 and SB415286 cooperated to increase further PD-1 inhibition.

These data indicate that these inhibitors may be used alone; however, advantageously such GSK-3 inhibitors will be combined with other immune potentiators, especially those that promote T cell immunity. Examples thereof include anti-PD-1, anti-PD-L1, anti-CTLA4, anti-TIMP, CD40 agonists, TLR agonists, 4-1BB agonists, CD27 agonists and the like.

### THERAPEUTIC APPLICATIONS

As noted, the subject GSK-3 modulators may be used to treat different conditions wherein upregulation of T cell immunity is therapeutically desired such as cancer or infectious conditions such as carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD).

Also, cancers amenable for treatment using the GSK-3 modulatory compounds of the present invention include, but not limited to, carcinoma, lymphoma, blastoma, sarcoma, and leukemia or lymphoid malignancies. More particular examples of such cancers include bladder, ovarian, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancer may be an early advanced (including metastatic) bladder, ovarian or melanoma. The cancer may be colorectal cancer. The cancerous conditions amenable for treatment of the invention include metastatic cancers and the treatment of vascularized tumors.

Further the subject GSK-3 modulators, e.g., GSK-3 inhibitors, may be used to treat infectious conditions, e.g., viral, bacterial, fungal or parasitic infectious conditions. Examples thereof include e.g., hepatitis B, hepatitis C, Epstein-Barr virus, cytomegalovirus, immunodeficiency virus (HIV) infection, HIV-1, HIV-2, herpes, papillomavirus infection and associated diseases, tuberculosis, malaria, schistosomiasis. echovirus infection, parvovirus infection, rubella virus infection, post-vaccination syndromes, congenital rubella infection, pertussis, influenza, mumps, and Epstein-Barr virus-associated diseases.

Also, as further noted, the subject GSK-3 modulators, e.g., GSK-3 activators, may be used to treat different conditions wherein downregulation of T cell immunity is therapeutically desired such as autoimmunity, allergy or inflammatory conditions such as afore-mentioned. The subject GSK-3 modulators may be administered systemically or locally depending on the nature of the compound and disease condition treated. This includes administration by oral route, inhalation, injection (intravenous, subcutaneous, intramuscular . . . ,), topical, suppository, and other known routes of administration.

The subject GSK-3 modulators, i.e., inhibitors or activators, may be used alone or in association with other therapeutic agents wherein such therapeutic agents may include other biologics or non-biologics such as small molecules, chemotherapeutics, anti-infectives, anti-inflammatory agents, anti-allergenic agents, radionuclides, other receptor agonists or antagonists, hormone modulators, growth factor modulators and the like. Suitable therapeutics for treating cancer, infectious diseases, inflammatory, allergic or autoimmune conditions are known in the art. The selection of appropriate other therapeutic agent will depend on the specific condition being treated.

The subject GSK3 modulators, i.e., inhibitors or activators, of the invention when used for therapy will be incorporated into pharmaceutical compositions suitable for therapeutic administration. Such compositions will typically comprise an effective amount of the compound and a carrier, e.g., a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Toxicity and therapeutic efficacy of such compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of drug will vary dependent on the particular GSK-3 modulator (i.e., an effective dosage). For example, it may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a GSK-3 modulator can include a single treatment or, more typically will include a series of treatments.

The administration of GSK-3 modulators, i.e., GSK-3 inhibitors or activators, according to the invention may be through various routes, for example oral, rectal, nasal, pulmonary, topical (including Buccal and sublingual), transdermal, intraperitoneal, vaginal, parenteral (including subcutaneous, intramuscular, intradermal), intrathecal or intracerebroventricular. It will be appreciated that the preferred route will depend on the general condition and age of the subject to be treated, the nature of the condition to be treated and the active ingredient chosen.

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a formulation which may be a solution or suspension for the administration of the GSK-3 inhibitors in the form of a nasal or pulmonal spray. As a still further option, the formulation containing the GSK-3 inhibitor of the invention can also be adapted to transdermal administration, e.g. by needle-free injection or from a patch, optionally an iontophoretic patch, or transmucosal, e.g. buccal, administration.

GSK-3 modulators, i.e., GSK-3 inhibitors or activators, of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatin capsules and soft gelatin capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

GSK-3 modulators, i.e., GSK-3 inhibitors or activators, of the invention may further be compounded in, or attached to, for example through covalent, hydrophobic and electrostatic interactions, a drug carrier, drug delivery system and advanced drug delivery system in order to further enhance stability of the composition, increase bioavailability, increase solubility, decrease adverse effects, achieve chronotherapy well known to those skilled in the art, and increase patient compliance or any combination thereof.

Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behavior in lipid-water systems, polymeric micelles, multiple emulsions, self-emulsifying, self-microemulsifying, cyclodextrins and derivatives thereof, and dendrimers.

GSK-3 modulators, i.e., inhibitors or activators, of the current invention may be useful in the composition of solids, semi-solids, powder and solutions for pulmonary administration, using, for example a metered dose inhaler, dry powder inhaler and a nebulizer, all being devices well known to those skilled in the art.

GSK-3 inhibitors of the current invention may be useful in the composition of controlled, sustained, protracting, retarded, and slow release drug delivery systems. More specifically, but not limited to, modulators are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles.

Methods to produce controlled release systems useful for compositions of the current invention include, but are not limited to, crystallization, condensation, co-crystallization, precipitation, co-precipitation, emulsification, dispersion, high pressure homogenization, en-capsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (McNally, E. J., ed. Marcel Dekker, New York, 2000).

The dose of a GSK-3 modulators, i.e., inhibitors or activators, according to the present invention may also be administered prior to the onset of infection or reoccurrence as in herpes or other viruses that are subject to reoccurrence of infection or autoimmune, allergic or inflammatory conditions subject to repeated or chronic reoccurrence or flare-up of autoimmunity, allergy or inflammation.

### REFERENCES

1. Agata, Y., Kawasaki, A., Nishimura, H., Ishida, Y., Tsubata, T., Yagita, H., and Honjo, T. (1996). Expression of the PD-1 antigen on the surface of stimulated mouse T and B lymphocytes. International immunology 8, 765-772.
2. Ahmed, R., Bevan, M. J., Reiner, S. L., and Fearon, D. T. (2009). The precursors of memory: models and controversies. Nat Rev Immunol 9, 662-668.
3. Ali, A., Hoeflich, K. P., and Woodgett, J. R. (2001). Glycogen synthase kinase-3: properties, functions, and regulation. Chemical reviews 101, 2527-2540.
4. Atayar, C., Poppema, S., Blokzijl, T., Harms, G., Boot, M., and van den Berg, A. (2005). Expression of the T-cell transcription factors, GATA-3 and T-bet, in the neoplastic cells of Hodgkin lymphomas. Am J Pathol 166, 127-134.
5. Beals, C. R., Sheridan, C. M., Turck, C. W., Gardner, P., and Crabtree, G. R. (1997). Nuclear export of NF-ATc enhanced by glycogen synthase kinase-3. Science 275, 1930-1934.
6. Chung, H. T., Kim, L. H., Park, B. L., Lee, J. H., Park, H. S., Choi, B. W., Hong, S. J., Chae, S. C., Kim, J. J., Park, C. S., and Shin, H. D. (2003). Association analysis of novel TBX21 variants with asthma phenotypes. Hum Mutat 22, 257.
7. Coghlan, M. P., Culbert, A. A., Cross, D. A., Corcoran, S. L., Yates, J. W., Pearce, N. J., Rausch, 01., Murphy, G. J., Carter, P. S., Roxbee Cox, L., et al. (2000). Selective small molecule inhibitors of glycogen synthase kinase-3 modulate glycogen metabolism and gene transcription. Chemistry & biology 7, 793-803.
8. Cohen, P., and Frame, S. (2001). The renaissance of GSK3. Nat Rev Mol Cell Biol 2, 769-776.
9. Crabtree, G. R., and Olson, E. N. (2002). NFAT signaling: choreographing the social lives of cells. Cell 109 Suppl, S67-79.
10. Day, C. L., Kaufmann, D. E., Kiepiela, P., Brown, J. A., Moodley, E. S., Reddy, S., Mackey, E. W., Miller, J. D., Leslie, A. J., DePierres, C., et al. (2006). PD-1 expression on HIV-specific T cells is associated with T-cell exhaustion and disease progression. Nature 443, 350-354.
11. Desjeux, P. (2001). The increase of risk factors for leishmaniasis worldwide. Transactions of the Royal Society of Tropical Medicine and Hygiene 95, 239-243.
12. Dikici, B., Kalayci, A. G., Ozgenc, F., Bosnak, M., Davutoglu, M., Ece, A., Ozkan, T., Ozeke, T., Yagci, R. V., and Haspolat, K. (2003). Therapeutic vaccination in the immunotolerant phase of children with chronic hepatitis B infection. Pediatr. Infect. Dis. J. 22, 345-349.
13. Dorfman, D. M., Hwang, E. S., Shahsafaei, A., and Glimcher, L. H. (2005). T-bet, a T cell-associated transcription factor, is expressed in Hodgkin's lymphoma. Hum Pathol 36, 10-15.
14. Factbook, C.-T. W. (2007). “Central Intelligence Agency, 4 “cia.gov”.
15. Frame, S., and Cohen, P. (2001). GSK3 takes centre stage more than 20 years after its discovery. Biochem J 359, 1-16.
16. Freeman, G. J., Long, A. J., Iwai, Y., Bourque, K., Chernova, T., Nishimura, H., Fitz, L. J., Malenkovich, N., Okazaki, T., Byrne, M. C., et al. (2000). Engagement of the PD-1 immunoinhibitory receptor by a novel B7 family member leads to negative regulation of lymphocyte activation. J Exp Med 192, 1027-1034.
17. Freeman, G. J., Wherry, E. J., Ahmed, R., and Sharpe, A. H. (2006). Reinvigorating exhausted HIV-specific T cells via PD-1-PD-1 ligand blockade. J Exp Med 203, 2223-2227.
18. Greenwald, R. J., Freeman, G. J., and Sharpe, A. H. (2005). The B7 family revisited. Annu Rev Immunol 23, 515-548.
19. Hooper, C., Killick, R., and Lovestone, S. (2008). The GSK3 hypothesis of Alzheimer's disease. J Neurochem 104, 1433-1439.
20. Horne-Debets, J. M., Faleiro, R., Karunarathne, D. S., Liu, X. Q., Lineburg, K. E., Poh, C. M., Grotenbreg, G. M., Hill, G. R., Macdonald, K. P., Good, M. F., et al. (2013). PD-1 Dependent Exhaustion of CD8(+) T Cells Drives Chronic Malaria. Cell reports 5, 1204-1213.
21. Intlekofer, A. M., Takemoto, N., Wherry, E. J., Longworth, S. A., Northrup, J. T., Palanivel, V. R., Mullen, A. C., Gasink, C. R., Kaech, S. M., Miller, J. D., et al. (2005). Effector and memory CD8+ T cell fate coupled by T-bet and eomesodermin. Nat Immunol 6, 1236-1244.
22. Ishida, Y., Agata, Y., Shibahara, K., and Honjo, T. (1992). Induced expression of PD-1, a novel member of the immunoglobulin gene superfamily, upon programmed cell death. Embo J 11, 3887-3895.
23. James, W. D., Timothy, G., and al., e. (2006). Andrews' Diseases of the Skin: clinical Dermatology. Saunders Elsevier. ISBN 0-7216-2921-0.
24. Jope, R. S., and Roh, M. S. (2006). Glycogen synthase kinase-3 (GSK3) in psychiatric diseases and therapeutic interventions. Curr Drug Targets 7, 1421-1434.
25. Juedes, A. E., Rodrigo, E., Togher, L., Glimcher, L. H., and von Herrath, M. G. (2004). T-bet controls autoaggressive CD8 lymphocyte responses in type 1 diabetes. J Exp Med 199, 1153-1162.
26. Kamphorst, A. O., and Ahmed, R. (2013). Manipulating the PD-1 pathway to improve immunity. Curr Opin Immunol 25, 381-388.
27. Kao, C., Oestreich, K. J., Paley, M. A., Crawford, A., Angelosanto, J. M., Ali, M. A., Intlekofer, A. M., Boss, J. M., Reiner, S. L., Weinmann, A. S., and Wherry, E. J. (2011). Transcription factor T-bet represses expression of the inhibitory receptor PD-1 and sustains virus-specific CD8+ T cell responses during chronic infection. Nat Immunol 12, 663-671.
28. Keir, M. E., Butte, M. J., Freeman, G. J., and Sharpe, A. H. (2008). PD-1 and its ligands in tolerance and immunity. Annu Rev Immunol 26, 677-704.
29. Kinter, A. L., Godbout, E. J., McNally, J. P., Sereti, I., Roby, G. A., O'Shea, M. A., and Fauci, A. S. (2008). The common gamma-chain cytokines IL-2, IL-7, IL-15, and IL-21 induce the expression of programmed death-1 and its ligands. J Immunol 181, 6738-6746.
30. Klenerman, P., and Hill, A. (2005). T cells and viral persistence: lessons from diverse infections. Nat. Immunol. 6, 873-879.
31. Kulkarni, A., Ravi, D. S., Singh, K., Rampalli, S., Parekh, V., Mitra, D., and Chattopadhyay, S. (2005). HIV-1 Tat modulates T-bet expression and induces Th1 type of immune response. Biochem Biophys Res Commun 329, 706-712.
32. Kuo, G. H., Prouty, C., DeAngelis, A., Shen, L., O'Neill, D. J., Shah, C., Connolly, P. J., Murray, W. V., Conway, B. R., Cheung, P., et al. (2003). Synthesis and discovery of macrocyclic polyoxygenated bis-7-azaindolylmaleimides as a novel series of potent and highly selective glycogen synthase kinase-3β inhibitors. J Med Chem 46, 4021-4031.
33. Macian, F. (2005). NFAT proteins: key regulators of T-cell development and function. Nat Rev Immunol 5, 472-484.
34. Maini, M. K., Boni, C., Ogg, G. S., King, A. S., Reignat, S., Lee, C. K., Larrubia, J. R., Webster, G. J., McMichael, A. J., Ferrari, C., and al., e. (1999). Direct ex vivo analysis of hepatitis B virus-specific CD8(+) T cells associated with the control of infection. Gastroenterology. 117, 1386-1396.
35. Mazanetz, M. P., and Fischer, P. M. (2007). Untangling tau hyperphosphorylation in drug design for neurodegenerative diseases. Nat Rev Drug Discov 6, 464-479.
36. Morrison, W. I., Murray, M., Whitelaw, D. D., and Sayer, P. D. (1983). Pathology of infection with Trypanosoma brucei: disease syndromes in dogs and cattle resulting from severe tissue damage. Contributions to microbiology and immunology 7, 103-119.
37. Murray, M., Morrison, W. I., and Whitelaw, D. D. (1982). Host susceptibility to African trypanosomiasis: trypanotolerance. Advances in parasitology 21, 1-68.
38. Neal, J. W., and Clipstone, N. A. (2001). Glycogen synthase kinase-3 inhibits the DNA binding activity of NFATc. J Biol Chem, 3666-3673.
39. Nisii, C., Tempestilli, M., Agrati, C., Poccia, F., Tocci, G., Longo, M. A., D'Offizi, G., Tersigni, R., Lo Iacono, O., Antonucci, G., and Oliva, A. (2006.). Accumulation of dysfunctional effector CD8+ T cells in the liver of patients with chronic HCV infection. J. Hepatol. 44, 475-483.
40. Oestreich, K. J., Yoon, H., Ahmed, R., and Boss, J. M. (2008). NFATc1 regulates PD-1 expression upon T cell activation. J Immunol 181, 4832-4839.
41. Ohteki, T., Parsons, M., Zakarian, A., Jones, R. G., Nguyen, L. T., Woodgett, J. R., and Ohashi, P. S. (2000). Negative regulation of T cell proliferation and interleukin 2 production by the serine threonine kinase GSK-3. J Exp Med 192, 99-104.
42. Raby, B. A., Hwang, E. S., Van Steen, K., Tantisira, K., Peng, S., Litonjua, A., Lazarus, R., Giallourakis, C., Rioux, J. D., Sparrow, D., et al. (2006). T-bet polymorphisms are associated with asthma and airway hyperresponsiveness. Am J Respir Crit Care Med 173, 64-70.
43. Rao, A., Luo, C., and Hogan, P. G. (1997). Transcription factors of the NFAT family: regulation and function. Annu Rev Immunol 15, 707-747.
44. Rovedo, M. A., Krett, N. L., and Rosen, S. T. (2011). Inhibition of glycogen synthase kinase-3 increases the cytotoxicity of enzastaurin. J Invest Dermatol 131, 1442-1449.
45. Rudd, C. E. (1999). Adaptors and molecular scaffolds in immune cell signaling. Cell 96, 5-8.
46. Rudd, C. E., and Schneider, H. (2003). Unifying concepts in CD28, ICOS and CTLA4 co-receptor signalling. Nat Rev Immunol 3, 544-556.
47. Sakuishi, K., Apetoh, L., Sullivan, J. M., Blazar, B. R., Kuchroo, V. K., and Anderson, A. C. (2010). Targeting Tim-3 and PD-1 pathways to reverse T cell exhaustion and restore anti-tumor immunity. J Exp Med 207, 2187-2194.
48. Samelson, L. E. (2002). Signal transduction mediated by the T cell antigen receptor: the role of adapter proteins. Annu Rev Immunol 20, 371-394.
49. Sarris, M., Andersen, K. G., Randow, F., Mayr, L., and Betz, A. G. (2008). Neuropilin-1 expression on regulatory T cells enhances their interactions with dendritic cells during antigen recognition. Immunity 28, 402-413.
50. Sasaki, Y., Ihara, K., Matsuura, N., Kohno, H., Nagafuchi, S., Kuromaru, R., Kusuhara, K., Takeya, R., Hoey, T., Sumimoto, H., and Hara, T. (2004). Identification of a novel type 1 diabetes susceptibility gene, T-bet. Hum Genet 115, 177-184.
51. Sharpe, A. H., and Freeman, G. J. (2002). The B7-CD28 superfamily. Nature Rev. Immunol. 2 116-126.
52. Sharpe, A. H., Wherry, E. J., Ahmed, R., and Freeman, G. J. (2007). The function of programmed cell death 1 and its ligands in regulating autoimmunity and infection. Nat Immunol 8, 239-245.
53. Steinmetz, O. M., Turner, J. E., Paust, H. J., Lindner, M., Peters, A., Heiss, K., Velden, J., Hopfer, H., Fehr, S., Krieger, T., et al. (2009). CXCR3 mediates renal Th1 and Th17 immune response in murine lupus nephritis. J Immunol 183, 4693-4704.
54. Sutherland, A. P., Joller, N., Michaud, M., Liu, S. M., Kuchroo, V. K., and Grusby, M. J. (2013). IL-21 promotes CD8+ CTL activity via the transcription factor T-bet. J Immunol 190, 3977-3984.
55. Tantisira, K. G., Hwang, E. S., Raby, B. A., Silverman, E. S., Lake, S. L., Richter, B. G., Peng, S. L., Drazen, J. M., Glimcher, L. H., and Weiss, S. T. (2004). TBX21: a functional variant predicts improvement in asthma with the use of inhaled corticosteroids. Proc Natl Acad Sci USA 101, 18099-18104.
56. von Herrath, M. G., Berger, D. P., Homann, D., Tishon, T., Sette, A., and Oldstone, M. B. (2000). Vaccination to treat persistent viral infection.
57. Virology 268, 411-419.
58. Weiss, A., and Littman, D. R. (1994). Signal transduction by lymphocyte antigen receptors. Cell 76, 263-274.
59. West, E. E., Jin, H. T., Rasheed, A. U., Penaloza-Macmaster, P., Ha, S. J., Tan, W. G., Youngblood, B., Freeman, G. J., Smith, K. A., and Ahmed, R. (2013). PD-L1 blockade synergizes with IL-2 therapy in reinvigorating exhausted T cells. J Clin Invest 123, 2604-2615.
60. Wherry, E. J., and Ahmed, R. (2004.). Memory CD8 T-cell differentiation during viral infection. J. Virol. 78, 5535-5545.
61. Wherry, E. J., Blattman, J. N., Murali-Krishna, K., van der Most, R., and Ahmed, R. (2003.). Viral persistence alters CD8 T-cell immunodominance and tissue distribution and results in distinct stages of functional impairment. J. Virol. 77, 4911-4927.
62. Williams, M. A., and Bevan, M. J. (2007). Effector and memory CTL differentiation. Annu Rev Immunol 25, 171-192.
63. Woodgett, J. R. (1990). Molecular cloning and expression of glycogen synthase kinase-3/factor A. Embo J 9, 2431-2438.
64. Woodgett, J. R. (2001). Judging a protein by more than its name: GSK-3. Sci STKE 2001, re12.
65. Xu, D., Fu, H. H., Obar, J. J., Park, J. J., Tamada, K., Yagita, H., and Lefrancois, L. (2013). A potential new pathway for PD-L1 costimulation of the CD8-T cell response to Listeria monocytogenes infection. PloS one 8, e56539.
66. Xu, W., and Zhang, J. J. (2005). Stat1-dependent synergistic activation of T-bet for IgG2a production during early stage of B cell activation. J Immunol 175, 7419-7424.
67. Zhu, Q., Yang, J., Han, S., Liu, J., Holzbeierlein, J., Thrasher, J. B., and Li, B. (2011). Suppression of glycogen synthase kinase 3 activity reduces tumor growth of prostate cancer in vivo. Prostate 71, 835-845.

The contents of the all of references cited in this application are incorporated by reference in their entirety herein.

Having described the invention and exemplary embodiments thereof, the invention is further described by the claims which follow.

1-85. (canceled)

86. A method for promoting T cell immunity, comprising administering an effective amount of at least one GSK-3 inhibitor to a subject in need thereof.

87. The method of claim 86, wherein said at least one GSK-3 inhibitor is selected from the group consisting of compounds that inhibit one GSK-3α, compounds that inhibit GSK-3β, compounds that inhibit GSK-3β2, and any combination thereof.

88. The method of claim 86, wherein said at least one GSK-3 inhibitor is selected from the group consisting of chemical compounds, antibodies, antibody fragments, anti-sense RNAs, small hairpin loop RNAs (shRNA), small interfering RNAs (siRNA), and any combination thereof.

89. The method of claim 86, wherein said method further comprises administering at least one immune modulatory compound other than a GSK-3 inhibitor.

90. The method of claim 89, wherein said at least one immune modulatory compound other than a GSK-3 inhibitor is selected from the group consisting of chemical compounds; PD-1 antagonists; CTLA-4 antagonists; anti-PD-1 antibodies or fragments thereof; anti-CTLA-4 antibodies or fragments thereof; cytokines; IFNγ; IL-12; IL-18; IL-21; antagonists or agonists of a receptor or ligand expressed by an immune cell; antagonists or agonists of a B7/CD28 or TNF receptor or ligand; antibodies that bind to a B7/CD28 or TNF receptor or ligand; fusion proteins comprising a B7/CD28 or TNF receptor or ligand; agents that inhibit the activity of an NK inhibitory receptor or promote the activity of an NK activating receptor; agents that specifically bind to PD-1, PD-L1, PD-L2, CTLA-4, LAG3, Tim3, VISTA or another modulatory receptor expressed on the surface of T cells; antibodies that bind PD-1, PD-L1, PD-L2, CTLA-4, LAG3, Tim3, VISTA or another modulatory receptor expressed on the surface of T cells; antibodies that bind to CD28, CD40, 4-1BB, or CD27; antibodies which enhance Th1 and CTL responses and/or reduce the development of Th2 or Th17 cells; agents that increase the transcription of cytokine receptors; and any combination thereof.

91. The method of claim 90, wherein said at least one immune modulatory compound other than a GSK-3 inhibitor is selected from the group consisting of antagonists or agonists of a B7/CD28 or TNF receptor or ligand; antibodies that bind to a B7/CD28 or TNF receptor or ligand; fusion proteins comprising a B7/CD28 or TNF receptor or ligand; and any combination thereof, wherein said receptors and ligands are selected from the group consisting of B7.1 (CD80), B7.2 (CD86), B7-DC (PD-L2 or CD273), B7-H1, B7-H2, B7-H3 (CD276), B7-H4 (VTCN1), B7-H5 (VISTA), B7-H6 (NCR3LG1), B7-H7 (HHLA2), PD-1 (CD279), PD-L3, CD28, CTLA-4 (CD152), ICOS(CD278), BTLA, NCR3, CD28H, NKp30, CD40, CD40L (CD154), LTα, LTβ, LT-(3R, FASL (CD178), CD30, CD30L (CD153), CD27, CD27L (CD70), OX40, OX40L, TRAIL/APO-2L, 4-1BB, 4-1BBL, TNF, TNF-R, TNF-R2, TRANCE, TRANCE-R, glucocorticoid-induced TNF receptor (GITR), GITR ligand, RELT, TWEAK, FN14, TNFα, TNFβ, RANK, RANK ligand, LIGHT, HVEM, GITR, TROY, RELT, and any combination thereof.

92. The method of claim 86, wherein said subject has a condition selected from the group consisting of cancers, proliferative conditions other than cancer, infectious diseases, and any combination thereof.

93. The method of claim 92, wherein said subject has a cancer selected from the group consisting of carcinomas, lymphomas, blastomas, sarcomas, leukemias, and any combination thereof.

94. The method of claim 92, wherein said subject has a cancer selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Müllerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sézary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Warthin's tumor, Wilms' tumor, B-cell lymphoma, low grade/follicular non-Hodgkin's lymphoma (NHL), small lymphocytic (SL) NHL, intermediate grade/follicular NEIL, intermediate grade diffuse NHL, high grade immunoblastic NEIL, high grade lymphoblastic NEIL, high grade small non-cleaved cell NEIL, bulky disease NHL, mantle cell lymphoma, AIDS-related lymphoma, Waldenström's Macroglobulinemia, chronic lymphocytic leukemia (CLL), acute lymphoblastic leukemia (ALL), Hairy cell leukemia, chronic myeloblastic leukemia, multiple myeloma and post-transplant lymphoproliferative disorder (PTLD), melanoma, ovarian cancer, brain cancer, solid tumors, stomach cancer, oral cancer, testicular cancer, uterine cancer, scleroderma, bladder cancer, esophageal cancer, and any combination thereof.

95. The method of claim 92, wherein said subject has an infectious disease selected from the group consisting of infectious diseases caused by bacteria, viruses, yeast or other fungi, parasites, and any combination thereof.

96. The method of claim 86, wherein said administration (i) downregulates PD-1 transcription or expression in immune cells of said subject, and/or (ii) upregulates Tbet transcription or expression in immune cells of said subject.

97. The method of claim 86, wherein said method further comprises (i) monitoring PD-1 transcription or expression in immune cells of said subject, and/or (ii) monitoring Tbet transcription or expression in immune cells of said subject, before, during and/or after treatment.

98. The method of claim 97, wherein said immune cells are selected from the group consisting of T cells, B cells, dendritic cells, macrophages, monocytes, myeloid cells, natural killer cells, mast cells, and any combination thereof.

99. The method of claim 97, wherein said immune cells are T cells selected from the group consisting of TH1 cells, CD4⁺ cells, CD8⁺ cells, and any combination thereof.

100. The method of claim 86, wherein said subject prior to treatment has (i) an increased incidence or number of immune cells including T cells that express PD-1; (ii) immune cells including T cells characterized by higher than normal levels of PD-1 expression; (iii) a decreased incidence or number of immune cells including T cells that express Tbet; and/or (iv) immune cells including T cells characterized by lower than normal levels of Tbet expression.

101. A method for reducing T cell immunity, comprising administering an effective amount of at least one compound that promotes the expression and/or activation of at least one isoform of GSK-3 to a subject in need thereof.

102. The method of claim 101, wherein said method further comprises (i) monitoring PD-1 transcription or expression in immune cells of said subject, and/or (ii) monitoring Tbet transcription or expression in immune cells of said subject.

103. The method of claim 101, wherein said method further comprises administering at least one immune modulatory compound other than a GSK-3 inhibitor.

104. The method of claim 101, wherein said subject has a condition selected from the group consisting of autoimmune, allergic and inflammatory conditions.

105. The method of claim 101, wherein said at least one compound that promotes the expression and/or activation of at least one GSK-3 isoform is selected from the group consisting of Pyk2, Fyn, Src, Csk, octreotide, lysophosphatidic acid, leucine-rich repeat kinase 2 (LRRK2), 6-hydroxydopamine, sphingolipids, psychosine, and any combination thereof.


SEQUENCE LISTING (6 sequences)

SEQ ID NO: 1 - DNA, 21 bp, Artificial Sequence
  PD-1-FW
ccgccttctgtaatggtttga

SEQ ID NO: 2 - DNA, 21 bp, Artificial Sequence
  PD-1-RV
gggcagctgtatgatctggaa

SEQ ID NO: 3 - DNA, 21 bp, Artificial Sequence
  GAPDH-FW
caacagcaactcccactcttc

SEQ ID NO: 4 - DNA, 22 bp, Artificial Sequence
  GAPDH-RW
ggtccagggtttcttactcctt

SEQ ID NO: 5 - DNA, 20 bp, Artificial Sequence
  TBET-FW
gatcgtcctgcagtctctcc

SEQ ID NO: 6 - DNA, 20 bp, Artificial Sequence
  TBET-RW
aactgtgttcccgaggtgtc